(19) United States
(12) Patent Application Publication
(10) Pub. No.: US 2020/0234789 A1
(43) Pub. Date: Jul. 23, 2020

(54) DESIGNED PROTEINS FOR LIGAND BINDING

(71) Applicants: THE REGENTS OF THE UNIVERSITY OF CALIFORNIA, Oakland, CA (US); DUKE UNIVERSITY, Durham, NC (US)

(72) Inventors: William DeGrado, San Francisco, CA (US); Nicholas Polizzi, Oakland, CA (US); Michael Therien, Durham, NC (US)

(21) Appl. No.: 16/633,809

(22) PCT Filed: Jul. 27, 2018
(86) PCT No.: PCT/US2018/044195

§ 371 Date: Jan. 24, 2020

Related U.S. Application Data
(60) Provisional application No. 62/537,774, filed on Jul. 27, 2017.

(51) Int. Cl. G16B 15/30; G06F 30/27

(52) U.S. Cl. CPC G16B 15/30; G06F 30/27

(57) ABSTRACT

Disclosed herein, inter alia, are methods and systems for optimizing protein ligand interactions for highly accurate de novo protein design.

### CROSS-REFERENCES TO RELATED APPLICATIONS

This application claims the benefit of U.S. Provisional Application No. 62/537,774, filed on Jul. 27, 2017, which is incorporated herein by reference in its entirety and for all purposes.

### STATEMENT AS TO RIGHTS TO INVENTIONS MADE UNDER FEDERALLY SPONSORED RESEARCH AND DEVELOPMENT

This invention was made with Government support under grant numbers GM-54616 and GM-071628 awarded by The National Institutes of Health, and grant numbers CHE-1413333, CHE-1413295 and DMR-1120901 awarded by The National Science Foundation. The Government has certain rights in the invention.

### REFERENCE TO A “SEQUENCE LISTING,” A TABLE, OR A COMPUTER PROGRAM LISTING APPENDIX SUBMITTED AS AN ASCII FILE

The Sequence Listing written in file 048536-593001WO Sequence Listing_ST25.txt, created Jul. 12, 2018, 7,148 bytes, machine format IBM-PC, MS Windows operating system, is hereby incorporated by reference.

### BACKGROUND

Many natural proteins contain precisely oriented cofactors that enable their functions, yet the de novo design of proteins that bind cofactors with atomic-scale precision has remained a significant challenge. De novo protein design critically tests our understanding of protein folding and function, and can provide new frameworks that combine man-made materials with protein scaffolds. Highly accurate design of porphyrin-binding proteins, validated by high-resolution structure determination, has presented a major unsolved challenge. Disclosed herein, inter alia, are solutions to these and other problems in the art.

### BRIEF SUMMARY

In an aspect is provided a computer-implemented method, including: a) identifying a set of ligand binding amino acid residues within a protein for binding to a ligand, wherein each ligand binding amino acid residue within the protein is associated with a set of ligand binding amino acid residue atomic coordinates and each atom of the ligand is associated with a set of ligand atomic coordinates; b) identifying a set of core amino acid residues within the protein that do not bind to the ligand, each core amino acid residue within the protein is associated with a set of core amino acid residue atomic coordinates; and c) optimizing the set of ligand binding amino acid residues; the set of ligand binding amino acid residue atomic coordinates; the set of core amino acid residues; and the set of core amino acid residue atomic coordinates; wherein the optimization is performed using at least an energy minimization calculation, and wherein the optimization is performed to energetically stabilize the protein.

In an aspect is provided a system, including: at least one data processor; and at least one memory storing instructions which, when executed by the at least one data processor, result in operations including: a) identifying a set of ligand binding amino acid residues within a protein for binding to a ligand, wherein each ligand binding amino acid residue within the protein is associated with a set of ligand binding amino acid residue atomic coordinates and each atom of the ligand is associated with a set of ligand atomic coordinates; b) identifying a set of core amino acid residues within the protein that do not bind to the ligand, each core amino acid residue within the protein is associated with a set of core amino acid residue atomic coordinates; and c) optimizing the set of ligand binding amino acid residues; the set of ligand binding amino acid residue atomic coordinates; the set of core amino acid residues; and the set of core amino acid residue atomic coordinates; wherein the optimization is performed using at least an energy minimization calculation, and wherein the optimization is performed to energetically stabilize the protein.

In another aspect is provided a non-transitory computer-readable storage medium including program code, which when executed by at least one data processor, causes operations including: a) identifying a set of ligand binding amino acid residues within a protein for binding to a ligand, wherein each ligand binding amino acid residue within the protein is associated with a set of ligand binding amino acid residue atomic coordinates and each atom of the ligand is associated with a set of ligand atomic coordinates; b) identifying a set of core amino acid residues within the protein that do not bind to the ligand, each core amino acid residue within the protein is associated with a set of core amino acid residue atomic coordinates; and c) optimizing the set of ligand binding amino acid residues; the set of ligand binding amino acid residue atomic coordinates; the set of core amino acid residues; and the set of core amino acid residue atomic coordinates; wherein the optimization is performed using at least an energy minimization calculation, and wherein the optimization is performed to energetically stabilize the protein.

In an aspect is provided a protein sequence obtainable based on the energy minimization calculation using the method, the system, or the non-transitory computer-readable medium as described herein.

In an aspect is provided a protein, or conservatively modified variant thereof, having the sequence:

(SEQ ID NO: 1)
EFEKLRQTGDELVQAFQRLREIFDKGDDDSLEQVLEEIEELIQKHRQLEDN
RQEAADTEAAKQGDQWVQLFQRFREAIDKGDKDSLEQLLEELEQALQKIRE
LAEKKN.

### BRIEF DESCRIPTION OF THE DRAWINGS

FIGS. 1A-1C. The design strategy. FIG. 1A: Structures of natural cofactor-binding proteins show a folded core supporting a cofactor-binding region. FIG. 1B: Examples of previously designed tetra-helical porphyrin-binding proteins; all but PS1 (which is described herein) lack a folded core. The α2 protein is from ref 40; the remainder are described in the text.

FIG. 1C: The design process starts with a parameterized backbone, which undergoes simultaneous optimization of packing of core residues (shown as spheres) in the binding region (light color) and folded core (dark color), with flexible backbone. The resultant holo-protein is tightly packed both in the binding region and in the folded core, whereas the apo-protein is tightly packed only in the folded core, which anchors the under-packed binding region to bind the cofactor. cytochrome b562 (pdb 256b), DHFR, dihydrofolate reductase (pdb 8dfr), flavodoxin (pdb 1czu).

FIG. 2. The computational design workflow for optimized core packing. The abiological porphyrin cofactor, (CF₃)₄PZn, is shown in the upper left. The constrained, parameterized backbone of SCRPZ-2 feeds into a flexible backbone design protocol that allows the interior side chains and backbone to simultaneously conform to the porphyrin (CF₃)₄PZn. On the right are depicted the ab initio folding predictions of PS1 sequence. The Rosetta folding algorithm predicts a shallow folding funnel for the binding region (light gray) and a deep folding funnel shifted toward lower RMSD for the folded core (dark gray) of apo-PS1. The RMSD (root mean squared deviation) in A is against the helical residues within these regions in the designed model. Energy is in Rosetta energy units (r.e.u.).

FIGS. 3A-3D. Biophysical characterization of apo- and holo-PS1. FIG. 3A: Electronic absorption and emission spectra of (CF₃)₄PZn/PS1 holo-protein and (CF₃)₄PZn in toluene solvent. Inset shows normalized emission spectrum of (CF₃)₄PZn upon electronic excitation at 405 nm (OD=0.1 at excitation wavelength); buffer=100 mM NaCl, 50 mM NaPi, pH 7.5. FIG. 3B: Determination of K_{D} by apo-PS1 titration into a buffer solution (100 mM NaCl, 50 mM NaPi, pH 7.5) of (CF₃)₄PZn with 1% w/v octyl-b-D-glucopyranoside. Inset shows spectral shifts upon porphyrin binding to PS1. FIG. 3C: Circular dichroism (CD) spectra of apo- and holo-PS1 in 50 mM NaPi, 100 mM NaCl, pH 7.5 as a function of temperature. The transitions appear reversible based on the fact that the spectra are identical after cooling to room temperature. Units are in molar residue ellipticity. Electronic absorbance spectra indicate holo-PS1 retains the porphyrin upon cooling. FIG. 3D: Pump-probe transient absorption spectra of (CF₃)₄PZn bound in the interior of holo-PS1 at 21° C. and 100° C. The black spectrum shows characteristic S₁→S_{N} absorptions of (CF₃)₄PZn, which smoothly transitions into the gray spectrum showing characteristic T₁→T_{N} absorptions of (CF₃)₄PZn. Inset exemplifies identical transient dynamics (primarily intersystem crossing from S₁ to T₁) at ΔAbs.=482 nm (scaled). Experimental conditions: solvent=50 mM NaPi, 100 mM NaCl, pH 7.5; excitation wavelength=600±5 nm; magic-angle polarization between pump and probe pulses; pump-probe cross-correlation of ˜250 fs.

FIG. 4. The structure of holo-PS1 agrees closely with the design. The structure of holo-PS1 superimposed on the design, with mean helical backbone RMSD of 0.8±0.1 Å. The holo-PS1 model shown is the centroid of the NMR structural ensemble. 26 porphyrin-protein nuclear Overhauser effects (NOEs), drawn as sticks, experimentally determine the orientation of the porphyrin within the binding site of PS1. Middle panel compares observed vs. designed orientations. All hydrophobic and helical backbone heavy atoms within 4 Å of porphyrin heavy atoms in the design were used for alignment (0.9±0.1 Å all-atom RMSD). Panel shows ˜10 Å slices of the holo-PS1 NMR centroid and design in the binding region and folded core, respectively.

FIGS. 5A-5F. Apo- and holo-PS1 share similar folded cores and differ in the binding region. FIG. 5A: 2D ¹H-¹⁵N HSQC spectra acquired for apo- and holo-PS1. Experimental conditions: 0.78 mM at 298K, 50 mM NaPi, 100 mM NaCl, pH 7.5, in 5% D₂O. Resonance assignments are indicated using the one-letter amino acid code. Signals arising from side chains (Asn HD2/ND2, Gln HE2/NE2, Arg HE/NE and Trp HE1/NE1) are also labeled. The residues belonging to the binding region and folded core are color-coded as in (FIG. 5B). Non-helical residues are labeled in cyan font face. The inset in the HSQC spectrum of apo-PS1 shows the chemical shift of the indole proton of Trp68 near 10.2 ppm. A dashed box surrounds 90% of the backbone resonances of apo-PS1 and is also placed at the same position in the holo-PS1 spectrum. Arrows point to resonances of residues within the binding region that change dramatically upon binding of the cofactor. FIG. 5B: Solution NMR structures of apo-PS1 and holo-PS1. The structures were aligned to the backbone of the helical folded core of the lowest energy holo-PS1 model. Terminal residues 1, 108, and 109 are not shown for clarity. FIG. 5C: Hydrogen-deuterium exchange protection factors (PF) measured for apo- and holo-PS1, mapped onto the centroid structure of holo-PS1. Backbone amide nitrogens of residues with determined PFs are shown as spheres. Not shown: N of Trp68 indole side chain is protected in holo, but not apo. FIGS. 5D-5F: Backbone alignment of the holo- and apo-centroids at the folded core shows, FIG. 5F, agreement of side chain rotamer states far from the binding site and, e, differences in first-shell rotamers (e.g., Trp68, Leu98) accompanied by changes in backbone of the binding region. Centroids are from NMR structural ensembles clustered via RMSD of core side chain heavy atoms.

FIG. 6. PS1 design metrics. PS1 design ensemble resulting from flexible backbone sequence design. FIG. 6B: Residues (Ca atoms shown as spheres) within the PS1 design that were allowed to vary from the SCRPZ-2 sequence. 40 of the 108 residues were allowed to vary, and, of the 40 residues, 28 were mutated and 12 residues were retained from the original SCRPZ-2 sequence as a result of the computational design process.

FIG. 7A-7B. Analytical ultracentrifugation and gel filtration analysis show that apo- and holo-PS1 are monomeric in solution. FIG. 7A: Analytical ultracentrifugation. Solutions of apo- and holo-PS1 were centrifuged at speeds ranging from 25,000 r.p.m. to 45,000 r.p.m. and monitored by absorbance at 280 nm. Parameters were globally fit to the data. Single-species fitting agrees well with the data over the entire range and yields the molecular weight of apo-PS1 15.81±0.09 kD and holo-PS1 12.24±0.91 kD, which agrees well with the 12.86 kD weight of PS1. At high concentration, the fit for apo-PS1 is not ideal, suggesting a small degree of aggregation. Partial specific volumes were estimated from SEDNTERP¹⁵ for amino acid side chains. FIG. 7B: Analytical gel filtration analysis of apo- and holo-PS1. Detection wavelengths are labeled as the same color as their respective curves. Apo shows a small degree (<5%) of dimerization (1.35 ml elution volume) relative to the monomer peak (1.62 ml elution volume). The small peak near 1.05 ml elution volume in holo-PS1 is unbound (excess), aggregated porphyrin eluting in the void volume of the superdex 75 5/150 column. Samples were run at concentrations of 100 μM and 37 μM for apo and holo, respectively, in 50 mM NaPi, 150 mM NaCl, pH 7.0 buffer.

FIG. 8. Temperature and GnHCl induced unfolding of apo-PS1. CD spectra at 222 nm of apo-PS1 as a function of temperature and denaturant (Guanidine HCl, GnHCl) concentration in 50 mM NaPi, 100 mM NaCl, pH 7.5 buffer. The midpoint for GnHCl-induced unfolding at 95° C. was approximately 4.5 M.

FIG. 9. Scaled absorption spectra of (CF₃)₄PM/PS1 complexes, M=Zn²⁺, Fe²⁺. Loading of (CF₃)₄PFe into PS1 was ˜40-50%, likely due to the extreme insolubility of the porphyrin. The featured bands at 550-650 nm indicate that (CF₃)₄PFe is in a homogenous environment in the ferrous state. The broad absorbance centered at 350 nm is also observed for (CF₃)₄PFe dissolved in organic solvent¹⁶, and does not reflect aggregation in water. The peak at 423 nm is also indicative of a homogenous binding environment. The absorption spectra were scaled to reflect the relative extinction coefficients of the porphyrins. Buffer=50 mM NaPi, 100 mM NaCl, pH 7.5.

FIG. 10. Absorption spectra of (CF₃)₄PZn/PS1 and (CF₃)₄PZn/PS2 complexes. Each protein shows 100% porphyrin loading, based on absorbance at 280 nm and 423 nm. Experimental conditions: buffer=100 mM NaCl, 50 mM NaPi, pH 7.5.

FIG. 11. The NMR structural ensemble of apo-PS1 contains two clusters of conformations, closed and open. Above, color mapping of the pairwise backbone RMSD matrix of each NMR ensemble member of apo-PS1. Apo models with high structural similarity in the region of residues 61-67 and 99-105 (labeled in the open structure shown below) are blue in the plot. Models that are structurally dissimilar (large RMSD) are red in the plot. Below, the model centroids representing the closed and open structures (models 1 and 18, respectively, in the deposited NMR structure). The porphyrin (CF₃)₄PZn is shown in green, and the holo centroid (orange) is also drawn for comparison.

FIG. 12. HDX protection factors for apo- and holo-PS1, as described in Table S5. Note that “68 indole” denotes the indole N of Trp68 side chain.

FIG. 13. Molecular dynamics simulations show the binding region of apo-PS1 is more accessible to solvent. Histogram of number of waters within 3.5 Å of any heavy atom of each buried amino acid side chain (an A or D position of the heptad repeat), from 1000 snapshots of a 1 μs trajectory of apo-PS1. All histograms are drawn to the same scale and show number of solvating waters normalized by side chain surface area. Binding region shown in light gray, and folded core in dark gray.

FIG. 14 depicts a flowchart illustrating a process for designing proteins, in accordance with some example embodiments.

FIG. 15 depicts a block diagram illustrating a computing system, in accordance with some example embodiments.

FIG. 16. Solution NMR structure of PS1 and computational models of PS1 variants.

FIG. 17. The PS1 deletion variant binds endogenous heme when expressed in E. coli. Characteristic Soret and Q bands of heme can be seen at 410 and 550 nm in the displayed absorption spectra.

FIGS. 18A-18B. enFold proteins are capable of noncovalently binding endogenous ligands in the cell. FIG. 18A: Expression in E. coli of the deletion variant of PS1 (PS1 D103-109, SEQ ID NO:8) shows a high loading of endogenous heme in the porphyrin binding site. Inset: E. coli cultures of after induction). FIG. 18B: Denovo proteins from binary sequence patterning. Previous studies have only been able to incorporate heme exogenously, i.e. after expression and purification, heme is added to the purified apo-protein. See Patel et al, Protein Science, 18:1388-1400.

### DETAILED DESCRIPTION

Protein catalysis requires atomic-level orchestration of side chains, substrates, and cofactors, yet the ability to design a small-molecule-binding protein entirely from first principles with a precisely predetermined structure has not been demonstrated. Herein we describe a novel protein, PS1, which binds a highly electron-deficient, non-natural porphyrin at temperatures up to 100° C. The high-resolution structure of holo-PS1 is in sub-A agreement with the design. The structure of apo-PS1 retains the remote core packing of the holo, predisposing a flexible binding region for the desired ligand-binding geometry. Our results illustrate the unification of core packing and binding site definition as a central principle of ligand-binding protein design.

### I. DEFINITIONS

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

A “detectable agent” or “detectable moiety” is a composition detectable by appropriate means such as spectroscopic, photochemical, biochemical, immunochemical, chemical, magnetic resonance imaging, or other physical means. For example, useful detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y. ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g. fluorescent dyes or chromophores), phosphor (e.g., phosphorescent dyes or chromophores), lumophore (luminescent dyes or chromophores), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g. carbon-11, nitrogen-13, oxygen-15, fluorine-18, rubidium-82), fluorodeoxyglucose (e.g. fluorine-18 labeled), any gamma ray emitting radionuclides, positron-emitting radionuclide, radiolabeled glucose, radiolabeled water, radiolabeled ammonia, biocolloids, microbubbles (e.g. including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g. iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, two-photon fluorophores, hyperpolarizable chromophores, or haptens and proteins or other entities which can be made detectable, e.g., by incorporating a radiolabel into a peptide or antibody specifically reactive with a target peptide. A detectable moiety is a monovalent detectable agent or a detectable agent capable of forming a bond with another composition.

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Ab, ²¹¹At, ²¹¹Pb, ²¹²Bi, ²¹²Pb, ²²³Ra, and ²²⁵Ac. Paramagnetic ions that may be used as additional imaging agents in accordance with the embodiments of the disclosure include, but are not limited to, ions of transition and lanthanide metals (e.g. metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

An amino acid residue in a protein “corresponds” to a given residue when it occupies the same essential structural position within the protein as the given residue.

The term “isolated” when applied to a nucleic acid or protein denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid. The terms “non-naturally occurring amino acid” and “unnatural amino acid” refer to amino acid analogs, synthetic amino acids, and amino acid mimetics, which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety. In embodiments, the protein includes at least 30 amino acid residues. A protein may be characterized as having a protein backbone. A “protein backbone” is used herein in accordance with its ordinary meaning and refers to the polymer of amino acid residues that create a continuous chain. For example, a protein backbone may refer to the series of amino acid residues covalently linked together, e.g.,

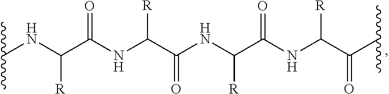

wherein each R independently represents optionally different amino acid side chains. In embodiments, the protein backbone includes core amino acid residues and ligand binding amino acid residues. In embodiments, the protein backbone includes core amino acid residues. In embodiments, the protein backbone includes ligand binding amino acid residues.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical”. This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

The term “amino acid side chain” refers to the functional substituent contained on amino acids. For example, an amino acid side chain may be the side chain of a naturally occurring amino acid. Naturally occurring amino acids are those encoded by the genetic code (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine), as well as those amino acids that are later modified, e.g., hydroxyproline, γ-carboxyglutamate, and O-phosphoserine. In embodiments, the amino acid side chain may be a non-natural amino acid side chain. In embodiments, the amino acid side chain is

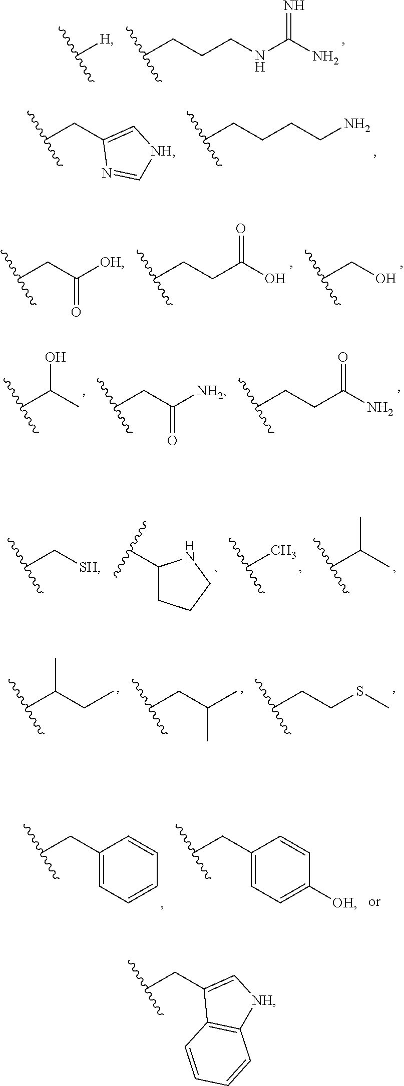

wherein the symbol “ [symbol] ” corresponds to the attachment of a chemical moiety (e.g., side chain) to the remainder of a molecule or chemical formula (e.g., the amino acid core, or

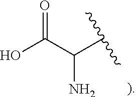 [SMILES: *C(N)C(=O)O]

The term “non-natural amino acid side chain” refers to the functional substituent of compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium, allylalanine, 2-aminoisobutryric acid. Non-natural amino acids are non-proteinogenic amino acids that either occur naturally or are chemically synthesized. Such analogs have modified R groups (e.g., norleucine) or modified peptide backbones, but retain the same basic chemical structure as a naturally occurring amino acid. Non-limiting examples include exo-cis-3-aminobicyclo[2.2.1]hept-5-ene-2-carboxylic acid hydrochloride, cis-2-aminocycloheptanecarboxylic acid hydrochloride,cis-6-amino-3-cyclohexene-1-carboxylic acid hydrochloride, cis-2-amino-2-methylcyclohexanecarboxylic acid hydrochloride, cis-2-amino-2-methylcyclopentanecarboxylic acid hydrochloride,2-(Boc-aminomethyl)benzoic acid, 2-(Boc-amino)octanedioic acid, Boc-4,5-dehydro-Leu-OH (dicyclohexylammonium), Boc-4-(Fmoc-amino)-L-phenylalanine, Boc-β-Homopyr-OH, Boc-(2-indanyl)-Gly-OH, 4-Boc-3-morpholineacetic acid, 4-Boc-3-morpholineacetic acid, Boc-pentafluoro-D-phenylalanine, Boc-pentafluoro-L-phenylalanine, Boc-Phe(2-Br)—OH, Boc-Phe(4-Br)—OH, Boc-D-Phe(4-Br)—OH, Boc-D-Phe(3-Cl)—OH, Boc-Phe(4-NH2)-OH, Boc-Phe(3-NO2)-OH, Boc-Phe(3,5-F2)-OH, 2-(4-Boc-piperazino)-2-(3,4-dimethoxyphenyl)acetic acid purum, 2-(4-Boc-piperazino)-2-(2-fluorophenyl)acetic acid purum, 2-(4-Boc-piperazino)-2-(3-fluorophenyl)acetic acid purum, 2-(4-Boc-piperazino)-2-(4-fluorophenyl)acetic acid purum, 2-(4-Boc-piperazino)-2-(4-methoxyphenyl)acetic acid purum, 2-(4-Boc-piperazino)-2-phenylacetic acid purum, 2-(4-Boc-piperazino)-2-(3-pyridyl)acetic acid purum, 2-(4-Boc-piperazino)-2-[4-(trifluoromethyl)phenyl]acetic acid purum, Boc-β-(2-quinolyl)-Ala-OH, N-Boc-1,2,3,6-tetrahydro-2-pyridinecarboxylic acid, Boc-β-(4-thiazolyl)-Ala-OH, Boc-β-(2-thienyl)-D-Ala-OH, Fmoc-N-(4-Boc-aminobutyl)-Gly-OH, Fmoc-N-(2-Boc-aminoethyl)-Gly-OH, Fmoc-N-(2,4-dimethoxybenzyl)-Gly-OH, Fmoc-(2-indanyl)-Gly-OH, Fmoc-pentafluoro-L-phenylalanine, Fmoc-Pen(Trt)-OH, Fmoc-Phe(2-Br)—OH, Fmoc-Phe(4-Br)—OH, Fmoc-Phe(3,5-F₂)—OH, Fmoc-β-(4-thiazolyl)-Ala-OH, Fmoc-β-(2-thienyl)-Ala-OH, 4-(hydroxymethyl)-D-phenylalanine.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about means the specified value.

The terms “bind” and “bound” as used herein is used in accordance with its plain and ordinary meaning and refers to the association between atoms or molecules. The association can be direct or indirect. For example, bound atoms or molecules may be direct, e.g., by covalent bond or linker (e.g. a first linker or second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi or hyrdophobic effects), hydrophobic interactions and the like).

The terms “set of ligand binding amino acid residues” as used herein refers to at least two ligand binding amino acid residues. “Ligand binding amino acid residues” refer to amino acid residues which are capable of binding (e.g., has a measurable dissociation constant of binding, has a dissociation constant of binding less than 1 μM) to a ligand. In embodiments, the ligand binding amino acid residues refer to amino acid residues which bind to a ligand. Each ligand binding amino acid residue is associated with a set of ligand binding amino acid residue atomic coordinates (e.g., Cartesian coordinates, internal coordinates, polar coordinates, or spherical coordinates) which defines the ligand binding amino acid residue in space (e.g., Euclidean space). In embodiments, ligand binding amino acid residues refer to amino acid residues within about 1, 2, 3, 4, 5, 6, 7, 8, 9, 10, 15, 20, 25, or 30 Å from the ligand. In embodiments, ligand binding amino acid residues refer to amino acid residues within about 5 Å from the ligand. In determining the set of ligand binding amino acid residues, such factors such as the proximity of the amino acid to the ligand or the interactions between the amino acid and the ligand may influence the designation to be a “ligand binding amino acid residue.”

The term “dissociation constant” is used in accordance with its plain ordinary meaning and refers to the ligand concentration at which half of the proteins are occupied (i.e. bound to a ligand) at equilibrium. Typically, the dissociation constant has molar units (M). The smaller the dissociation constant, the more tightly bound the ligand is, or the higher the affinity between ligand and protein. For example, a ligand with a nanomolar (nM) dissociation constant binds more tightly to a particular protein than a ligand with a micromolar (μM) dissociation constant.

The terms “ligand” and “cofactor” are synonymous, and used in accordance with their plain ordinary meaning in chemistry and biochemistry and refer to an agent (e.g., compound, metal, ion, biomolecule, agonist, antagonist) which is capable of binding to a protein (e.g., a protein described herein). In embodiments, a ligand refers to an agent (e.g., compound, metal, ion, biomolecule) which is binds (e.g., covalently or non-covalently) to a protein. Typically, upon binding the ligand has an effect on the protein (e.g., structural change of the protein, modulation of signaling pathways). A ligand is associated with a set of ligand atomic coordinates (e.g., Cartesian coordinates, internal coordinates, polar coordinates, spherical coordinates) which define the ligand in space (e.g., Euclidean space). The ligand may be endogenous or exogenous. Non-limiting examples of ligands include a catalyst, detectable agent, therapeutic agent, biological agent, cytotoxic agent, magnetic resonance imaging (MRI) agent, positron emission tomography (PET) agent, radiological imaging agent, diagnostic agent, theranostic (e.g., a combined therapeutic and diagnostic agent), photodynamic therapy (PDT) agent, porphyrin, porphycene, rubyrin, rosarin, hexaphyrin, sapphyrin, chlorophyll, chlorin, phthalocyanine, porphyrazine, corrole, N-confused porphyrin, bacteriochlorophyll, pheophytin, texaphyrin, or related macrocyclic-based component that is capable of binding a metal ion. In embodiments, the ligand is a peptide (e.g., 2 to 30 amino acid residues), a protein (e.g., greater than 30 amino acid residues), a small molecule (e.g., a compound with a molecular weight of less than 2000 Daltons), or a small molecule-metal-ion complex (e.g., a metalloporphyrin). In embodiments, the ligand is endogenous. In embodiments, the ligand is exogenous. In embodiments, the ligand is flavin. In embodiments, the ligand is heme.

The terms “set of core amino acid residues” as used herein refers to at least two core amino acid residues. Core amino acid residues refer to amino acid residues, which are incapable of binding to a ligand (e.g., does not have a measurable dissociation constant of binding, does not have a dissociation constant of binding less than 1 μM). In embodiments core amino acids are amino acids which do not bind a ligand. Each core amino acid residue is associated with a set of core amino acid residue atomic coordinates (e.g., Cartesian coordinates, internal coordinates, polar coordinates, spherical coordinates) which defines the core binding amino acid residue in space (e.g., Euclidean space). Core amino acids are at least 75% inaccessible to a 1.8 Å spherical probe. A typical set of core amino acid residues contains at least 6 amino acid residues. In embodiments, the set of core amino acid residues includes amino acid residues which are solvent inaccessible as measured by the accessible surface area. Additional information regarding the accessible surface area assessment may be found in Lins et al. (Lins, L., Thomas, A., & Brasseur, R. (2003) Protein Science: A Publication of the Protein Society, 12(7), 1406-141), which is incorporated herein in its entirety for all purposes. In embodiments, the core amino acids atomic coordinates are greater than 5 Å from any ligand atomic acid coordinate. In embodiments, the set of core amino acid residues is hydrophobic. In embodiments, the core amino acids includes the sequence:

(SEQ ID NO: 5)
LGLVAFLIFGLVLILIHLFAAGWVFFAILLLLALILA.

The terms “optimizing” and “optimization” are used in accordance with their ordinary meaning in mathematics and computer science and refers to identifying a favorable outcome subject to certain criteria (e.g., constraints) from a set of available possibilities. Optimizing may employ iterative or heuristic algorithms, such as simplex algorithm, memetic algorithm, differential evolution algorithm, evolutionary algorithm, genetic algorithm, tabu algorithm, particle swarm algorithm, stimulated annealing algorithm, Monte Carlo sampling algorithm, dead-end elimination algorithm, branch and bound algorithm, or a pruning algorithm. For example, optimizing typically includes evaluating an energy function (e.g., force field model) and finding the minimum (e.g., global minimum or local minimum). Optimizing may include repeated evaluations of the energy function and may include fixing an atomic coordinate (e.g., fixing an atomic coordinate of at least one ligand binding amino acid residue atomic coordinate), introducing additional amino acid residues into the set of amino acid residues (e.g., the set of ligand binding amino acid residues), restricting the introduction of additional amino acid residues into the set of amino acid residues (e.g., the set of ligand binding amino acid residues), or a geometric transformation (e.g., translation or rotation) of an amino acid residue atomic coordinate (e.g., the atomic coordinate of the ligand binding amino acid residue atomic coordinates). The output of an optimization process may provide a set of ligand binding amino acid residues and a corresponding set of ligand binding amino acid residue atomic coordinates, and a set of core amino acid residues and a corresponding set of core amino acid residue atomic coordinates, which corresponds to an energetically stabilized protein. In embodiments the outcome of the optimization is the global minimum (e.g., the most energetically stabilized protein). In embodiments the outcome of the optimization is a local minimum (e.g., a minimum energy given the domain). In embodiments the optimization is complete when the derivative of the energy with respect to the position of the atoms, ∂E/∂r, is zero and the Hessian matrix has positive eigenvalues. In embodiments, optimizing includes a plurality of minimization calculations. In embodiments the optimization is a finite number of iterations.

An energy minimization calculation refers to the process of evaluating the energy as a function of the atomic coordinates, V(r). The energy function may include intra- and intermolecular energy terms within the system (e.g., protein) which may be written as Vₜₒₜₐₗ(r)=V_{bonds}(r)+V_{angles}(r)+V_{dihedral}(r)+Vᵢₘₚᵣₒₚₑᵣ(r)+V_{nonbonding}(r)+V_{electrostatics}(r); where Vₜₒₜₐₗ(r) corresponds to the total energy as a function of the atomic positions; V_{bonds}(r) corresponds to the energy contribution from bonded atoms, V_{angles}(r) corresponds to the energy contribution from angles; V_{dihedral}(r) corresponds to the energy contribution from dihedral torsions; Vᵢₘₚᵣₒₚₑᵣ(r) corresponds to the energy contribution from out-of-plane torsions; V_{nonbonding}(r) corresponds to the energy contribution from nonbonding interactions; and V_{electrostatics}(r) corresponds to the energy contribution from electrostatic interactions. Additional energy function terms may also be included in the total energy function, Vₜₒₜₐₗ(r), for example additional functions from molecular mechanics, functions from structural bioinformatics (log-odds scores), amino acid sidechain packing functions (e.g., functions and algorithms which vary the identity and rotamer of an amino acid side chain), protein radius of gyration functions, or a penalty function.

The term biomolecule as used herein refers to a molecule present in living organisms (e.g., proteins, carbohydrates, lipids, and nucleic acids, metabolites) and may be endogenous or exogenous in origin.

The term “energetically stabilized protein” is used in accordance with its ordinary meaning in the art, and is understood to refer to a protein which is structurally and thermodynamically stable relative to the protein that has not been energetically stabilized. For example, an energetically stabilized protein is determined to be energetically stabilized by determining the difference in the Gibbs free energy between the folded and unfolded states of the protein, also referred to herein as ΔG_{folding}. An energetically stabilized protein may be characterized by a well-dispersed NMR spectrum and/or the presence of a significantly folded core. In embodiments, the energetically stabilized protein is an enzyme. In embodiments, the energetically stabilized protein is an apo protein (e.g., a protein that is not bound to a ligand). In embodiments, the energetically stabilized protein is a holo protein (e.g., a protein that is bound to a ligand). In embodiments, the energetically stabilized protein is an apo protein which is capable of becoming a holo protein upon ligand binding. In embodiments, an energetically stabilized protein refers to a protein which is capable of performing a function (e.g., modulating a signal pathway). In embodiments, the energetically stabilized protein resists side-reactions such as aggregation and proteolysis. In embodiments, the energetically stabilized protein has a ΔG_{folding} of about −5 to about −40 kcal/mol in standard physiological conditions (e.g., temperature range of 20-40 degrees Celsius, atmospheric pressure of 1, pH of 6-8, glucose concentration of 1-20 mM, atmospheric oxygen concentration).

The term “exogenous” refers to a molecule or substance (e.g., a compound, ligand, or protein) that originates from outside a given cell or organism. Conversely, the term “endogenous” refers to a molecule or substance that is native to, or originates within, a given cell or organism.

A “therapeutic agent” as used herein refers to an agent (e.g., compound or composition) that when administered to a subject in sufficient amounts will have a therapeutic effect, such as an intended prophylactic effect, preventing or delaying the onset (or reoccurrence) of an injury, disease, pathology or condition, or reducing the likelihood of the onset (or reoccurrence) of an injury, disease, pathology, or condition, or their symptoms or the intended therapeutic effect, e.g., treatment or amelioration of an injury, disease, pathology or condition, or their symptoms including any objective or subjective parameter of treatment such as abatement; remission; diminishing of symptoms or making the injury, pathology or condition more tolerable to the patient; slowing in the rate of degeneration or decline; making the final point of degeneration less debilitating; or improving a patient's physical or mental well-being.

The term “small molecule” or the like as used herein refers, unless indicated otherwise, to a molecule having a molecular weight of less than about 700 Dalton, e.g., less than about 700, 650, 600, 550, 500, 450, 400, 350, 300, 250, 200, 100, or 50 Dalton.

In this disclosure, “comprises,” “comprising,” “containing” and “having” and the like can have the meaning ascribed to them in U.S. patent law and can mean “includes,” “including,” and the like. “Consisting essentially of or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

### II. METHODS

In an aspect is provided a computer-implemented method, including: a) identifying a set of ligand binding amino acid residues within a protein for binding to a ligand, wherein each ligand binding amino acid residue within the protein is associated with a set of ligand binding amino acid residue atomic coordinates and each atom of the ligand is associated with a set of ligand atomic coordinates; b) identifying a set of core amino acid residues within the protein that do not bind to the ligand, each core amino acid residue within the protein is associated with a set of core amino acid residue atomic coordinates; and c) optimizing the set of ligand binding amino acid residues; the set of ligand binding amino acid residue atomic coordinates; the set of core amino acid residues; and the set of core amino acid residue atomic coordinates; wherein the optimization is performed using at least an energy minimization calculation, and wherein the optimization is performed to energetically stabilize the protein. In embodiments, the optimization is performed to improve, relative to a control, the protein-ligand interactions (e.g., decrease the dissociation constant of binding 1-fold, 2-fold, 3-fold, 4-fold or 5-fold). In embodiments, the optimization modulates, relative to a control, the non-covalent interactions between the protein and the ligand.

In embodiments, step c) includes simultaneously optimizing the set of ligand binding amino acid residues; the set of ligand binding amino acid residue atomic coordinates; the set of core amino acid residues; and the set of core amino acid residue atomic coordinates. In embodiments, step c) includes concurrently (e.g., performing an optimization iteration on all sets prior to continuing the optimization) optimizing the set of ligand binding amino acid residues; the set of ligand binding amino acid residue atomic coordinates; the set of core amino acid residues; and the set of core amino acid residue atomic coordinates.

In embodiments, the optimizing is joint optimizing (e.g., optimizing the set of ligand binding amino acid residues, the set of core amino acid residues, and optionally the ligand simultaneously). In embodiments, step c) includes optimizing the set of ligand binding amino acid residues; the set of ligand binding amino acid residue atomic coordinates; the set of core amino acid residues; and the set of core amino acid residue atomic coordinates. In embodiments, step c) includes optimizing the set of ligand binding amino acid residues and the set of core amino acid residues. In embodiments, step c) includes optimizing the set of ligand binding amino acid residues and the set of ligand binding amino acid residue atomic coordinates. In embodiments, step c) includes optimizing the set of core amino acid residues and the set of core amino acid residue atomic coordinates. In embodiments, step c) includes optimizing the set of ligand binding amino acid residue atomic coordinates and the set of core amino acid residue atomic coordinates.

In embodiments, step c) includes optimizing the protein backbone. Optimizing the protein backbone may refer to repeated evaluations of the energy function and may include fixing an atomic coordinate (e.g., fixing an atomic coordinate of at least one ligand binding amino acid residue atomic coordinate, but not the side chain of the residue), introducing additional amino acid residues into the set of amino acid residues (e.g., the set of ligand binding amino acid residues), restricting the introduction of additional amino acid residues into the set of amino acid residues (e.g., the set of ligand binding amino acid residues), or a geometric transformation (e.g., translation or rotation) of an amino acid residue atomic coordinate, but not the side chain of the residue (e.g., the atomic coordinate of the ligand binding amino acid residue atomic coordinates). In embodiments, step c) includes simultaneously optimizing the protein backbone and the set of ligand binding amino acid residues. In embodiments, step c) includes simultaneously optimizing the protein backbone and the ligand. In embodiments, step c) includes simultaneously optimizing the protein backbone and the set of core amino acid residues. In embodiments, step c) includes optimizing the protein backbone using known conformational sampling techniques in the art (e.g., rigid-body shifts of helices, backrub algorithms, or crankshaft algorithms). In embodiments, step c) is performed using a protein modeling software suite (e.g., Rosetta). In embodiments, step c) includes an ensemble (e.g., a finite set of proteins, which includes amino acid residue atomic coordinates) of backbones for conformational sampling calculations.

In embodiments, step c) includes fixing (e.g., not geometrically displacing) an atomic coordinate of at least one ligand binding amino acid residue atomic coordinate; fixing an atomic coordinate of at least one ligand atomic coordinate; prohibiting introduction of an additional amino acid residue into the set of ligand binding amino acid residues; or prohibiting the deletion of an amino acid residue from the set of ligand binding amino acid residues.

In embodiments, step c) includes fixing an atomic coordinate of at least one ligand binding amino acid residue atomic coordinate. In embodiments, step c) includes fixing all atomic coordinates of at least one ligand binding amino acid residue atomic coordinate. In embodiments, step c) includes fixing an atomic coordinate of at least one ligand atomic coordinate. In embodiments, step c) includes fixing all atomic coordinates of the ligand atomic coordinate. In embodiments, step c) includes prohibiting introduction of an additional amino acid residue into the set of ligand binding amino acid residues. In embodiments, step c) includes prohibiting the deletion of an amino acid residue from the set of ligand binding amino acid residues. In embodiments, step c) includes prohibiting introduction of an additional amino acid residue into the set of core amino acid residues. In embodiments, step c) includes prohibiting the deletion of an amino acid residue from the set of core amino acid residues. In embodiments, the method includes distance and angle constraints (i.e. specifying the distance of a ligand to an amino acid (e.g., a ligand binding amino acid residue) coordinate).

In embodiments, the optimizing includes fixing (e.g., not geometrically displacing) at least one atomic coordinate of the ligand atomic coordinates. In embodiments, the optimizing does not include fixing at least one atomic coordinate of at least one core amino acid residue atomic coordinates. In embodiments, the optimizing does not include fixing at least one atomic coordinate of the core amino acid residue atomic coordinates. In embodiments, the optimizing does not include fixing any atomic coordinates of the core amino acid residue atomic coordinates. In embodiments, the optimizing includes fixing angle form by three atoms (e.g., angles formed between atoms of the ligand and the ligand bind amino acid residues) or fixing the distance between atoms (e.g., at least one atomic coordinate of the ligand and at least one atomic coordinate of the ligand binding amino acid residue).

In embodiments, the optimizing includes an iterative or heuristic algorithm. In embodiments, the optimizing includes an iterative algorithm. In embodiments, the optimizing includes a heuristic algorithm. In embodiments, the optimizing includes a simplex algorithm, memetic algorithm, differential evolution algorithm, evolutionary algorithm, genetic algorithm, tabu algorithm, particle swarm algorithm, or stimulated annealing algorithm. In embodiments, the optimizing includes a Monte Carlo sampling algorithm, dead-end elimination algorithm, branch and bound algorithm, or a pruning algorithm. In embodiments, the optimizing includes knobs-into-holes side chain packing. In embodiments, the optimization may begin with an idealized, parameterized backbone. In embodiments, optimization may relax the backbone structure of the protein, for example, by using gradient descent algorithms, while optimizing the protein sequence via rotamer sampling and minimization.

In embodiments, the optimizing includes introducing an additional ligand binding amino acid residue into the set of ligand binding amino acid residues, deleting a ligand binding amino acid residue from the set of ligand binding amino acid residues, a geometric transformation of at least one atomic coordinate of the ligand binding amino acid residue atomic coordinates.

In embodiments, the optimizing includes introducing an additional ligand binding amino acid residue into the set of ligand binding amino acid residues (e.g., designating an amino acid residue previously designated as a core amino acid residue to a ligand binding amino acid residue). In embodiments, the optimizing includes replacing a ligand binding amino acid residue within the set of ligand binding amino acid residues. In embodiments, the optimizing includes deleting a ligand binding amino acid residue from the set of ligand binding amino acid residues (e.g., designating an amino acid residue previously designated as a ligand amino acid residue to a core binding amino acid residue). In embodiments, the optimizing includes a geometric transformation of at least one atomic coordinate of the ligand binding amino acid residue atomic coordinates. In embodiments, the optimizing includes a geometric transformation of the atomic coordinates of at least one of the ligand binding amino acid residue atomic coordinates. In embodiments, the optimizing includes a geometric transformation of the atomic coordinates of the ligand binding amino acid residue atomic coordinates.

In embodiments, the geometric transformation includes a translation (i.e., a geometric transformation that moves a coordinate by the same distance in a given direction) or a rotation of at least one atomic coordinate of the ligand binding amino acid residue atomic coordinates. In embodiments, the geometric transformation includes a translation (e.g., displacing the x coordinate) of at least one atomic coordinate of the ligand binding amino acid residue atomic coordinates. In embodiments, the geometric transformation includes a translation of at least two atomic coordinates of the ligand binding amino acid residue atomic coordinates. In embodiments, the geometric transformation includes a translation of all atomic coordinates (e.g., x, y, and z coordinates in Cartesian space) of the ligand binding amino acid residue atomic coordinates. In embodiments, the geometric transformation includes a rotation of at least one atomic coordinate of the ligand binding amino acid residue atomic coordinates. In embodiments, the geometric transformation includes a rotation of at least two atomic coordinates of the ligand binding amino acid residue atomic coordinates. In embodiments, the geometric transformation includes a rotation of at least three atomic coordinates of the ligand binding amino acid residue atomic coordinates. In embodiments, the geometric transformation includes a rotation of all atomic coordinates of the ligand binding amino acid residue atomic coordinates.

In embodiments, the optimizing includes a geometric transformation of at least one atomic coordinate of the core amino acid residue atomic coordinates. In embodiments, the geometric transformation includes a translation or a rotation of at least one atomic coordinate of the core amino acid residue atomic coordinates. In embodiments, the geometric transformation includes a translation of at least one atomic coordinate of the core amino acid residue atomic coordinates. In embodiments, the geometric transformation includes a translation of at least two atomic coordinates of the core amino acid residue atomic coordinates. In embodiments, the geometric transformation includes a translation of all atomic coordinates of the core amino acid residue atomic coordinates. In embodiments, the geometric transformation includes a rotation of at least one atomic coordinate of the core amino acid residue atomic coordinates. In embodiments, the geometric transformation includes a rotation of at least two atomic coordinates of the core amino acid residue atomic coordinates. In embodiments, the geometric transformation includes a rotation of at least three atomic coordinates of the core amino acid residue atomic coordinates. In embodiments, the geometric transformation includes a rotation of all atomic coordinates of the core amino acid residue atomic coordinates.

In embodiments, the optimizing includes 1a) calculating the force on each atom in the protein (e.g., the set of ligand binding amino acid residues; the set of core amino acid residues; and the ligand); 2a) evaluating the calculation to determine if it is the minimum or below an acceptable threshold; 3a) if the force is less than a threshold, the optimization is finished, otherwise perform a geometric transformation (e.g., translation) of at least one atomic coordinate on the atoms in the protein; and 4a) repeat.

In embodiments, the geometric transformation of at least one atomic coordinate includes no greater than a 6 Å displacement of any atomic coordinate. In embodiments, the geometric transformation of at least one atomic coordinate includes no greater than a 3 Å displacement of any atomic coordinate. In embodiments, the displacement is no greater than 0.1, 0.2, 0.3, 0.4, 0.5, 1, 2, 3, 4, 5, 6, 7, 8, 9, or 10 Å displacement of any atomic coordinate. In embodiments, the displacement is no greater than 0.01, 0.02, 0.03, 0.04, 0.05, 0.1, 0.2, 0.3, 0.4, 0.5, 0.6, 0.7, 0.8, 0.9, or 1.0 Å displacement of any atomic coordinate.

In embodiments, the set of ligand binding amino acids includes at least 50 amino acid residues. In embodiments, the set of ligand binding amino acids includes at least 40 amino acid residues. In embodiments, the set of ligand binding amino acids includes at least 30 amino acid residues. In embodiments, the set of ligand binding amino acids includes at least 20 amino acid residues. In embodiments, the set of ligand binding amino acids includes at least 12 amino acid residues. In embodiments, the set of ligand binding amino acids includes at least 10 amino acid residues. In embodiments, the set of ligand binding amino acids includes at least 8 amino acid residues. In embodiments, the set of ligand binding amino acids includes at least 6 amino acid residues. In embodiments, the set of ligand binding amino acids includes at least 5 amino acid residues. In embodiments, the set of ligand binding amino acids includes at least 4 amino acid residues. In embodiments, the set of ligand binding amino acids includes at least 3 amino acid residues. In embodiments, the set of ligand binding amino acids includes at least 2 amino acid residues. In embodiments the ligand binding amino acids are apolar. In embodiments the ligand binding amino acids are hydrophilic.

In embodiments, the set of ligand binding amino acids includes 50 amino acid residues. In embodiments, the set of ligand binding amino acids includes 40 amino acid residues. In embodiments, the set of ligand binding amino acids includes 30 amino acid residues. In embodiments, the set of ligand binding amino acids includes 20 amino acid residues. In embodiments, the set of ligand binding amino acids includes 12 amino acid residues. In embodiments, the set of ligand binding amino acids includes 10 amino acid residues. In embodiments, the set of ligand binding amino acids includes 8 amino acid residues. In embodiments, the set of ligand binding amino acids includes 6 amino acid residues. In embodiments, the set of ligand binding amino acids includes 5 amino acid residues. In embodiments, the set of ligand binding amino acids includes 4 amino acid residues. In embodiments, the set of ligand binding amino acids includes 3 amino acid residues. In embodiments, the set of ligand binding amino acids includes 2 amino acid residues. In embodiments the ligand binding amino acids are polar. In embodiments the ligand binding amino acids are hydrophilic.

In embodiments, the energy minimization calculation includes a molecular mechanics function, a structural bioinformatics function, an amino acid sidechain packing function, a protein radius of gyration function, or a combination thereof. In embodiments, the energy minimization calculation includes a penalty function.

In embodiments, the core amino acids are at least 75% inaccessible to a 1.8 Å spherical probe. In embodiments, the core amino acids are at least 75% inaccessible to a 1.0 Å spherical probe. In embodiments, the core amino acids are at least 75% inaccessible to a 1.2 Å spherical probe. In embodiments, the core amino acids are at least 75% inaccessible to a 1.4 Å spherical probe. In embodiments, the core amino acids are at least 75% inaccessible to a 1.6 Å spherical probe. In embodiments, the core amino acids are at least 75% inaccessible to a 2.0 Å spherical probe. In embodiments, the core amino acids are at least 80% inaccessible to a 1.8 Å spherical probe. In embodiments, the core amino acids are at least 90% inaccessible to a 1.8 Å spherical probe. In embodiments, the core amino acids are at least 95% inaccessible to a 1.8 Å spherical probe. In embodiments, the set of core amino acids includes at least 50 amino acid residues. In embodiments, the set of core amino acids includes at least 40 amino acid residues. In embodiments, the set of core amino acids includes at least 30 amino acid residues. In embodiments, the set of core amino acids includes at least 20 amino acid residues. In embodiments, the set of core amino acids includes at least 12 amino acid residues. In embodiments, the set of core amino acids includes at least 10 amino acid residues. In embodiments, the set of core amino acids includes at least 8 amino acid residues. In embodiments, the set of core amino acids includes at least 6 amino acid residues. In embodiments the core amino acids are apolar. In embodiments the core amino acids are hydrophobic.

In embodiments, the set of core amino acids includes 6 amino acids. In embodiments, the set of core amino acids includes 8 amino acids. In embodiments, the set of core amino acids includes 10 amino acids. In embodiments, the set of core amino acids includes 20 amino acids. In embodiments, the set of core amino acids includes 30 amino acids. In embodiments, the set of core amino acids includes 40 amino acids. In embodiments, the set of core amino acids includes 35, 36, 37, 38, 39, or 40 amino acids. In embodiments, the set of core amino acids includes 37 amino acids. In embodiments, the core amino acids include the sequence: LGLVAFLIFGLVLILIHLFAAGWVFFAILLLLALILA (SEQ ID NO:5). In embodiments, the core amino acids include the sequence: LGIILLLAIGLILLAFHLFFAGWLFIAILLFSGIILA (SEQ ID NO:6).

In embodiments, the protein is 99% identical to SEQ ID NO:5. In embodiments, the protein is 98% identical to SEQ ID NO:5. In embodiments, the protein is 95% identical to SEQ ID NO:5. In embodiments, the protein is 90% identical to SEQ ID NO:5. In embodiments, the protein is 85% identical to SEQ ID NO:5. In embodiments, the protein is 80% identical to SEQ ID NO:5. In embodiments, the protein is 60% identical to SEQ ID NO:5. In embodiments, the protein is about 99% identical to SEQ ID NO:5. In embodiments, the protein is about 98% identical to SEQ ID NO:5. In embodiments, the protein is about 95% identical to SEQ ID NO:5. In embodiments, the protein is about 90% identical to SEQ ID NO:5. In embodiments, the protein is about 85% identical to SEQ ID NO:5. In embodiments, the protein is about 80% identical to SEQ ID NO:5. In embodiments, the protein is about 60% identical to SEQ ID NO:5.

In embodiments, the protein is 99% identical to SEQ ID NO:6. In embodiments, the protein is 98% identical to SEQ ID NO:6. In embodiments, the protein is 95% identical to SEQ ID NO:6. In embodiments, the protein is 90% identical to SEQ ID NO:6. In embodiments, the protein is 85% identical to SEQ ID NO:6. In embodiments, the protein is 80% identical to SEQ ID NO:6. In embodiments, the protein is 60% identical to SEQ ID NO:6. In embodiments, the protein is about 99% identical to SEQ ID NO:6. In embodiments, the protein is about 98% identical to SEQ ID NO:6. In embodiments, the protein is about 95% identical to SEQ ID NO:6. In embodiments, the protein is about 90% identical to SEQ ID NO:6. In embodiments, the protein is about 85% identical to SEQ ID NO:6. In embodiments, the protein is about 80% identical to SEQ ID NO:6. In embodiments, the protein is about 60% identical to SEQ ID NO:6.

In embodiments, the set of core amino acids includes at least 50% of the total number of amino acid residues in the protein.

In embodiments, the ligand is a porphyrin, porphycene, rubyrin, rosarin, hexaphyrin, sapphyrin, chlorophyll, chlorin, phthalocyanine, porphyrazine, corrole, N-confused porphyrin, bacteriochlorophyll, pheophytin, texaphyrin, or related macrocyclic-based component, that is capable of binding a metal ion. In embodiments, the ligand is a detectable agent. In embodiments, the ligand is a therapeutic agent, biological agent, cytotoxic agent, magnetic resonance imaging (MRI) agent, positron emission tomography (PET) agent, radiological imaging agent, diagnostic agent, theragostic, or a photodynamic therapy (PDT) agent. In embodiments, the ligand is a therapeutic agent. In embodiments, the ligand is a biological agent. In embodiments, the ligand is a cytotoxic agent (e.g., an anticancer agent). In embodiments, the ligand is a magnetic resonance imaging (MRI) agent. In embodiments, the ligand is a positron emission tomography (PET) agent. In embodiments, the ligand is a radiological imaging agent. In embodiments, the ligand is a diagnostic agent. In embodiments, the ligand is a theragostic agent. In embodiments, the ligand is a photodynamic therapy (PDT) agent. In embodiments, the ligand is a small molecule.

In embodiments, the ligand is a catalyst. In embodiments, the catalyst catalyzes an abiological or bio-orthogonal reaction. In embodiments, the ligand is a molecule that exists within a living system (e.g., within an organism or a cell). In embodiments, the ligand is (CF₃)-4PZn. In embodiments, the ligand is (CF₃)₄PFe. In embodiments, the ligand atomic coordinates are optimized using known methods in the art (e.g., density functional theory using the B3-LYP functional).

In embodiments, the method further includes synthesizing the protein (e.g., utilizing the expression vectors such as the plasmid method described in the Example, such as cloning into the IPTG-inducible pET-11a plasmid). In embodiments, the method further includes expressing the protein.

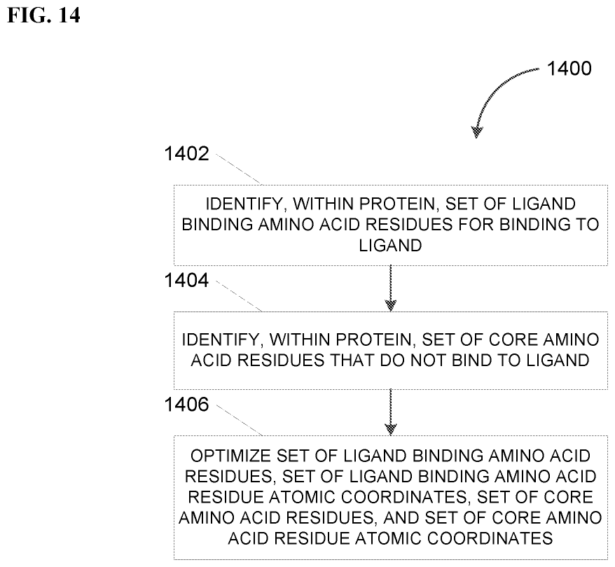

FIG. 14 depicts a flowchart illustrating a process 1400 for designing proteins, in accordance with some example embodiments. Referring to FIG. 14, the process 1400 can be performed in order to design an energetically stabilized protein (e.g., a protein that is structurally and thermodynamically stable as determined by the difference in the Gibbs free energy between the folded and unfolded states of the protein).

At 1402, a set of ligand binding amino acid residues within a protein for binding to a ligand can be identified. These ligand binding amino acid residues can form the backbone of a protein. Each ligand binding amino acid residue within the protein can be associated with a set of ligand binding amino acid residue atomic coordinates, which can define the ligand binding amino acid residue in space. Furthermore, each atom of the ligand can be associated with a set of ligand atomic coordinates, which can define the ligand in space. As noted herein, these coordinates can be Cartesian coordinates, internal coordinates, polar coordinates, spherical coordinates, and/or the like.

At 1404, a set of core amino acid residues within the protein that do not bind to the ligand can be identified. The backbone of the protein can further include core amino acid residues. Each core amino acid residue within the protein can be associated with a set of core amino acid residue atomic coordinates, which define the core amino acid residue in space.

At 1406, the set of ligand binding amino acid residues, the set of ligand binding amino acid residue atomic coordinates, the set of core amino acid residues, and the set of core amino acid residue atomic coordinates can be optimized. For example, the optimization can be performed using an energy minimization calculation including, for example, a molecular mechanics function, a structural bioinformatics function, an amino acid sidechain packing function, a protein radius of gyration function, and/or the like. Optimizing the set of ligand binding amino acid residues, the set of ligand binding amino acid residue atomic coordinates, the set of core amino acid residues, and the set of core amino acid residue atomic coordinates can generate an energetically stabilized protein.

### III. SYSTEMS AND MEDIUMS

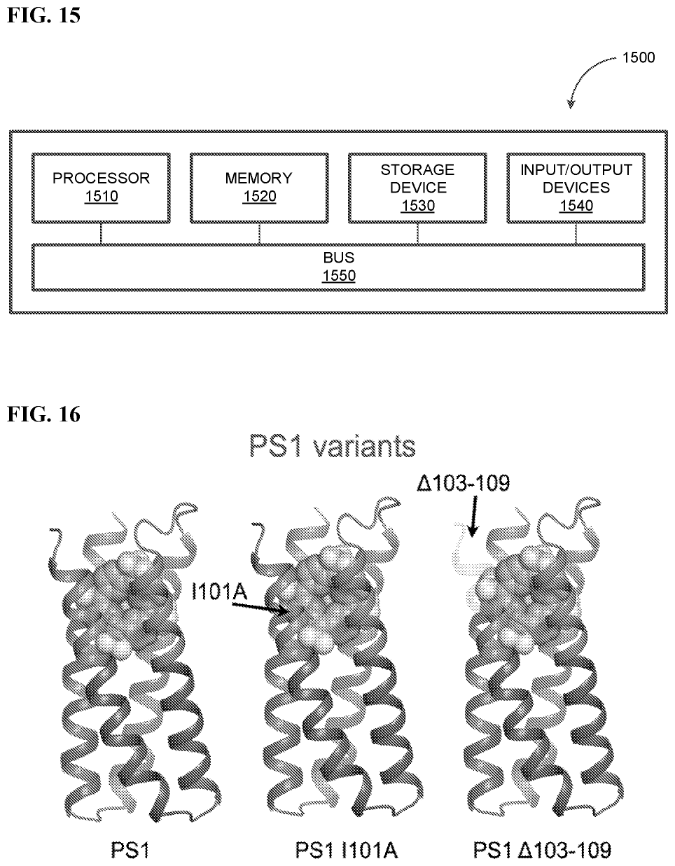

FIG. 15 depicts a block diagram illustrating a computing system 1500 consistent with implementations of the current subject matter. Referring to FIGS. 14-15, the computing system 1500 can be configured to perform the process 1400.

As shown in FIG. 15, the computing system 1500 can include a processor 1510, a memory 1520, a storage device 1530, and input/output devices 1540. The processor 1510, the memory 1520, the storage device 1530, and the input/output devices 1540 can be interconnected via a system bus 1550. The processor 1510 is capable of processing instructions for execution within the computing system 1500. Such executed instructions can implement one or more components of, for example, the database system 100 and/or the multitenant database system 200. In some example embodiments, the processor 1510 can be a single-threaded processor. Alternately, the processor 1510 can be a multi-threaded processor. The processor 1510 is capable of processing instructions stored in the memory 1520 and/or on the storage device 1530 to display graphical information for a user interface provided via the input/output device 540.

The memory 1520 is a computer readable medium such as volatile or non-volatile that stores information within the computing system 1500. The memory 1520 can store data structures representing configuration object databases, for example. The storage device 1530 is capable of providing persistent storage for the computing system 1500. The storage device 1530 can be a floppy disk device, a hard disk device, an optical disk device, or a tape device, or other suitable persistent storage means. The input/output device 540 provides input/output operations for the computing system 1500. In some example embodiments, the input/output device 540 includes a keyboard and/or pointing device. In various implementations, the input/output device 540 includes a display unit for displaying graphical user interfaces.

According to some example embodiments, the input/output device 540 can provide input/output operations for a network device. For example, the input/output device 540 can include Ethernet ports or other networking ports to communicate with one or more wired and/or wireless networks (e.g., a local area network (LAN), a wide area network (WAN), the Internet).

In some example embodiments, the computing system 1500 can be used to execute various interactive computer software applications that can be used for organization, analysis and/or storage of data in various formats. Alternatively, the computing system 1500 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities (e.g., SAP Integrated Business Planning as an add-in for a spreadsheet and/or other type of program) or can be standalone computing products and/or functionalities. Upon activation within the applications, the functionalities can be used to generate the user interface provided via the input/output device 540. The user interface can be generated and presented to a user by the computing system 1500 (e.g., on a computer screen monitor, etc.).

In an aspect is provided a system, including: at least one data processor; and at least one memory storing instructions which, when executed by the at least one data processor, result in operations including: a) identifying a set of ligand binding amino acid residues within a protein for binding to a ligand, wherein each ligand binding amino acid residue within the protein is associated with a set of ligand binding amino acid residue atomic coordinates and each atom of the ligand is associated with a set of ligand atomic coordinates; b) identifying a set of core amino acid residues within the protein that do not bind to the ligand, each core amino acid residue within the protein is associated with a set of core amino acid residue atomic coordinates; and c) optimizing the set of ligand binding amino acid residues; the set of ligand binding amino acid residue atomic coordinates; the set of core amino acid residues; and the set of core amino acid residue atomic coordinates; wherein the optimization is performed using at least an energy minimization calculation, and wherein the optimization is performed to energetically stabilize the protein.

In another aspect is provided a non-transitory computer-readable storage medium including program code, which when executed by at least one data processor, causes operations including: a) identifying a set of ligand binding amino acid residues within a protein for binding to a ligand, wherein each ligand binding amino acid residue within the protein is associated with a set of ligand binding amino acid residue atomic coordinates and each atom of the ligand is associated with a set of ligand atomic coordinates; b) identifying a set of core amino acid residues within the protein that do not bind to the ligand, each core amino acid residue within the protein is associated with a set of core amino acid residue atomic coordinates; and c) optimizing the set of ligand binding amino acid residues; the set of ligand binding amino acid residue atomic coordinates; the set of core amino acid residues; and the set of core amino acid residue atomic coordinates; wherein the optimization is performed using at least an energy minimization calculation, and wherein the optimization is performed to energetically stabilize the protein.

### IV. PROTEIN COMPOSITION

In an aspect is provided a protein sequence obtainable based on the energy minimization calculation using the method, the system, or the non-transitory computer-readable medium as described herein, including embodiments. In embodiments, the protein sequence is:

(SEQ ID NO: 1)
EFEKLRQTGDELVQAFQRLREIFDKGDDDSLEQVLEEIEELIQKHRQLEDN
RQEAADTEAAKQGDQWVQLFQRFREAIDKGDKDSLEQLLEELEQALQKIRE
LAEKKN.

In embodiments, the protein sequence is SEQ ID NO:1. In embodiments, the protein sequence is SEQ ID NO:2. In embodiments, the protein sequence is SEQ ID NO:3. In embodiments, the protein sequence is SEQ ID NO:4. In embodiments, the protein sequence is SEQ ID NO:5. In embodiments, the protein sequence is SEQ ID NO:6. In embodiments, the protein sequence is SEQ ID NO:7.

In an aspect is provided a protein, or conservatively modified variant thereof, having the sequence:

(SEQ ID NO: 1)
EFEKLRQTGDELVQAFQRLREIFDKGDDDSLEQVLEEIEELIQKHRQLEDN
RQEAADTEAAKQGDQWVQLFQRFREAIDKGDKDSLEQLLEELEQALQKIRE
LAEKKN.

In embodiments, the protein sequence is SEQ ID NO:1. In embodiments, the protein sequence is SEQ ID NO:2. In embodiments, the protein sequence is SEQ ID NO:3.

In embodiments, the protein is 99% identical to SEQ ID NO:1. In embodiments, the protein is 98% identical to SEQ ID NO:1. In embodiments, the protein is 95% identical to SEQ ID NO:1. In embodiments, the protein is 90% identical to SEQ ID NO:1. In embodiments, the protein is 85% identical to SEQ ID NO:1. In embodiments, the protein is 80% identical to SEQ ID NO:1. In embodiments, the protein is 60% identical to SEQ ID NO:1. In embodiments, the protein is about 99% identical to SEQ ID NO:1. In embodiments, the protein is about 98% identical to SEQ ID NO:1. In embodiments, the protein is about 95% identical to SEQ ID NO:1. In embodiments, the protein is about 90% identical to SEQ ID NO:1. In embodiments, the protein is about 85% identical to SEQ ID NO:1. In embodiments, the protein is about 80% identical to SEQ ID NO:1. In embodiments, the protein is about 60% identical to SEQ ID NO:1.

In embodiments, the protein is 99% identical to SEQ ID NO:2. In embodiments, the protein is 98% identical to SEQ ID NO:2. In embodiments, the protein is 95% identical to SEQ ID NO:2. In embodiments, the protein is 90% identical to SEQ ID NO:2. In embodiments, the protein is 85% identical to SEQ ID NO:2. In embodiments, the protein is 80% identical to SEQ ID NO:2. In embodiments, the protein is 60% identical to SEQ ID NO:2. In embodiments, the protein is about 99% identical to SEQ ID NO:2. In embodiments, the protein is about 98% identical to SEQ ID NO:2. In embodiments, the protein is about 95% identical to SEQ ID NO:2. In embodiments, the protein is about 90% identical to SEQ ID NO:2. In embodiments, the protein is about 85% identical to SEQ ID NO:2. In embodiments, the protein is about 80% identical to SEQ ID NO:2. In embodiments, the protein is about 60% identical to SEQ ID NO:2.

In embodiments, the protein is 99% identical to SEQ ID NO:3. In embodiments, the protein is 98% identical to SEQ ID NO:3. In embodiments, the protein is 95% identical to SEQ ID NO:3. In embodiments, the protein is 90% identical to SEQ ID NO:3. In embodiments, the protein is 85% identical to SEQ ID NO:3. In embodiments, the protein is 80% identical to SEQ ID NO:3. In embodiments, the protein is 60% identical to SEQ ID NO:3. In embodiments, the protein is about 99% identical to SEQ ID NO:3. In embodiments, the protein is about 98% identical to SEQ ID NO:3. In embodiments, the protein is about 95% identical to SEQ ID NO:3. In embodiments, the protein is about 90% identical to SEQ ID NO:3. In embodiments, the protein is about 85% identical to SEQ ID NO:3. In embodiments, the protein is about 80% identical to SEQ ID NO:3. In embodiments, the protein is about 60% identical to SEQ ID NO:3.

In embodiments, the protein is further bound to a ligand. In embodiments, the ligand is bound to the protein via a dative covalent bond. In embodiments, the ligand is a porphyrin, porphycene, rubyrin, rosarin, hexaphyrin, sapphyrin, chlorophyll, chlorin, phthalocyanine, porphyrazine, corrole, N-confused porphyrin, bacteriochlorophyll, pheophytin, texaphyrin, or related macrocyclic-based component, which is capable of binding a metal ion. In embodiments, the ligand is a detectable agent. In embodiments, the ligand is a therapeutic agent, biological agent, cytotoxic agent, magnetic resonance imaging (MRI) agent, positron emission tomography (PET) agent, radiological imaging agent, diagnostic agent, theranostic, or a photodynamic therapy (PDT) agent. In embodiments, the ligand is a catalyst. In embodiments, the catalyst catalyzes an abiological or bio-orthogonal reaction. In embodiments, the ligand is a molecule that exists within a living system.

In embodiments, the protein is 99% identical to SEQ ID NO:8. In embodiments, the protein is 98% identical to SEQ ID NO:8. In embodiments, the protein is 95% identical to SEQ ID NO:8. In embodiments, the protein is 90% identical to SEQ ID NO:8. In embodiments, the protein is 85% identical to SEQ ID NO:8. In embodiments, the protein is 80% identical to SEQ ID NO:8. In embodiments, the protein is 60% identical to SEQ ID NO:8. In embodiments, the protein is about 99% identical to SEQ ID NO:8. In embodiments, the protein is about 98% identical to SEQ ID NO:8. In embodiments, the protein is about 95% identical to SEQ ID NO:8.

In embodiments, the protein is about 90% identical to SEQ ID NO:8. In embodiments, the protein is about 85% identical to SEQ ID NO:8. In embodiments, the protein is about 80% identical to SEQ ID NO:8. In embodiments, the protein is about 60% identical to SEQ ID NO:8.

Informal Sequence Listing:

(SEQ ID NO: 1)
EFEKLRQTGDELVQAFQRLREIFDKGDDDSLEQVLEEIEELIQKHRQLFDN
RQEAADTEAAKQGDQWVQLFQRFREAIDKGDKDSLEQLLEELEQALQKIRE
LAEKKN.
(SEQ ID NO: 2)
SEFEKLRQTGDELVQAFQRLREIFDKGDDDSLEQVLEEIEELIQKHRQLFD
NRQEAADTEAAKQGDQWVQLFQRFREAIDKGDKDSLEQLLEELEQALQKIR
ELAEKKN.
(SEQ ID NO: 3)
CATATGCATCACCATCACCATCACGAAAACCTGTATTTTCAGAGCGAATTC
GAAAAACTGCGTCAAACCGGCGACGAACTGGTGCAGGCATTTCAACGTCTG
CGCGAAATTTTCGATAAAGGTGATGACGATAGTCTGGAACAGGTTCTGGAA
GAAATTGAAGAACTGATCCAGAAACATCGTCAACTGTTTGACAATCGCCAG
GAAGCGGCCGATACGGAAGCAGCTAAACAGGGCGACCAATGGGTCCAGCTG
TTTCAACGTTTCCGCGAAGCCATTGATAAAGGTGACAAAGATAGCCTGGAA
CAGCTGCTGGAAGAACTGGAACAGGCGCTGCAAAAAATCCGCGAACTGGCC
GAAAAGAAAAACTAAGGATCC
(SEQ ID NO: 4)
MHHHHHHENLYFQ/SEFEKLRQTGDELVQAFQRLREIFDKGDDDSLEQVLE
EIEELIQKHRQLFDNRQEAADTEAAKQGDQWVQLFQRFREAIDKGDKDSLE
QLLEELEQALQKIRELAEKKN
(SEQ ID NO: 5)
LGLVAFLIFGLVLILIHLFAAGWVFFAILLLLALILA
(SEQ ID NO: 6)
LGIILLLAIGLILLAFHLFFAGWLFIAILLFSGIILA
(SEQ ID NO: 7)
SEFEKLRQTGDEIIQLLQRLREAIDKGDDDSLEQILEELEEAFQKHRQLFE
NRQEAADTEFAKQGDQWLQLFQRIREAIDKGDKDSLEQLFEESEQGIQKIR
ELAEKKN
(SEQ ID NO: 8)
EFEKLRQTGDELVQAFQRLREIFDKGDDDSLEQVLEEIEELIQKHRQLFDN
RQEAADTEAAKQGDQWVQLFQRFREAIDKGDKDSLEQLLEELEQALQKIR

### V. EMBODIMENTS

### Embodiment 1

A computer-implemented method, comprising: (a) identifying a set of ligand binding amino acid residues within a protein for binding to a ligand, wherein each ligand binding amino acid residue within said protein is associated with a set of ligand binding amino acid residue atomic coordinates and each atom of said ligand is associated with a set of ligand atomic coordinates; (b) identifying a set of core amino acid residues within said protein that do not bind to said ligand, each core amino acid residue within said protein is associated with a set of core amino acid residue atomic coordinates; and (c) optimizing: said set of ligand binding amino acid residues; said set of ligand binding amino acid residue atomic coordinates; said set of core amino acid residues; and said set of core amino acid residue atomic coordinates; wherein the optimization is performed using at least an energy minimization calculation, and wherein the optimization is performed to energetically stabilize said protein.

### Embodiment 2

The method of embodiment 1, wherein step c) comprises simultaneously optimizing: said set of ligand binding amino acid residues; said set of ligand binding amino acid residue atomic coordinates; said set of core amino acid residues; and said set of core amino acid residue atomic coordinates.

### Embodiment 3

The method of embodiment 1, wherein the energy minimization calculation comprises a molecular mechanics function, a structural bioinformatics function, an amino acid sidechain packing function, a protein radius of gyration function, or a combination thereof.

### Embodiment 4

The method of embodiment 1, wherein the core amino acids are at least 75% inaccessible to a 1.8 Å spherical probe.

### Embodiment 5

The method of embodiment 1, wherein said set of core amino acids comprises at least six amino acid residues.

### Embodiment 6

The method of any one of embodiments 1 to 5, wherein the optimizing comprises fixing an atomic coordinate of at least one ligand binding amino acid residue atomic coordinate; fixing an atomic coordinate of at least one ligand atomic coordinate; prohibiting introduction of an additional amino acid residue into the set of ligand binding amino acid residues; or prohibiting the deletion of an amino acid residue from the set of ligand binding amino acid residues.

### Embodiment 7

The method of any one of embodiments 1 to 5, wherein the optimizing comprises fixing at least one atomic coordinate of the ligand atomic coordinates.

### Embodiment 8

The method of any one of embodiments 1 to 7, wherein the energy minimization calculation comprises a penalty function.

### Embodiment 9

The method of any one of embodiments 1 to 8, wherein the optimizing does not comprise fixing at least one atomic coordinate of at least one core amino acid residue atomic coordinates.

### Embodiment 10

The method of any one of embodiments 1 to 8, wherein the optimizing comprises introducing an additional ligand binding amino acid residue into the set of ligand binding amino acid residues, deleting a ligand binding amino acid residue from the set of ligand binding amino acid residues, a geometric transformation of at least one atomic coordinate of the ligand binding amino acid residue atomic coordinates.

### Embodiment 11

The method of embodiment 10, wherein the geometric transformation comprises a translation or a rotation of at least one atomic coordinate of the ligand binding amino acid residue atomic coordinates.

### Embodiment 12

The method of any one of embodiments 1 to 11, wherein the optimizing comprises a geometric transformation of at least one atomic coordinate of the core amino acid residue atomic coordinates.

### Embodiment 13

The method of any one of embodiments 10 to 12, wherein the geometric transformation of at least one atomic coordinate comprises no greater than a 6 Å displacement of any atomic coordinate.

### Embodiment 14

The method of any one of embodiments 10 to 12, wherein the geometric transformation of at least one atomic coordinate comprises no greater than a 3 Å displacement of any atomic coordinate.

### Embodiment 15

The method of any one of embodiments 1 to 14, wherein the optimizing comprises an iterative or heuristic algorithm.

### Embodiment 16

The method of any one of embodiments 1 to 14, wherein the optimizing comprises a simplex algorithm, memetic algorithm, differential evolution algorithm, evolutionary algorithm, genetic algorithm, tabu algorithm, particle swarm algorithm, or stimulated annealing algorithm.

### Embodiment 17

The method of any one of embodiments 1 to 14, wherein the optimizing comprises a Monte Carlo sampling algorithm, dead-end elimination algorithm, branch and bound algorithm, or a pruning algorithm.

### Embodiment 18

The method of any one of embodiments 1 to 17, wherein the ligand is a porphyrin, porphycene, rubyrin, rosarin, hexaphyrin, sapphyrin, chlorophyll, chlorin, phthalocyanine, porphyrazine, corrole, N-confused porphyrin, bacteriochlorophyll, pheophytin, texaphyrin, or related macrocyclic-based component, that is capable of binding a metal ion.

### Embodiment 19

The method of any one of embodiments 1 to 17, wherein the ligand is a detectable agent.

### Embodiment 20

The method of any one of embodiments 1 to 17, wherein the ligand is a therapeutic agent, biological agent, cytotoxic agent, magnetic resonance imaging (MRI) agent, positron emission tomography (PET) agent, radiological imaging agent, diagnostic agent, theranostic, or a photodynamic therapy (PDT) agent.

### Embodiment 21

The method of any one of embodiments 1 to 17, wherein the ligand is a catalyst.

### Embodiment 22

The method of any one of embodiments 1 to 17, wherein the catalyst catalyzes an abiological or bio-orthogonal reaction.

### Embodiment 23

The method of any one of embodiments 1 to 17, wherein the ligand is a molecule that exists within a living system.

### Embodiment 24

A system, comprising: at least one data processor; and at least one memory storing instructions which, when executed by the at least one data processor, result in operations comprising: (a) identifying a set of ligand binding amino acid residues within a protein for binding to a ligand, wherein each ligand binding amino acid residue within said protein is associated with a set of ligand binding amino acid residue atomic coordinates and each atom of said ligand is associated with a set of ligand atomic coordinates; (b) identifying a set of core amino acid residues within said protein that do not bind to said ligand, each core amino acid residue within said protein is associated with a set of core amino acid residue atomic coordinates; and (c) optimizing: said set of ligand binding amino acid residues; said set of ligand binding amino acid residue atomic coordinates; said set of core amino acid residues; and said set of core amino acid residue atomic coordinates; wherein the optimization is performed using at least an energy minimization calculation, and wherein the optimization is performed to energetically stabilize said protein.

### Embodiment 25

The system of embodiment 24, wherein the energy minimization calculation comprises functions from molecular mechanics, functions from structural bioinformatics, amino acid sidechain packing functions, protein radius of gyration functions, or a combination thereof.

### Embodiment 26

The system of embodiment 24, wherein the core amino acids are at least 75% inaccessible to a 1.8 Å spherical probe.

### Embodiment 27

The system of embodiment 24, wherein said set of core amino acids comprise at least six amino acid residues.

### Embodiment 28

The system of any one of embodiments 24 to 27, wherein the optimizing comprises fixing an atomic coordinate of at least one ligand binding amino acid residue atomic coordinate; fixing an atomic coordinate of at least one ligand atomic coordinate; prohibiting introduction of an additional amino acid residue into the set of ligand binding amino acid residues; or prohibiting the deletion of an amino acid residue from the set of ligand binding amino acid residues.

### Embodiment 29

The system of any one of embodiments 24 to 28, wherein the optimizing comprises fixing at least one atomic coordinate of the ligand atomic coordinates.

### Embodiment 30

The system of any one of embodiments 24 to 29, wherein the energy minimization calculation comprises a penalty function.

### Embodiment 31

The system of any one of embodiments 24 to 30, wherein the optimizing does not comprise fixing at least one atomic coordinate of at least one core amino acid residue atomic coordinates.

### Embodiment 32

The system of any one of embodiments 24 to 31, wherein the optimizing comprises introducing an additional ligand binding amino acid residue into the set of ligand binding amino acid residues, deleting a ligand binding amino acid residue from the set of ligand binding amino acid residues, a geometric transformation of at least one atomic coordinate of the ligand binding amino acid residue atomic coordinates.

### Embodiment 33

The method of embodiment 32, wherein the geometric transformation comprises a translation or a rotation of at least one atomic coordinate of the ligand binding amino acid residue atomic coordinates.

### Embodiment 34

The system of any one of embodiments 24 to 33, wherein the optimizing comprises a geometric transformation of at least one atomic coordinate of the core amino acid residue atomic coordinates.

### Embodiment 35

The system of any one of embodiments 24 to 34, wherein the geometric transformation of at least one atomic coordinate comprises no greater than a 6 Å displacement of any atomic coordinate.

### Embodiment 36

The system of any one of embodiments 24 to 34, wherein the geometric transformation of at least one atomic coordinate comprises no greater than a 3 Å displacement of any atomic coordinate.

### Embodiment 37

The system of any one of embodiments 24 to 36, wherein the optimizing comprises an iterative or heuristic algorithm.

### Embodiment 38

The system of any one of embodiments 24 to 36, wherein the optimizing comprises a simplex algorithm, memetic algorithm, differential evolution algorithm, evolutionary algorithm, genetic algorithm, tabu algorithm, particle swarm algorithm, or stimulated annealing algorithm.

### Embodiment 39

The system of any one of embodiments 24 to 36, wherein the optimizing comprises a Monte Carlo sampling algorithm, dead-end elimination algorithm, branch and bound algorithm, or a pruning algorithm.

### Embodiment 40

A non-transitory computer-readable storage medium including program code, which when executed by at least one data processor, causes operations comprising: (a) identifying a set of ligand binding amino acid residues within a protein for binding to a ligand, wherein each ligand binding amino acid residue within said protein is associated with a set of ligand binding amino acid residue atomic coordinates and each atom of said ligand is associated with a set of ligand atomic coordinates; (b) identifying a set of core amino acid residues within said protein that do not bind to said ligand, each core amino acid residue within said protein is associated with a set of core amino acid residue atomic coordinates; and (c) optimizing: said set of ligand binding amino acid residues; said set of ligand binding amino acid residue atomic coordinates; said set of core amino acid residues; and said set of core amino acid residue atomic coordinates; wherein the optimization is performed using at least an energy minimization calculation, and wherein the optimization is performed to energetically stabilize said protein.

### Embodiment 41

A protein sequence obtainable based on the energy minimization calculation using the method of any of embodiments 1 to 23, the system of any of embodiments 24 to 39, or the non-transitory computer-readable medium of embodiment 40.

### Embodiment 42

A protein, or conservatively modified variant thereof, having the sequence SEQ ID NO:1.

### Embodiment 43

The protein of embodiment 42, wherein the protein is 90% identical to SEQ ID NO:1.

### Embodiment 44

The protein of embodiment 42, bound to a ligand.

### Embodiment 45

The protein of embodiment 42, wherein the ligand is bound to the protein via a dative covalent bond.

### Embodiment 46

The protein of embodiment 44, wherein the ligand is a porphyrin, porphycene, rubyrin, rosarin, hexaphyrin, sapphyrin, chlorophyll, chlorin, phthalocyanine, porphyrazine, corrole, N-confused porphyrin, bacteriochlorophyll, pheophytin, texaphyrin, or related macrocyclic-based component, that is capable of binding a metal ion.

### Embodiment 47

The protein of embodiment 44, wherein the ligand is a detectable agent.

### Embodiment 48

The protein of embodiment 44, wherein the ligand is a therapeutic agent, biological agent, cytotoxic agent, magnetic resonance imaging (Mill) agent, positron emission tomography (PET) agent, radiological imaging agent, diagnostic agent, theranostic, or a photodynamic therapy (PDT) agent.

### Embodiment 49

The protein of embodiment 44, wherein the ligand is a catalyst.

### Embodiment 50

The protein of embodiment 44, wherein the catalyst catalyzes an abiological or bio-orthogonal reaction.

### Embodiment 51

The protein of embodiment 44, wherein the ligand is a molecule that exists within a living system.

### EXAMPLES

### Example 1—Strategy for Designing Hyperstable, Non-Natural Protein-Cofactor Complexes with Sub-Δ Accuracy

While the de novo design of proteins has seen many successes¹⁻¹², no small molecule ligand- or organic cofactor-binding protein has been designed entirely from first principles to achieve i) a unique structure and ii) a predetermined binding-site geometry with sub-Å accuracy. Such achievements are prerequisites for the design of proteins that control and enable complex reaction trajectories, where the relative placements of cofactors, substrates, and protein side chains must be established within the length scale of a chemical bond. Here, we design a small molecule-binding protein based on the concept that the entire protein contributes to establishing the binding geometry of a ligand¹³⁻¹⁶. Mutational studies of natural ligand-binding proteins have highlighted the counter-intuitive importance of distant amino acids (10-20 Å from the binding site) on binding affinity, which work in concert with first-shell amino acids surrounding the bound ligand¹³⁻¹⁶. We implement this concept for the first time in de novo protein design. Hence, what are traditionally considered as separate sectors—the hydrophobic core and ligand-binding site—we treat as an inseparable unit. We utilize flexible backbone sequence design of a parametrically defined protein template to simultaneously pack the protein interior both proximal to and remote from the ligand-binding site. Thus, tight interdigitation of core side chains quite removed from the binding site structurally restrains the first- and second-shell packing around the ligand. We apply this principle to the decades-old problem of structural non-uniqueness in de novo-designed heme-binding proteins¹⁷. We designed a novel protein, PS1, which binds a highly electron-deficient, non-natural porphyrin at temperatures up to 100° C. The high-resolution structure of holo-PS1 is in sub-A agreement with the design. The structure of apo-PS1 retains the remote core packing of the holo, predisposing a flexible binding region for the desired ligand-binding geometry. Our results illustrate the unification of core packing and binding site definition as a fundamental principle of ligand-binding protein design.

Recent successes in the field of de novo design of coiled coils^{3,7} and metalloproteins^{4,8-10} are encouraging, but so far have not translated to more complex cofactors. In fact, attempts at computational design of novel small molecule ligand-binding proteins have been limited in number and generally focused on changing only the binding site of natural proteins, leaving the core of the protein intact^{18,19}. For example, the binding site of a natural scaffold was computationally redesigned to bind a hydrophobic organic ligand but required multiple rounds of mutagenesis and experimental selection using yeast display¹⁸. At the other extreme, de novo heme-binding helical bundle proteins have been designed entirely from first principles (17, 20), but these “maquettes” have evaded structural determination, largely due to aggregation or their dynamical properties^{17,21,22}. With the exception of short, covalently linked peptide-heme complexes²³, the only structure of a de novo heme-binding protein was solved for an apo-protein, which showed a hydrophobically collapsed binding site with no space for binding heme^{21,24}. The lack of precise, predictive three-dimensional models of heme-binding maquettes, coupled with the failure to determine high-resolution structures, has limited their utility, although maquettes have elucidated electrostatic roles for tuning redox potentials of donors/acceptors in electron-transfer reactions²⁰. An iterative trial-and-error approach has been shown to incrementally improve NMR spectra of maquette proteins²⁵, and may ultimately lead to the determination of three-dimensional structures; however, a robust computational method is needed to deliver precisely predetermined structures with sub-A accuracy.

Our own work has focused on the development of computational design of cofactor-binding proteins²⁶⁻²⁸ with atomic-level accuracy. We used a step-wise strategy in which we first employed a mathematical parameterization of an antiparallel coiled coil to construct a rigid binding site, then, in a separate calculation, introduced side chain packing constrained by this rigid backbone²⁶⁻²⁸. This approach resulted in de novo porphyrin-binding proteins with the desired tertiary structure and ligand-binding stoichiometry, but not of sufficient conformational uniqueness to yield a high-resolution structure.

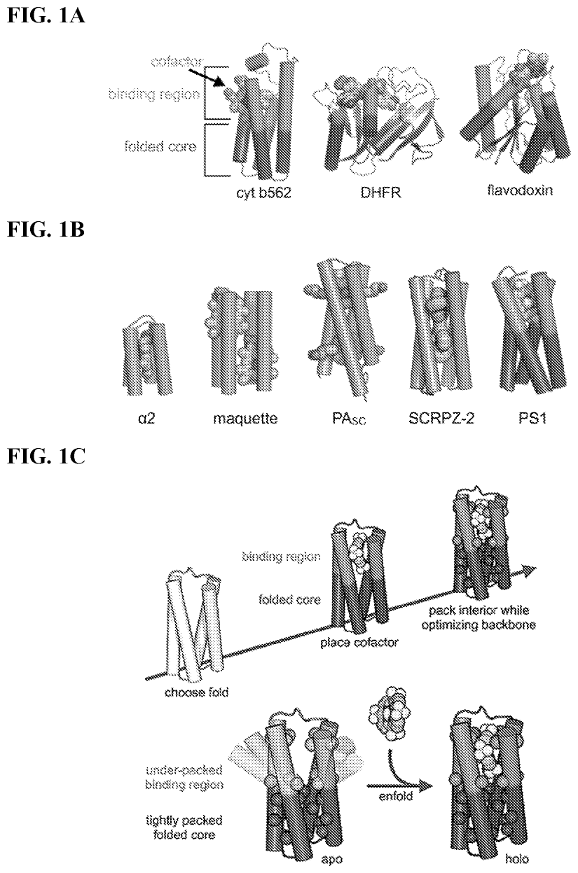

A body of work with natural proteins¹³⁻¹⁶ has shown that side chain packing quite distant from the binding site can propagate to significantly affect ligand binding, catalysis, and allosteric regulation. Thus, the entire hydrophobic core—even residues 20 Å away from the binding site—should be considered as an essential extension of the primary and secondary shell interactions with the ligand. We noted that, unlike natural proteins (FIG. 1A), previous de novo designed cofactor-binding proteins lack an extensive, well-defined apolar core. Instead, their interior packing is dominated by interactions with one or more porphyrins or multi-functional cofactors that span the length of the bundle (FIG. 1B). Where a cofactor-free core was included²⁹, the core was not computationally designed, and high-resolution structures were not determined. Here, we purposefully include a folded core remote from the ligand-binding site and optimize its sequence and structure in concert with the binding site to ensure appropriate coupling (FIG. 1C). As compared to earlier computational design of ligand-binding proteins^{11,18} our approach differs by: 1) beginning with a mathematically parameterized backbone rather than a natural protein; 2) applying flexible backbone design to the entire backbone as well as sequence design to all interior and substrate-binding sites rather than just the first and second-shell contacting residues; 3) not relying on screening of large numbers of designs or genetic selections to achieve the desired outcome.

Protein Design.

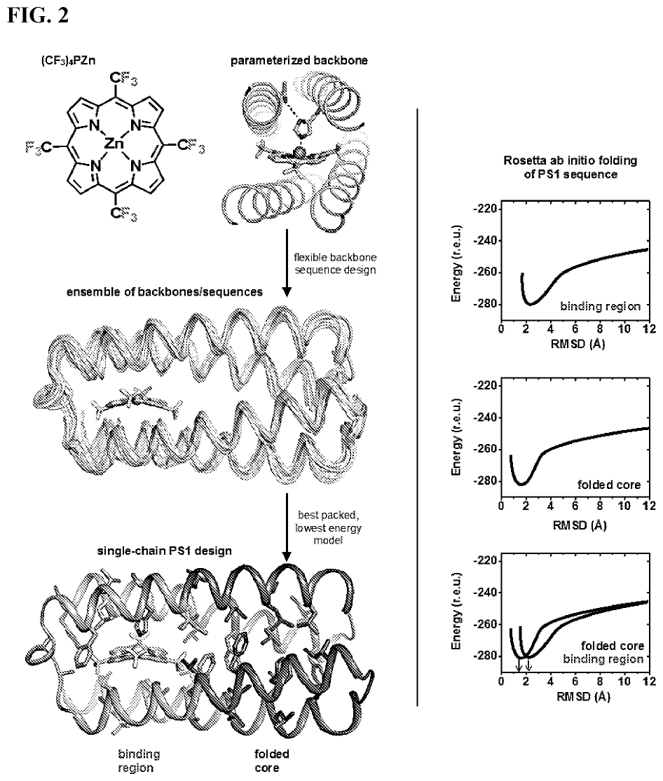

The design of PS1 (Porphyrin-binding Sequence 1) began with the previously parameterized backbone from the de novo designed protein SCRPZ-2²⁸, a protein that bound an extended porphinato(metal)-polypyridyl(metal) cofactor (FIG. 1B). The backbone of SCRPZ-2 and its di-porphyrin-binding predecessors^{26,30} was designed with a simple equation defining a D₂-symmetrical antiparallel coiled coil³¹. The parameters were adjusted to position a single His ligand to receive a second-shell hydrogen bond with Thr from a neighboring helix (see FIG. 2). Side chains in the vicinity of the binding site were computationally designed to stabilize the asymmetric ligand environment while maintaining a rigid symmetrical backbone. Interhelical loops were then chosen following previously defined geometric principles^{26,28,32}. Although SCRPZ-2 bound to its desired cofactor, its NMR spectra was not as well dispersed as those for natural heme-containing proteins, and it lacked a cooperatively folded core.

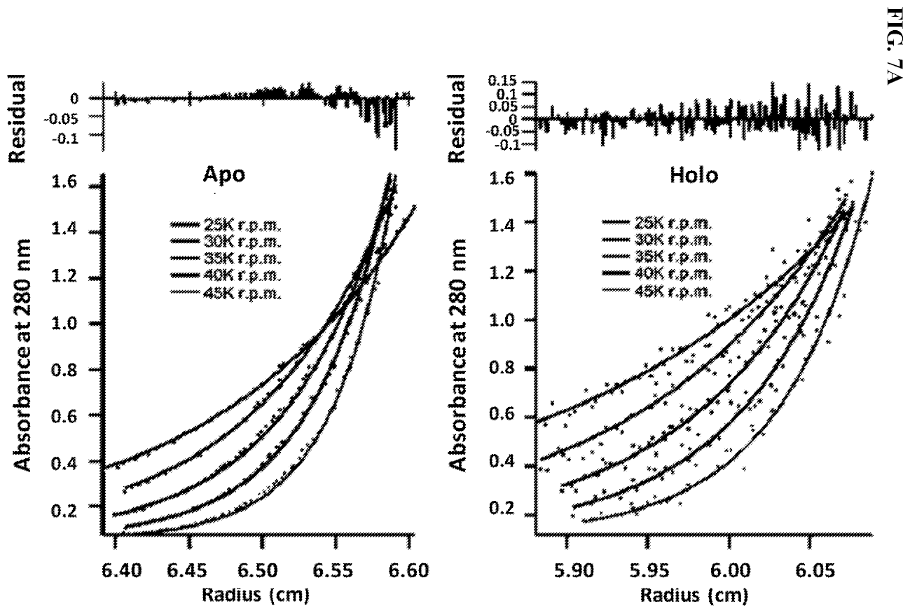

We used the parameterized backbone of SCRPZ-2 as a starting point for design of a protein that binds a much smaller abiological porphyrin (CF₃)₄PZn ([5,10,15,20-tetrakis(trifluoromethyl)porphinato]Zn²⁺) (FIG. 2)³³, a powerful photo-oxidant with an excited-state reduction potential similar to the ground-state reduction potential of the oxidized special pair of chlorophylls in photosystem II of green plants³⁴. The reduced size of the (CF₃)₄PZn cofactor provided space for a hydrophobic core in what was formerly occupied by the large, bulky metal-polypyridyl group. We manually docked (CF₃)₄PZn in the porphyrin-binding site (FIG. 2) and used Backrub within Rosetta³⁵ to sample small structural changes of the parameterized backbone; we then employed alternating loops of fixed backbone sequence design and backbone/sidechain minimization. The models were assessed for packing of the porphyrin as well as the core. To isolate effects of introducing a well-defined hydrophobic core, we allowed sequence changes only in the protein interior and cofactor-binding site, keeping the identities of most solvent-exposed and loop residues fixed from that of SCRPZ-2. The final sequence of PS1 shares no similarity with any known natural protein (BLAST E value<0.06 against the non-redundant protein sequence database nr). Although the final backbone model of PS1 differed by only 1 Å root mean square deviation (RMSD) from the initial parameterized backbone of SCRPZ-2, fully 70% of the interior residues were changed from SCRPZ-2, and half of those retained were predicted to adopt different rotamers (FIG. 6).

Biophysical Characterization of PS1.

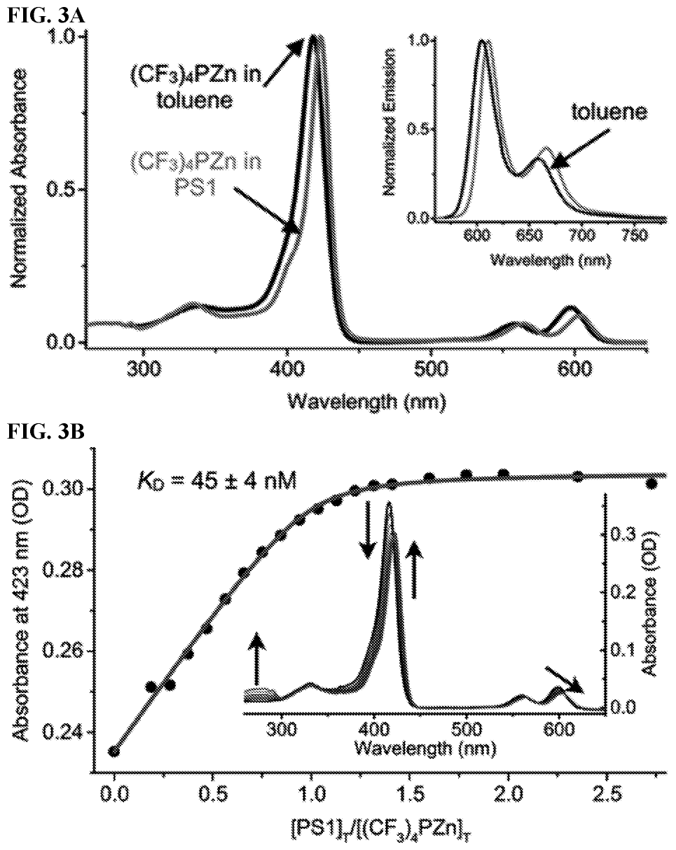

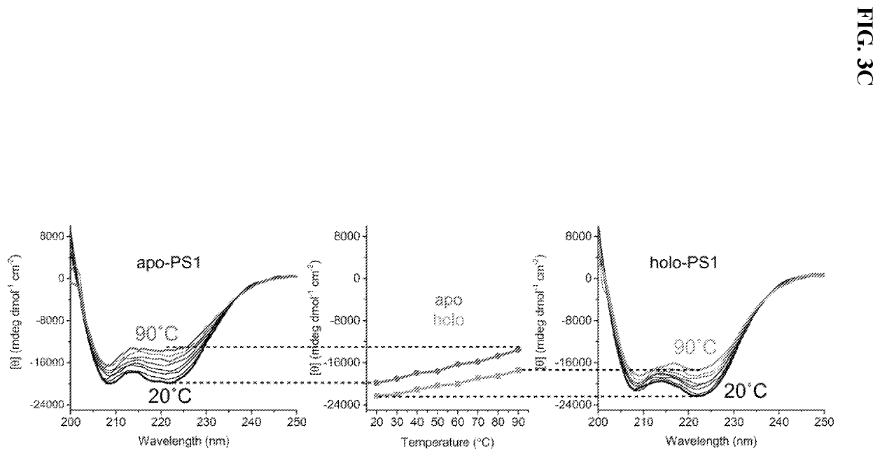

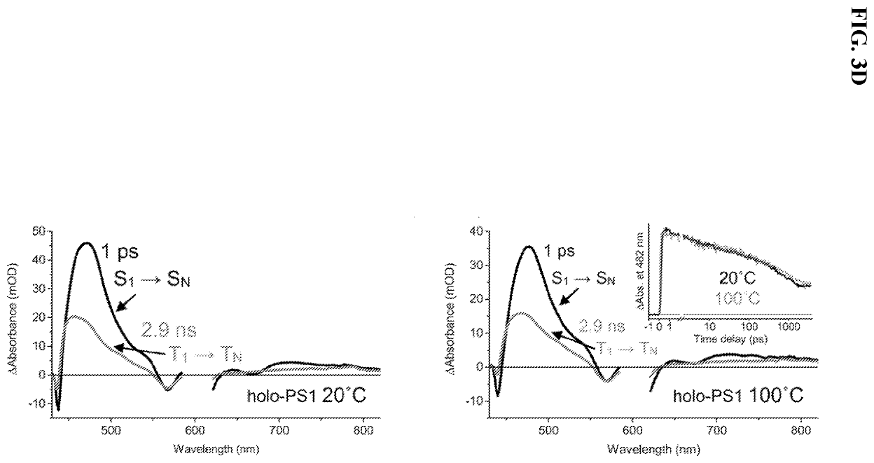

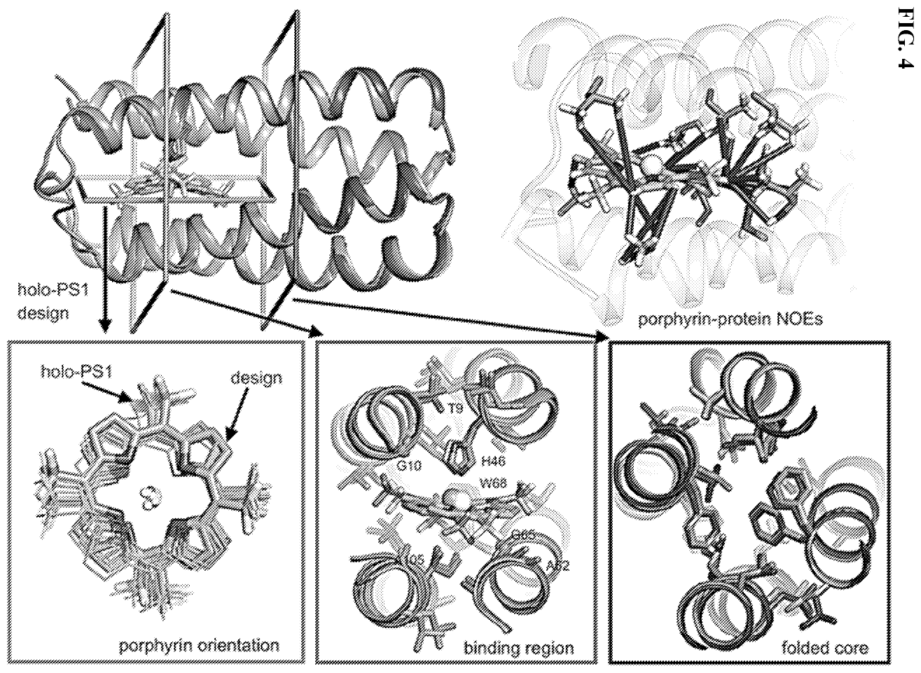

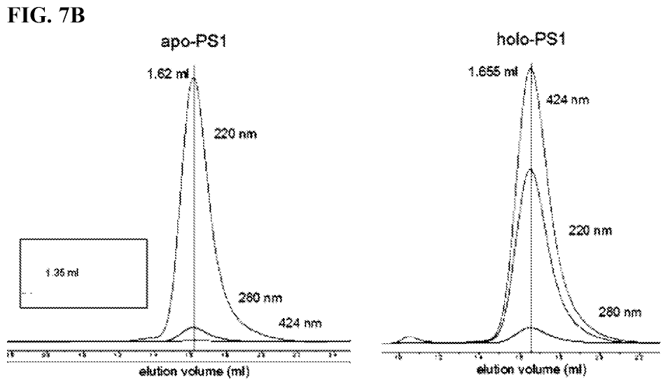

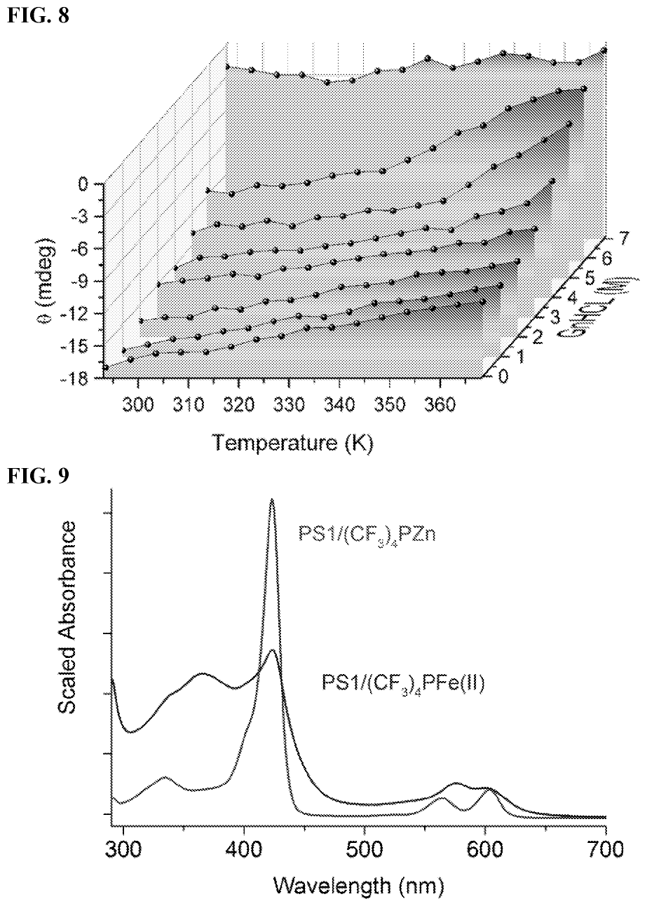

PS1 is monomeric (FIGS. 7A-7B) and binds the water-insoluble cofactor, (CF₃)₄PZn, forming highly thermostable complexes (extrapolated Tₘ>120° C., FIG. 3c and Fig. S3) that are stable for over a year. The complex forms within seconds of adding (CF₃)₄PZn from organic solution to aqueous PS1, suggesting a small kinetic barrier for assembly (FIG. 3A). A tight dissociation constant of binding, K_{D}=45 nM, was measured under conditions where the water-insoluble porphyrin was solubilized with 1% w/v octylglucopyranoside detergent (FIG. 3B). PS1 also binds the ferrous iron-derivative of the porphyrin, (CF₃)₄PFe (FIG. 9), despite the abysmal solubility in water of this cofactor. Loading of PS1 with (CF₃)₄PFe suggests that the protein could also be used as a platform for engineering ground-state redox chemistry, as (CF₃)₄PFe is an electron-deficient (porphinato)metal complex capable of molecular oxygen activation for alkane hydroxylation and alkene epoxidation³⁶.

Time-resolved transient absorption spectroscopy showed that protein/(CF₃)₄PZn interactions are preserved even at near-boiling temperatures where the protein retains its native structure (FIG. 3D). The excited-state spectra and dynamics of (CF₃)₄PZn within holo-PS1 at 21 and 100° C. are indistinguishable, which indicates that the protein does not detectably perturb the porphyrin molecular framework—intersystem crossing rates of electronically excited porphyrins are known to be sensitive to temperature and environment³⁷. Furthermore, these data indicate that encapsulation of (CF₃)₄PZn in the binding site of PS1 shields the porphyrin from nucleophilic attack that would otherwise occur in water, especially at high temperatures, i.e. the protein safeguards the porphyrin against a wasteful, degradative, photochemical side reaction. Thus, PS1 effectively stabilizes an extraordinarily insoluble cofactor in aqueous solution, even at temperatures considered extreme for hyperthermophiles.

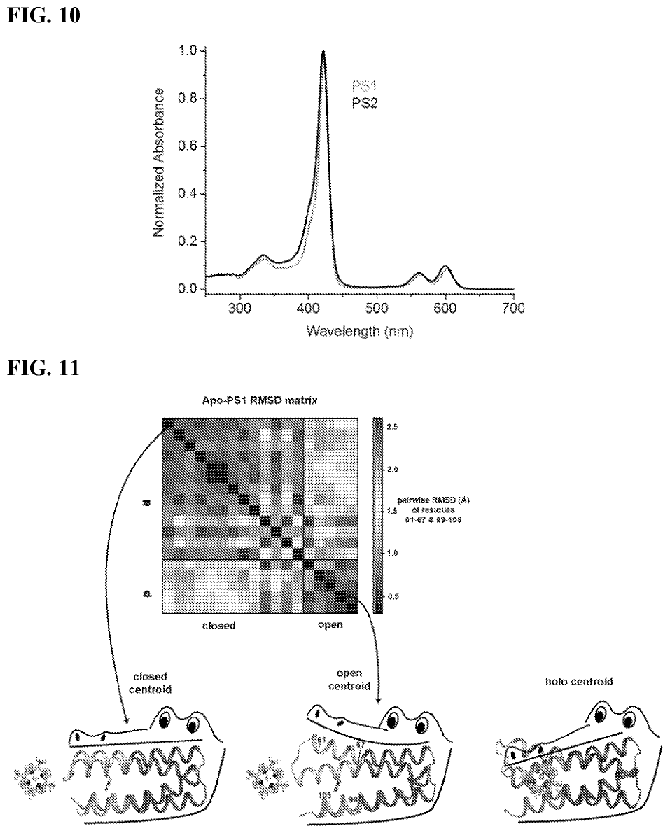

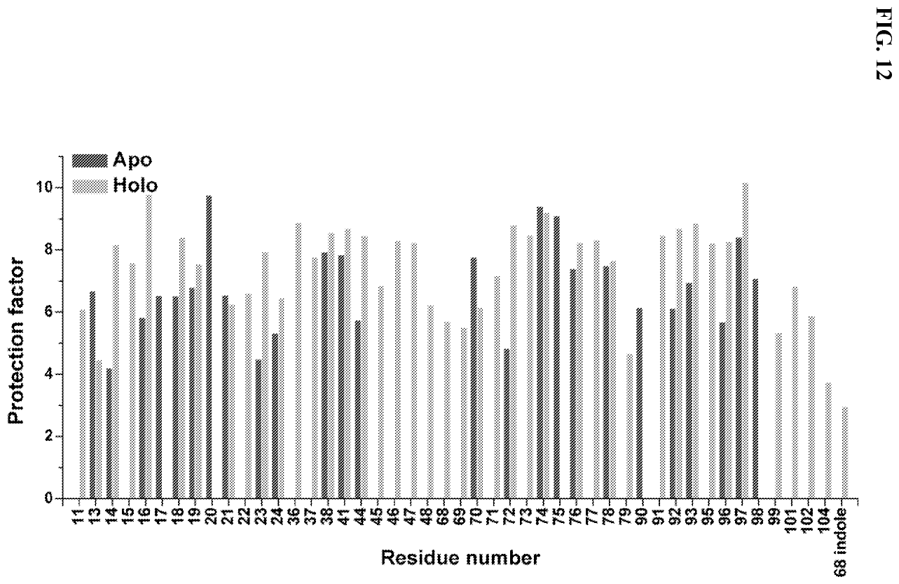

We also examined another high-scoring sequence (named PS2) of the design process, with a hydrophobic core unique from PS1, which was expressed, purified, and tested for binding to (CF₃)₄PZn. Electronic absorption spectra of holo-PS2 shows narrow absorption bands similar to those evinced by holo-PS1 (FIG. 10), strongly suggesting that these designs analogously enfold the porphyrin in a unique binding environment.

Structural Characterization of Holo-PS1.

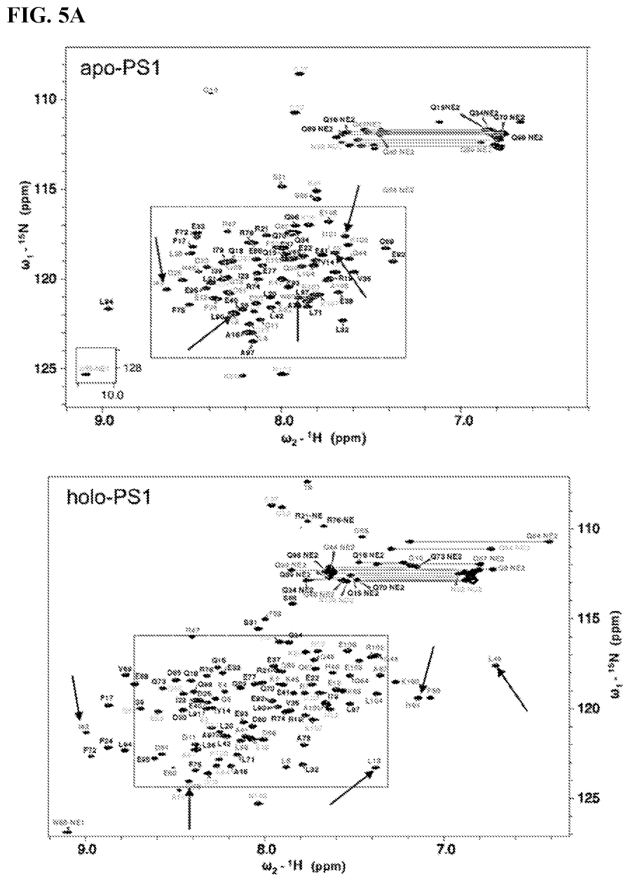

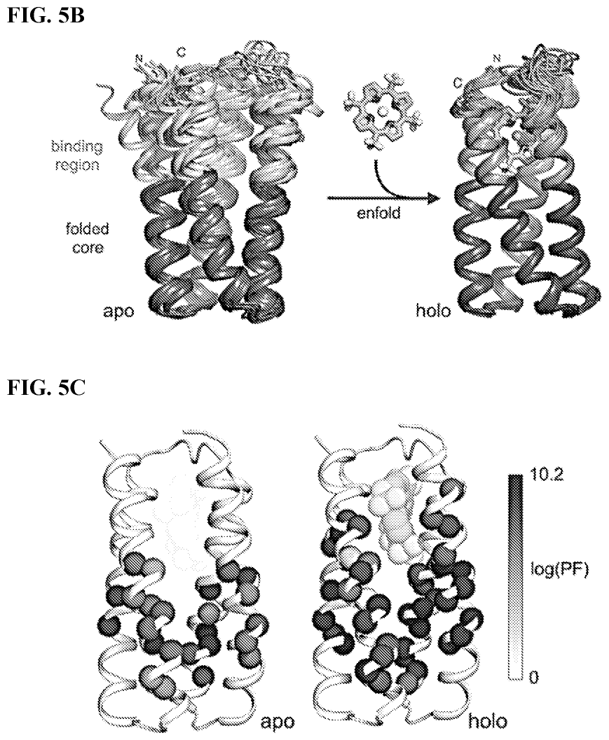

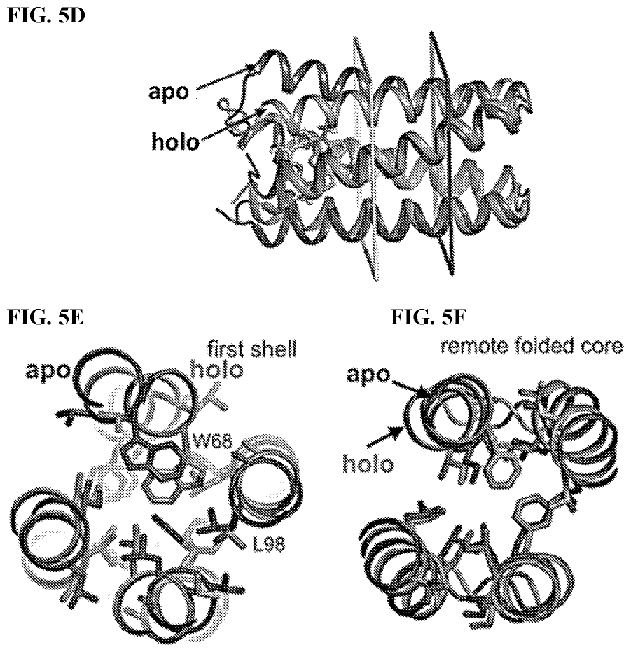

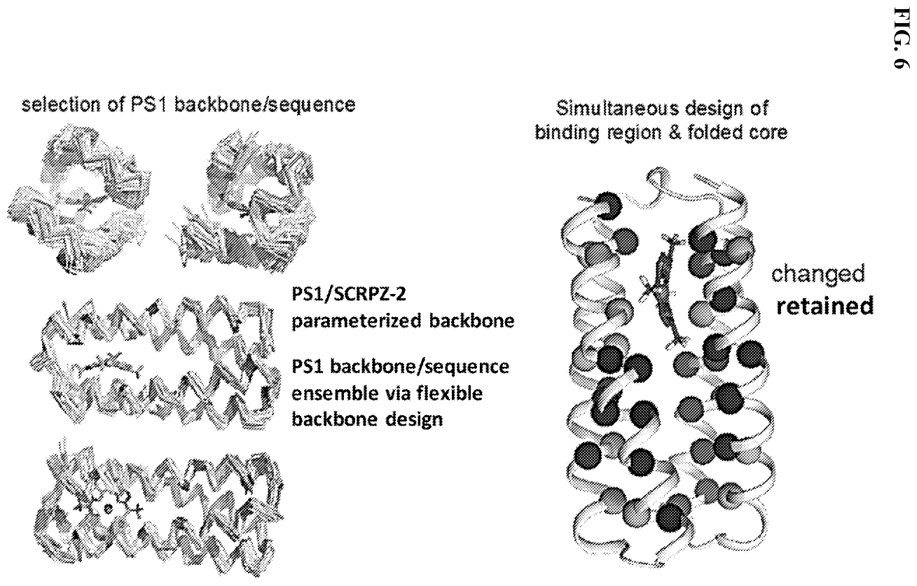

An exceptionally well-resolved NMR structural ensemble of holo-PS1 (FIGS. 4 and 5) was computed using 19 nuclear Overhauser effects (NOEs) per residue and nearly complete ¹D_{NH} residual dipolar coupling restraints. The backbone is in excellent agreement with the design (0.8±0.1 Å helical backbone RMSD), and core residues each populate a single rotamer state, almost all in agreement with the design (FIGS. 4A,D,E). While the PS1 design was selected based in part on its featuring an abundance of high-probability rotamer states of core residues, two low-probability rotamers were present in the designed core of PS1: one, Leu98, in the first-shell of the binding site, and the other, Leu19, in the remote folded core. Binding of the porphyrin forces Leu98 to adopt this low-probability rotamer, which is not present in the apo-protein (see FIG. 5E), whereas Leu19 adopts a more probable rotamer in both the holo- and apo-proteins. Trp68, fit snuggly between two CF₃ groups of the porphyrin, can also be seen to adopt its predicted rotamer upon binding of the porphyrin, driving a unique conformation of the cofactor within the binding site.

The location and orientation of the porphyrin within PS1 was determined by an exceptional number of porphyrin-protein NOEs (26 porphyrin-protein NOEs were used in the structural refinement, FIG. 4). Most importantly, the observed orientation of the cofactor is exactly as designed, within the precision of the NMR structure (FIG. 4). (CF₃)₄PZn was only displaced in its binding site relative to its predetermined orientation by an average translation (0.4 Å) half the size of a covalent C—H bond, and by a small average rotation (11°) within the porphyrin plane.

Ab Initio Folding Predictions and NMR Structure of Apo-PS1.

Ab initio folding³⁸ simulations of the apo-PS1 sequence predict a bipartite structure with a conformationally unique folded core, which closely resembles the core of holo-PS1, and a more flexible cofactor-binding region (FIG. 2). Significantly, hydrophobic collapse in the binding region is avoided, because it contains a polar His and also is rich in small Ala and Gly side chains (FIG. 4) to specifically associate with the face of the porphyrin ring, rather than the large hydrophobic residues used to stabilize hemes in maquettes. Thus, “negative design” in PS1 is implicitly achieved through the construction of a relatively polar cofactor-binding site, which creates a cofactor-shaped void in the apo-protein.

The NMR structure of apo-PS1 was also solved (FIG. 5), and the structural ensemble shows a folded core highly similar to that of holo-PS1. This finding indicates that the folded core both predisposes and anchors the flexible binding region for productive binding of the ligand. The binding region is more dynamic in apo-PS1, which contains two clusters of structures, open and closed. The open conformation likely facilitates binding of the large cofactor, but there is room for water to penetrate into the unoccupied binding site in both conformations.

Dynamics and Structural Comparisons of Apo-Vs Holo-PS1.

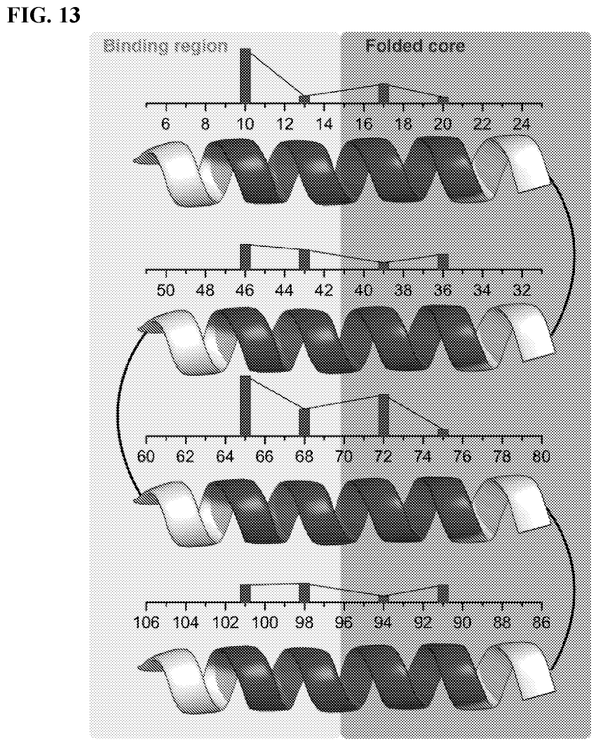

Solvent hydrogen-deuterium exchange (HDX) experiments and molecular dynamics simulations of apo-PS1 also show a gradient in conformational stability between the apolar core and the binding site of apo-PS1 (FIG. 5C, FIGS. 12 and 13). The backbone surrounding the apolar core of both holo- and apo-PS1 is highly protected from exchange, an important characteristic of cooperatively folded native proteins. The protected region extends into the porphyrin-binding site in the holo-protein but not in the apo-structure (FIG. 5C). The increased protection in the binding site of holo-PS1 is seen at both solvent-exposed and interior positions, indicating increased conformational stability rather than steric restriction from the bound cofactor alone.

In both the apo- and the holo-structures, the interior side chains stack into four layers, beginning at the edge of the porphyrin-binding site and extending to the end of the bundle (FIGS. 5D-5F). In the absence of cofactor to constrain and stabilize the tightly packed conformation of the holo-protein, the layers closest to the binding site explore more conformations, accessing rotamers not seen in holo-PS1 (FIG. 5E). By contrast, the packing of the more distal layers is identical in the apo- and holo-structures (FIG. 5F). Thus, the third- and fourth-shell layers, located up to 20 Å away from the binding site, are precisely pre-organized to stabilize the conformation of the first-shell side chains when PS1 enfolds its cofactor. This finding is consistent with numerous studies on natural proteins¹³⁻¹⁶, which show that variation of residues involved in core packing distant from an active site can have profound influences on binding and catalysis.

The vast improvement in conformational specificity between PS1 and earlier designs illuminates the importance of considering hydrophobic core packing and the construction of ligand-binding sites as a joint optimization problem during computational design. Our previous studies indicate that the use of rigid backbones optimized for ligand-protein interactions alone are insufficient for conformational uniqueness without explicitly considering and designing a backbone that can also accommodate a well-defined apolar core. Similarly, attempts to radically change specificity of natural proteins by varying their binding sites, while treating the surrounding protein matrix as a rigid unit of fixed sequence, has required subsequent experimental optimization via extensive rounds of random mutagenesis and selection^{18,19,39}. The reliance on experimental methods such as directed evolution and genetic selections, while currently useful in many practical applications¹⁹, speaks to our incomplete understanding of protein structure and function, and the need to test and refine this knowledge through design. It is noteworthy that the first sequence designed and tested via our approach succeeded without need for experimental screening. Furthermore, another high-scoring protein design also bound the cofactor, suggesting a possible generality of the method within the helical bundle protein family. These studies bring chemists closer to the ultimate goal of the computational design of fully functional proteins with properties unprecedented in nature.

PS1 Design Process.

Full methods and scripts regarding the design of PS1 can be found in Example 2. Briefly, the entire core of the D2-symmetrical parameterized backbone of SCRPZ-2 was redesigned to bind (CF₃)₄PZn via a customized Rosetta script for flexible backbone sequence design. The flexible backbone design protocol was as follows: Distance and angle constraints between His and Zn were loaded, the model was repacked without mutations, the backbone was relaxed via Rosetta Backrub, three trials of a Monte Carlo flexible backbone design sub-protocol (see Example 2) were performed, and models with native protein-like packing (i.e., a Rosetta PackStat score≥0.58) were output. 170 designs were output from 500 runs through the protocol (FIG. 6). We analyzed these 170 models for packing, radius of gyration, energy, and rotamer state probability within Matlab to select PS1 for expression. The design of PS2 proceeded in the same fashion.

Protein Expression, Purification, and Biophysical Characterization.

Details regarding protein expression, purification, and biophysical characterization can be found in the supplement. Briefly, genes for the proteins were ordered from GenScript, cloned into a pET-11a plasmid, and purified via a Ni column, followed by His-tag cleavage by TEV protease. The protein sequence of expressed, purified PS1 after His-tag cleavage is: SEFEKLRQTGDELVQAFQRLREIFDKGDDDSLEQVLEEIEELIQKHRQLFDNRQEAADTE AAKQGDQWVQLFQRFREAIDKGDKDSLEQLLEELEQALQKIRELAEKKN (SEQ ID NO:2). The sequence for PS2 can be found in Example 2.

Porphyrin Binding to Apo-Protein.

A 2-fold excess of the cofactor (CF₃)₄PZn was added from a 4 mM DMSO stock solution to a 50 mM NaPi, 100 mM NaCl, pH 7.5 buffer with apo-protein (Note that final DMSO concentrations were kept <1%.). Buffer solution of apo-PS1 protein was heated for 5 minutes at 50° C., (CF₃)₄PZn was then added from the DMSO stock solution, the resultant mixture was vortexed for 5 seconds, and placed back in the heat block at 50° C. for 15 minutes, with vortexing every 3 minutes. The protein/cofactor solution was then spun at 14000×g in a Amicon Ultra-0.5 mL centrifuge filter for 10 min, three times, replacing the buffer to 0.5 mL after each 10 min spin. Finally, the protein solution was spun for 4 min at 12000×g in an Amicon ultrafree-MC GV filter (UFC30GV0S). The holo-PS1 sample was then used for spectroscopic experiments immediately afterward, and diluted to an appropriate concentration if necessary. Binding of (CF₃)₄PFe was carried out in the same fashion, with the exception that the porphyrin was first dissolved in a stock of DMSO/CHCl₃.

Nuclear Magnetic Resonance Spectroscopy.

NMR spectra were recorded at 298 K on a 900 MHz Bruker Avance II spectrometer equipped with cryogenic probe for the holo-protein or on a Bruker 600 MHz spectrometer equipped with cryogenic probe for the apo-protein.

Sequence specific backbone (¹H^{N}, ¹⁵N, ¹³C^{α}, ¹³CO) and ¹³C^{β} resonance assignments were obtained by using 3D HNCACB/CBCA(CO)NH and 3D HNCO/CO(CA)NH along with the program AUTOASSIGN.^{41 1}H^{α} and ¹H^{β} assignments were extended by 3D HAHB(CO)NH experiment and more peripheral side chain chemical shifts were assigned with aliphatic 3D CCH-TOCSY (mixing time: 75 ms) and simultaneous 3D ¹⁵N/¹³C^{aliphatic}/¹³C^{aromatic}-resolved [¹H,¹H]-NOESY (mixing time: 120 ms). Overall assignments were obtained for 98.1% and 95.9% of the backbone (excluding the N-terminal NH₃⁺) and ¹³CO, and for 97% and 94.6% of the side chain chemical shifts (excluding Lys NH₃⁺, Arg NH₂, OH, side chain ¹³CO and aromatic ¹³C^{γ}) for the holo- and apo-proteins, respectively. All spectra were processed and analyzed with the programs NMRPIPE and XEASY, respectively^{42,43}. ¹H□¹H upper distance limit constraints for structure calculations were extracted from NOESY. In addition, backbone dihedral angle constraints were derived from chemical shifts using the program TALOS for residues located in well-defined secondary structure elements⁴⁴. 2D constant-time [¹³C,¹H]-HSQC spectra were recorded as was described for the 5% fractionally ¹³C-labeled samples to obtain stereo-specific assignments for isopropyl groups of Val and Leu⁴⁵. The ¹D_{NH} residual dipolar couplings (RDCs) were measured with 2D ¹H-¹⁵N IPAP-HSQC in samples aligned using Pf1 phage (ASLA biotech). The program CYANA was used to assign long-range NOEs and calculate the structure^{46,47}. Backbone ¹D_{NH} RDCs were used as orientational constraints for the later stages of refinement with XPLOR-NIH⁴⁸. The final set of structures was further refined by restrained molecular dynamics in explicit water⁴⁸. NMR structure quality was assessed with the Protein Structure Validation Software Suite (PSVS)⁴⁹ (Table S4).

Hydrogen-Deuterium Exchange Measurements.

For the measurements of H/D exchange rates, a series of 2D ¹⁵N HSQC spectra were obtained on a 900 MHz Bruker Avance II spectrometer. The first spectra were recorded 9 minutes after the dilution of 100 μl of a high concentration sample in H₂O (2 mM for apo and 1.2 mM for holo) into 200 μl D₂O buffer. 15-min HSQC spectra were recorded successively in the first 12 hours, a 15-min spectrum in every hour in the second 12 hours, a 15-min spectrum in every two hours in the third 12 hours, and so on. The last points were 2730.6 and 4903.5 min for apo and holo, respectively. For the H/D exchange rate analysis, the peak height of each isolated peak was extracted by nmrDraw and fitted to one-phase exponential decay.

Coordinates and data files have been deposited to the Protein Data Bank with accession codes STGW (apo-PS1) and STGY(holo-PS1) and to the BMRB (chemical shifts) with codes 30185 (apo-PS1) and 30186 (holo-PS1).

References cited in Example 1. 1. Roy, S. et al. A protein designed by binary patterning of polar and nonpolar amino acids displays native-like properties. J. Am. Chem. Soc. 119, 5302-5306 (1997). 2. Kuhlman, B. et al. Design of a novel globular protein fold with atomic-level accuracy. Science 302, 1364-1368 (2003). 3. Nanda, V. & Koder, R. L. Designing artificial enzymes by intuition and computation. Nat. Chem. 2, 15-24 (2010). 4. Peacock, A. F. A. Incorporating metals into de novo proteins. Curr. Opin. Chem. Biol. 17, 934-939 (2013). 5. Huang, P.-S. et al. High thermodynamic stability of parametrically designed helical bundles. Science 346, 481 (2014). 6. Thomson, A. R. et al. Computational design of water-soluble α-helical barrels. Science 346, 485 (2014). 7. Woolfson, D. N. et al. De novo protein design: How do we expand into the universe of possible protein structures? Curr. Opin. Struct. Biol. 33, 16-26 (2015). 8. Mocny, C. S. & Pecoraro, V. L. De novo protein design as a methodology for synthetic bioinorganic chemistry. Acc. Chem. Res. 48, 2388-2396 (2015). 9. Ulas, G., Lemmin, T., Wu, Y., Gassner, G. T. & DeGrado, W. F. Designed metalloprotein stabilizes a semiquinone radical. Nat. Chem. 8, 354-359 (2016). 10. Olson, T. L. et al. Design of dinuclear manganese cofactors for bacterial reaction centers. Biochim. Biophys. Acta: Bioenergetics 1857, 539-547 (2016). 11. Huang, P.-S., Boyken, S. E. & Baker, D. The coming of age of de novo protein design. Nature 537, 320-327 (2016). 12. Brunette, T. J. et al. Exploring the repeat protein universe through computational protein design. Nature 528, 580-584 (2015). 13. Bollen, Y. J. M., Westphal, A. H., Lindhoud, S., van Berkel, W. J. H. & van Mierlo, C. P. M. Distant residues mediate picomolar binding affinity of a protein cofactor. Nat. Comm. 3, 1010 (2012). 14. Sela-Culang, I., Kunik, V. & Ofran, Y. The structural basis of antibody-antigen recognition. Front. Immunol. 4 (2013). 15. van den Bedem, H., Bhabha, G., Yang, K., Wright, P. E. & Fraser, J. S. Automated identification of functional dynamic contact networks from X-ray crystallography. Nat. Methods 10, 896-902 (2013). 16. Koulechova, D. A., Tripp, K. W., Horner, G. & Marqusee, S. When the scaffold cannot be ignored: The role of the hydrophobic core in ligand binding and specificity. J. Mol. Biol. 427, 3316-3326 (2015). 17. Reedy, C. J. & Gibney, B. R. Heme protein assemblies. Chem. Rev. 104, 617-650 (2004). 18. Tinberg, C. E. et al. Computational design of ligand-binding proteins with high affinity and selectivity. Nature 501, 212-216 (2013). 19. Prier, C. K. & Arnold, F. H. Chemomimetic biocatalysis: Exploiting the synthetic potential of cofactor-dependent enzymes to create new catalysts. J. Am. Chem. Soc. 137, 13992-14006 (2015). 20. Farid, T. A. et al. Elementary tetrahelical protein design for diverse oxidoreductase functions. Nat. Chem. Biol. 9, 826-833 (2013). 21. Skalicky, J. J. et al. Solution structure of a designed four-α-helix bundle maquette scaffold. J. Am. Chem. Soc. 121, 4941-4951 (1999). 22. Huang, S. S., Koder, R. L., Lewis, M., Wand, A. J. & Dutton, P. L. The HP-1 maquette: From an apoprotein structure to a structured hemoprotein designed to promote redox-coupled proton exchange. Proc. Natl. Acad. Sci. USA 101, 5536-5541 (2004). 23. Lombardi, A., Nastri, F. & Pavone, V. Peptide-based heme-protein models. Chem. Rev. 101, 3165-3190 (2001). 24. Huang, S. S., Gibney, B. R., Stayrook, S. E., Leslie Dutton, P. & Lewis, M. X-ray structure of a maquette scaffold. J. Mol. Biol. 326, 1219-1225 (2003). 25. Gibney, B. R., Rabanal, F., Skalicky, J. J., Wand, A. J. & Dutton, P. L. Iterative protein redesign. J. Am. Chem. Soc. 121, 4952-4960 (1999). 26. Bender, G. M. et al. De novo design of a single-chain diphenylporphyrin metalloprotein. J. Am. Chem. Soc. 129, 10732-10740 (2007). 27. Fry, H. C., Lehmann, A., Saven, J. G., DeGrado, W. F. & Therien, M. J. Computational design and elaboration of a de novo heterotetrameric alpha-helical protein that selectively binds an emissive abiological (porphinato)zinc chromophore. J. Am. Chem. Soc. 132, 3997-4005 (2010). 28. Fry, H. C. et al. Computational de novo design and characterization of a protein that selectively binds a highly hyperpolarizable abiological chromophore. J. Am. Chem. Soc. 135, 13914-13926 (2013). 29. Solomon, L. A., Kodali, G., Moser, C. C. & Dutton, P. L. Engineering the assembly of heme cofactors in man-made proteins. J. Am. Chem. Soc. 136, 3192-3199 (2014). 30. Ghirlanda, G. et al. De novo design of a D₂-symmetrical protein that reproduces the diheme four-helix bundle in cytochrome bc₁. J. Am. Chem. Soc. 126, 8141-8147 (2004). 31. North, B., Summa, C. M., Ghirlanda, G. & DeGrado, W. F. Dₙ-symmetrical tertiary templates for the design of tubular proteins. J. Mol. Biol. 311, 1081-1090 (2001). 32. Lahr, S. J. et al. Analysis and design of turns in α-helical hairpins. J. Mol. Biol. 346, 1441-1454 (2005). 33. Goll, J. G., Moore, K. T., Ghosh, A. & Therien, M. J. Synthesis, structure, electronic spectroscopy, photophysics, electrochemistry, and x-ray photoelectron spectroscopy of highly-electron-deficient [5,10,15,20-tetrakis(perfluoroalkyl)porphinato]zinc(II) complexes and their free base derivatives. J. Am. Chem. Soc. 118, 8344-8354 (1996). 34. Lubitz, W., Lendzian, F. & Bittl, R. Radicals, radical pairs and triplet states in photosynthesis. Acc. Chem. Res. 35, 313-320 (2002). 35. Kaufmann, K. W., Lemmon, G. H., DeLuca, S. L., Sheehan, J. H. & Meiler, J. Practically useful: What the Rosetta protein modeling suite can do for you. Biochemistry 49, 2987-2998 (2010). 36. Moore, K. T., Horvath, I. T. & Therien, M. J. Mechanistic studies of (porphinato)iron-catalyzed isobutane oxidation. Comparative studies of three classes of electron-deficient porphyrin catalysts. Inorg. Chem. 39, 3125-3139 (2000). 37. Gentemann, S. et al. Variations and temperature dependence of the excited state properties of conformationally and electronically perturbed zinc and free base porphyrins. J. Phys. Chem. B 101, 1247-1254 (1997). 38. Bradley, P., Misura, K. M. S. & Baker, D. Toward high-resolution de novo structure prediction for small proteins. Science 309, 1868 (2005). 39. Tinberg, C. E. & Khare, S. D. in Computational Design of Ligand Binding Proteins (ed Barry L. Stoddard) 155-171 (Springer New York, 2016). 40. Choma, C. T. et al. Design of a heme-binding four-helix bundle. J. Am. Chem. Soc. 116, 856-865 (1994). 41. Zimmerman, D. E. et al. Automated analysis of protein NMR assignments using methods from artificial intelligence. J. Mol. Biol. 269, 592-610 (1997). 42. Delaglio, F. et al. NMRPipe: a multidimensional spectral processing system based on UNIX pipes. J. Biomol. NMR 6, 277-293 (1995). 43. Bartels, C., Xia, T.-h., Billeter, M., Guntert, P. & Wiithrich, K. The program XEASY for computer-supported NMR spectral analysis of biological macromolecules. J. Biomol. NMR 6, 1-10 (1995). 44. Cornilescu, G., Delaglio, F. & Bax, A. Protein backbone angle restraints from searching a database for chemical shift and sequence homology. J. Biomol. NMR 13, 289-302 (1999). 45. Neri, D., Szyperski, T., Otting, G., Senn, H. & Wuethrich, K. Stereospecific nuclear magnetic resonance assignments of the methyl groups of valine and leucine in the DNA-binding domain of the 434 repressor by biosynthetically directed fractional carbon-13 labeling. Biochemistry 28, 7510-7516 (1989). 46. Guntert, P., Mumenthaler, C. & Wüthrich, K. Torsion angle dynamics for NMR structure calculation with the new program DYANA. J. Mol. Biol. 273, 283-298 (1997). 47. Herrmann, T., Guntert, P. & Wiithrich, K. Protein NMR structure determination with automated NOE assignment using the new software CANDID and the torsion angle dynamics algorithm DYANA. J. Mol. Biol. 319, 209-227 (2002). 48. Schwieters, C. D., Kuszewski, J. J., Tjandra, N. & Marius Clore, G. The Xplor-NIH NMR molecular structure determination package. J. Magn. Reson. 160, 65-73 (2003). 49. Bagaria, A., Jaravine, V., Huang, Y. J., Montelione, G. T. & Guntert, P. Protein structure validation by generalized linear model root-mean-square deviation prediction. Protein Sci. 21, 229-238 (2012).

### Example 2—Computational and Synthesis Methods

PS1 Design Process.

The design of PS1 began with a D₂-symmetrical parameterized backbone of a 4-helix bundle (Tables S1 and S2)¹. We have previously used this backbone parameterization to create a D₂-symmetrical diheme-binding tetrameric 4-helix bundle, PA_{TET}, which was composed of 4 copies of a 25 residue helix containing the requisite metal-coordinating His and second shell H-bonding Thr residues placed at d and b positions in a heptad repeat, respectively². This tetramer bound two hemes with a bis-His ligation in a D₂-symmetrical bundle. Asymmetry of the sequence was later introduced in a single chain diporphyrin-binding design, PA_{SC} (FIG. 1B), where loops were selected to connect the helices via a structural bioinformatics approach^{3,4}. The attachment of these loops cemented the Crick parameters of the helical backbone, which was later employed in another single-chain protein design, SCRPZ-2, that bound an extended cofactor throughout the interior of the bundle (FIG. 1B)⁵. The design of PS1 utilized the His and Thr positioning of one porphyrin-binding region from these previous designs, with the remainder of the protein then designated as a cofactor-free folded core. Because SCRPZ-2 was soluble and expressed well in E. coli, we elected to retain its exterior-facing amino acids and loops within the PS1 design, while computationally designing the entire core (binding region and folded core simultaneously). In doing so, we also isolate effects on cofactor binding due solely to the creation of a folded core that uniquely predisposes the binding region for cofactor association, which is simultaneously optimized for sequence and side chain packing along with the binding region of the (CF₃)₄PZn porphyrin. A flexible backbone sequence design protocol was developed (see below) to fine-tune the parameterized backbone to (CF₃)₄PZn and to achieve optimal side chain packing for creation of the folded core and positioning of (CF₃)₄PZn within the binding region.

Flexible Backbone Sequence Design.

We wrote a RosettaScript for flexible backbone sequence design, implemented in Rosetta 3.5, that proceeds through a cycle of backbone/sidechain relaxations and fixed backbone design, with a filtering step based on core packing (RosettaScript provided below). Details of the process are provided in the subsections herein.

Amino Acids Allowed to Vary in the Design.

Because (CF₃)₄PZn could potentially act as a photo-oxidant, we disallowed any potentially oxidizable amino acids in the sequence (e.g., Tyr, Cys, Met, Trp, His) other than the single His and Trp residues described below. The initial residue identities of the bundle were chosen from a previous computationally designed 4-helix bundle SCRPZ-2⁵, with a few changes, e.g., surface-exposed Tyr residues of the SCRPZ-2 sequence were constrained to be polar or charged during the computational sequence design in Rosetta. The entire core (40 residues in total) of SCRPZ-2 was allowed to vary during the design process, except for His46 and Thr9, which are keystone interactions dedicated to Zn coordination of the porphyrin used in previous designs (see FIG. 2). (63% of the SCRPZ-2 sequence consists of exterior residues and loops, and these were held fixed during the design of PS1.) Ultimately, of the 40 residues that could vary (out of 108), 28 residues were changed and 12 were retained from the SCRPZ-2 sequence, such that 70% of the core was computationally mutated to establish a preferred orientation of the porphyrin cofactor, as well as an interdigitated folded core. This percentage of retained residues can be rationalized based on the expected results of choosing large space-filling amino acids (Phe, Leu, Ile, Val) at random, such that a residue that is Leu in the sequence has a 25% chance of retaining its identity as Leu. Table S3 and FIG. 6B, as well as the residue file (resfile.txt) below, show precisely which residues were allowed to vary during the design process. Below and in the main text, we use residue numbering that is consistent with the expressed holo-protein, which contains an N-terminal Ser residue not present in the design, a remnant from a TEV protease cleavage site (see below).

Selection of Residue 68 as Trp.

A motivation for this work is to position aromatic side chains in precise position relative to a photo-excitable cofactor to initiate proton-coupled electron transfer. We asked whether a Trp residue could be held in precise juxtaposition relative to the (CF₃)₄PZn cofactor, as a prelude to future studies in which “proton wires” are introduced to facilitate proton transfer concomitant with electron transfer from Trp to the photoexcited state of (CF₃)₄PZn. A Trp residue in the protein interior also serves as an absorption handle, as well as a fluorescent indicator of hydrophobic packing.

To select the sequence position of the single Trp residue, we used the Rosetta Backrub program^{6,7} to create an ensemble of backbones that were relaxed around the (CF₃)₄PZn cofactor, after the cofactor was docked in the porphyrin binding region of the SCRPZ-2 model, with an orientation described by CF₃ groups pointing down the long axis of the bundle. No sequence design was performed to generate this backbone ensemble. Next, we performed fixed backbone sequence design on each member of the backbone ensemble, allowing Trp at all core residues, to determine a probable location of Trp within the protein interior, based on the frequency of occurrence within the designed sequences. Based on this information, we constrained residue 68 to be Trp during the flexible backbone design process below.

Flexible Backbone Design Protocol.

Flexible backbone design utilized angle and distance constraints between the Zn and His to restrict the design space to those consistent with the DFT-optimized imidazole-Zn distance of 2.0 Å. We used an energy term (hack_aro=1) that models quadrupolar interactions between aromatic side chains in every stage of the flexible backbone design protocol. We also employed an energy term (rg=2) that penalizes bundles with a large radius of gyration (rg). We noticed a propensity within Rosetta to output bundles that received good packing scores (via Packstat or Rosetta Holes) but displayed helices separated by large distances (large rg). The packing algorithms could not differentiate between interior or exterior when the helix-helix interfaces were very wide, and often inappropriately gave good packing scores when the designed bundle was qualitatively poorly packed. The inclusion of the rg term, as well as employing Rosetta Backrub, ameliorated this issue.

The flexible backbone design protocol was as follows: Distance and angle constraints between His and Zn were loaded, the model was repacked without mutations, the backbone was relaxed via Rosetta Backrub, three trials of a Monte Carlo flexible backbone design sub-protocol (see below) were performed, and models with native protein-like packing (i.e., a Rosetta PackStat score≥0.58) were output. The PackStat score was calculated 3 times per trial to account for its stochastic behavior. 170 designs were output from 500 runs through the protocol (Fig. S1). We analyzed these 170 models for packing, rg, energy, and rotamer state probability within Matlab to select PS1 for expression.

Flexible Backbone Design Sub-Protocol.

The flexible backbone design sub-protocol consists of 3 Monte Carlo trials of (i) fixed backbone design with soft weights (decreased vdW interactions, i.e., soft_rep_design weights within Rosetta), (ii) sidechain minimization via MinMover, (iii) fixed backbone design with Score13 weights, where the electrostatic term (fa_pair) is replaced by hack_elec (hack_elec=0.55), and the addition of extra rotamer sampling around χ₁ (ex1, level 3, i.e., sampled between 2 std of the mean chi angle value for each rotamer) and χ₂ (ex2, level 3) sidechain dihedrals, (iv) backbone minimization via MinMover, (v) repetition of step iii (due to propensities of Rosetta to design a particular sequence to a particular backbone). At the end of step (v), the model is filtered for native structure-like packing via PackStat (If 1 of 3 trials of PackStat score is >0.58, the model passes the filter.). In all energy functions for flexible backbone design, hack_aro is set to 1 and rg is set to 2. The final, designed sequence (PS1) selected for protein expression was the following 108 amino acids:

(SEQ ID NO: 1)
EFEKLRQTGDELVQAFQRLREIFDKGDDDSLEQVLEEIEELIQKHRQLEDN
RQEAADTEAAKQGDQWVQLFQRFREAIDKGDKDSLEQLLEELEQALQKIRE
LAEKKN

Ab initio folding.

Rosetta ab initio folding⁸ was performed on the PS1 sequence in Rosetta 3.5. Ca RMSD of the folded core was scored against residues 14-23, 32-42, 69-79, and 87-97 of the design model. Ca RMSD of the binding region was scored against residues 5-13, 43-50, 61-68, and 98-105 of the design model.

Porphyrin Binding Titration to Determine K_{D}.

2 μM of (CF₃)₄PZn was solubilized in a 1 mL solution of 50 mM NaPi, 100 mM NaCl, pH 7.5 buffer by inclusion of 1% w/v octyl-b-D-glucopyranoside. 2 μL of a 102 uM stock of apo-PS1 (0.2 μM aliquots) was titrated into the 1 mL solution containing the porphyrin, and an electronic absorption spectrum was measured until >2.5 equivalents of protein were added. Absorbance changes at 423 nm, due to His-Zn coordination-induced spectral shifts of the porphyrin, were fit to a single-site, protein-ligand binding model.

Analytical Ultracentrifugation (AUC).

The oligomeric state of apo- and holo-PS1 were determined by analytical equilibrium sedimentation performed at 25° C. using a Beckman XL-I analytical ultracentrifuge. Ultracentrifugation was conducted at speeds of 25K, 30K, 35K, 40K and 45K r.p.m., and the radial gradient profiles were obtained by absorbance at 280 nm. A 200 μM solution of the apo- and a 100 μM solution of the holo-protein were prepared in 50 mM NaPi pH 7.5, 100 mM NaCl (apo) and 20 mM NaPi pH 7.5, 125 mM NaCl (holo). Data were globally fit to a single-species model of equilibrium sedimentation by a nonlinear least-squares method using IGOR Pro (Wavemetrics).

Size Exclusion Chromatography.

Gel filtration profiles were obtained using a Superdex 75 5/150 column on an FPLC system (GE Healthcare AKTA). To evaluate the oligomeric state, 20 μL of 100 μM apo-PS1 or 37 μM holo-PS1 was injected onto the column and eluted with a 50 mM phosphate, 150 mM NaCl, pH 7.0 buffer mobile phase at a flow rate of 0.4 mL/min. The approximate molecular weight (MWₐₚₚ) was calculated from a standard curve obtained with the GE LMW standard protein kit. From this curve, MWₐₚₚ of the apo is 19.5 kD and that of holo is 17.9 kD. These 13 kD proteins elute at higher MWₐₚₚ due to their large negative surface charge (q=−12). For apo-PS1, a small dimer peak elutes at MWₐₚₚ of 44.1 kD, and a smaller tetramer (or pentamer) peak at 103.2 kD.

Circular dichroism (CD).

CD spectra were collected on a Jasco J-810 CD spectrometer in a 0.1 cm path length quartz cuvette, using temperature/wavelength mode. Spectra were collected from 20 to 95° C. with an interval of 5° C. and an increase rate of 1° C./minute, over a wavelength range from 215 to 250 nm. Apo- and holo-PS1 were prepared at 10 μM and 6.6 μM, respectively, in 50 mM NaPi pH 7.5, 100 mM NaCl buffer. Temperature melts of apo-PS1 were also performed at varying concentrations of Guanidine HCl denaturant (0M, 1M, 2M, 3M, 4M, 5M, 5.85, 7M).

Steady-State Electronic Absorption and Emission Spectroscopy.

Electronic absorption spectra were collected using a Shimadzu UV-1700 UV-Vis spectrophotometer or Cary 5000 spectrophotometer. Steady-state emission spectra were obtained on FLS920P spectrophotometer (Edinburgh Instruments Ltd. Livingston, UK) in 1 cm quartz optical cells. The steady-state emission spectra were corrected using the correction factor generated by the manufacturer.

Pump-Probe Transient Absorption Spectroscopy.

Ultrafast transient absorption spectra were obtained using standard pump-probe methods⁹ with a time resolution of approximately 200 fs. Elevated temperature experiments were performed in a custom-made temperature block of anodized aluminum, the temperature of which was controlled by heating rods and monitored by a pair of thermocouples wired to a PID through a solid-state relay. Following pump-probe transient absorption experiments, electronic absorption spectra verified that the samples were robust.

Cofactor (e.g., Ligand) Geometry Optimization.

The geometry of (CF₃)₄PZn was optimized via density functional theory using the B3-LYP functional and 6-31G* basis set implemented in Gaussian03. The starting geometry was obtained from the crystal structure of related meso-heptafluoropropyl(porphinato)Zn(II), with the fluoropropyl groups truncated to fluoromethyl¹⁰. Meso-heptafluoropropyl(porphinato)Zn(II) co-crystallized with an axially ligating pyridine; imidazole was computationally substituted for pyridine for the geometry optimization of (CF₃)₄PZn.

Visualization of Protein Structures and Image Rendering.

Protein models were visualized and rendered in the PyMol visualization program¹¹.

Protein expression and purification. The gene coding for the protein sequence of PS1 was ordered from GenScript, which was cloned into the IPTG-inducible pET-11a plasmid (cloning site NdeI-BamHI). The sequence also coded for an N-terminal 6×His-tag followed by a TEV protease cleavage sequence, followed finally by the designed sequence. The cloned gene sequence is:

(SEQ ID NO: 3)
CATATGCATCACCATCACCATCACGAAAACCTGTATTTTCAGAGCGAATTC
GAAAAACTGCGTCAAACCGGCGACGAACTGGTGCAGGCATTTCAACGTCTG
CGCGAAATTTTCGATAAAGGTGATGACGATAGTCTGGAACAGGTTCTGGAA
GAAATTGAAGAACTGATCCAGAAACATCGTCAACTGTTTGACAATCGCCAG
GAAGCGGCCGATACGGAAGCAGCTAAACAGGGCGACCAATGGGTCCAGCTG
TTTCAACGTTTCCGCGAAGCCATTGATAAAGGTGACAAAGATAGCCTGGAA
CAGCTGCTGGAAGAACTGGAACAGGCGCTGCAAAAAATCCGCGAACTGGCC
GAAAAGAAAAACTAAGGATCC

The expressed protein sequence was ultimately:

(SEQ ID NO: 4)
MHHHHHHENLYFQ/SEFEKLRQTGDELVQAFQRLREIFDKGDDDSLEQVLE
EIEELIQKHRQLFDNRQEAADTEAAKQGDQWVQLFQRFREAIDKGDKDSLE
QLLEELEQALQKIRELAEKKN

where the “/” defines the cleavage site of TEV protease. The plasmids were transfected into E. coli BL21(DE3) cells, which were grown in LB/ampicillin media (or, for NMR samples, M9 minimal media with isotope-labeled ammonia and glucose from Cambridge Isotopes) until OD @ 600 nm=0.6. The cells were then induced with IPTG and allowed to grow for 4 more hours. Cells were then centrifuged and frozen. The frozen cell pellets were lysed in a French press in the Duke University Biology Department. The expressed, His-tagged PS1 protein was purified via a Ni NTA column (Invitrogen) and confirmed by gel electrophoresis. The buffer was exchanged to the Sigma-recommended TEV protease buffer (5 mM DTT, 50 mM Tris, 0.5 mM EDTA, pH 8.0), and the PS1/TEV solution (His-tagged TEV protease was ordered from Sigma.) was allowed to rock for 1 day at room temperature. The resulting His-tag-free PS1 protein was collected from the flow-through of a Ni NTA column and concentrated in a stock of 50 mM NaPi, 100 mM NaCl, pH 7.5 buffer, with an approximate yield of 40 mg/L. PS2 was expressed and purified in the same manner.

Design of PS2.

To explore the need for a second shell hydrogen bond to the Trp indole of W68, we designed a second sequence, PS2. Computational evaluation of positions where a second-shell polar residue could be introduced showed that a Ser at position 94 could form the desired hydrogen bond. This residue is Leu in PS1, so introducing a small Ser at this position led to a local defect in the packing if this change were made directly into PS1. Thus, the entire core was redesigned using the original procedure, but this time requiring Ser and Trp at positions 94 and 68, respectively. The core of PS2 shares only 55 percent identity with PS1, as shown in the aligned sequences below. (The solvent-exposed amino acids are identical between PS1 and PS2, as per the design method, which only explicitly considers the protein core.)

Core residues of PS1:
(SEQ ID NO: 5)
LGLVAFLIFGLVLILIHLFAAGWVFFAILLLLALILA
Core residues of PS2:
(SEQ ID NO: 6)
LGIILLLAIGLILLAFHLFFAGWLFIAILLFSGIILA

PS2 was expressed with the same His-tag as PS1, and cleaved and purified using the same methods. Binding of (CF₃)₄PZn to PS2 was carried out using the same method as for PS1. We found that PS2 bound (CF₃)₄PZn in a homogenous environment, indicated by the narrow electronic absorption bands of the porphyrin in PS2, nearly indistinguishable from that in PS1 (FIG. 10). PS2 will be structurally characterized in future studies in which we will examine the role of second and third-shell hydrogen bonds on the photophyiscal properties of holo-PS proteins. The expressed, purified, His-tag cleaved sequence of PS2 was:

(SEQ ID NO: 7)
SEFEKLRQTGDEIIQLLQRLREAIDKGDDDSLEQILEELEEAFQKHRQLFE
NRQEAADTEFAKQGDQWLQLFQRIREAIDKGDKDSLEQLFEESEQGIQKIR
ELAEKKN

Cofactor Synthesis.

The cofactor [5,10,15,20-tetrakis(trifluoromethyl)porphinato]zinc(II), abbreviated as (CF₃)₄PZn in the main text, was synthesized as previously reported″, and was confirmed by NMR and electronic absorption spectra. Likewise for (CF₃)₄PFe.

Clustering of Apo-PS1 NMR Models.

We implemented a greedy clustering algorithm in Matlab to form clusters within the family of structures of apo-PS1 (Extended Data FIG. 7). A pairwise RMSD matrix of each apo-PS1 model was scored against residues 61-67 and 99-105. These residues, which lie on opposite helices, show the largest conformational variation within the apo-PS1 models. The clustering algorithm defines the centroid as the column of the RMSD matrix containing the largest number of RMSD values below a threshold of 1 Å. Components of this column below this threshold have their corresponding rows and columns removed from the RMSD matrix, and the clustering algorithm repeats again on this truncated RMSD matrix. Of the 20 NMR models, two clusters were found with >4 members each. The cluster defining the closed conformation contained 13 members, and that of the open conformation contained 5 members.

Molecular Dynamics Simulations.

The lowest-energy NMR structure of apo-PS1, which is the centroid of the closed conformation, was used as the starting conformation for the molecular dynamics simulation. The structure was solvated in a 17 Å padding water box, neutralized by the addition of 12 Na⁺ counter ions. The AMBER force field 14SB was used for the parameterization of the protein. TIP3P water parameterization was used to describe the water molecules¹².

The molecular dynamics simulation was carried out using ACEMD¹³. The system was minimized for 2000 steps, followed by equilibration using the NPT ensemble for 10 ns at 1 atm using a time-step of 2 fs. We also used rigid bonds and a cutoff of 9 Å using PME for long-range electrostatics. Following the relaxation phase, the protein was allowed to move freely and simulated under the NVT ensemble using ACEMD's NVT ensemble with a Langevin thermostat. To achieve a time-step of 4 ps, we used damping at 0.1 ps-1 and a hydrogen mass repartitioning scheme. The simulation was carried out to 1 μs at 298 K.

SOCKET Server for assessment of knobs-into-holes packing. PDB files of the PS1 design model, holo-PS1 centroid, and apo-PS1 open/closed centroids were individually uploaded to and analyzed by the SOCKET server¹⁴ for knobs-into-holes side chain packing (see Section 4). A helical residue was defined as a knob if its side chain was within 8 Å of 4 other side chains from residues on an adjacent helix (a hole). Output from the SOCKET server for each of these PDB files is displayed below showing the residues of each knob and hole. Note that the residue number of the PS1 design model is off register by 1 amino acid from the structural sequences, due to the presence of the N-terminal Ser residue from TEV cleavage of the expressed proteins.

### Example 3—enFold Proteins can Bind Endogenous Ligands

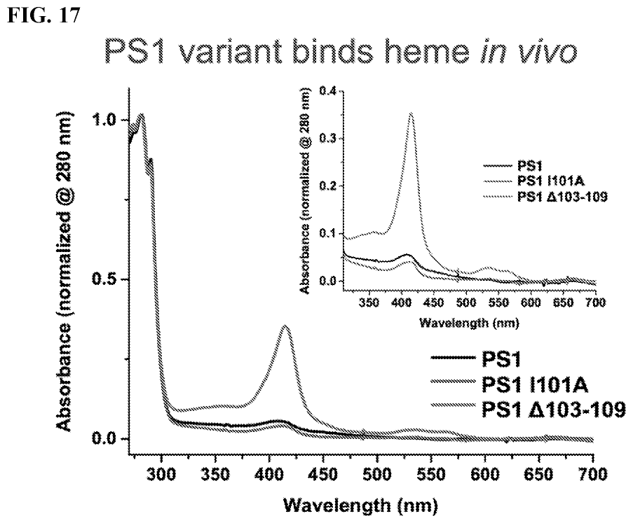

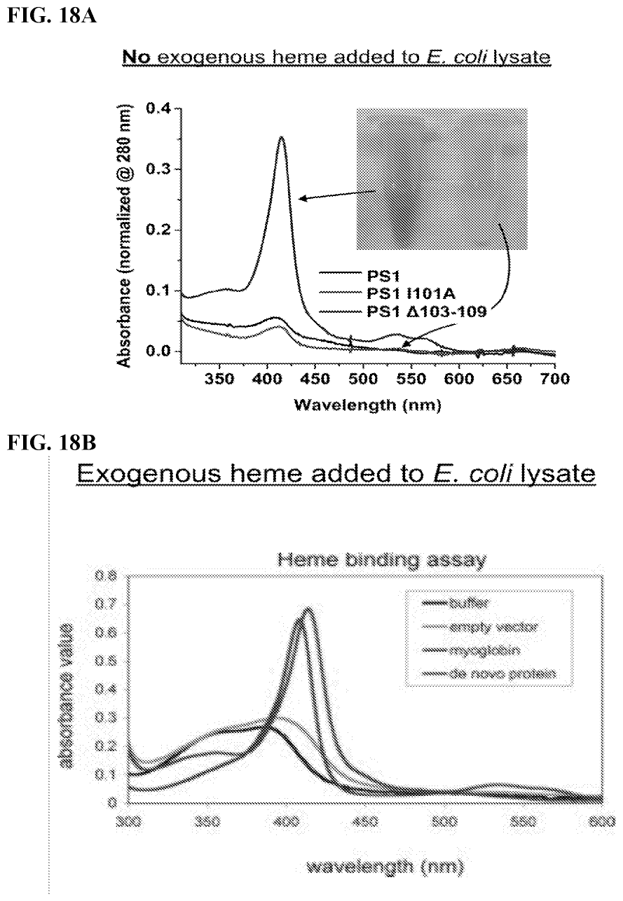

The computational method described here is capable of producing proteins that noncovalently bind ligands in vivo. We have observed loading of endogenous heme in a PS1 variant, where 7 terminal residues near the binding ligand site were deleted to allow incorporation of a heme ligand with its bulky, charged proprionate functional groups (FIG. 16). The design methodology produces proteins which possess unique structure in the apo-form to avoid aggregation even at high concentration, which may occur during cellular expression. These apo-proteins remain competent to bind an endogenous ligand, for example heme (FIG. 17 and FIGS. 18A-18B). These proteins are the first de novo designed proteins to our knowledge that noncovalently bind heme in vivo.

### Data Tables

TABLE S1
Best-fit Crick parameters* at various stages
of PS1 design (no symmetry constraint)

| Best-fit Crick | Starting | PS1 design | Holo-PS1 centroid |
|---|---|---|---|
| parameters | backbone | backbone | backbone |
| R₀ (Å) | 8.020 | 7.747 | 7.902 |
| R₁(Å) | 2.228 | 2.254 | 2.250 |
| ω₀ (°/res) | −2.593 | −2.939 | −3.114 |
| ω₁ (°/res) | 102.771 | 102.767 | 102.325 |
| α (°) | −13.153 | −15.202 | −16.635 |
| φ₁ for chain A (°) | −71.718 | −72.480 | −60.432 |
| φ₁ for chain B (°) | −55.668 | −60.480 | −50.045 |
| φ₁ for chain C (°) | −71.797 | −77.250 | −70.284 |
| φ₁ for chain D (°) | −55.818 | −57.605 | −52.207 |
| Δφ₀ for chain B (°) | −76.514 | −74.126 | −76.093 |
| Δφ₀ for chain C (°) | 179.775 | −179.460 | 178.717 |
| Δφ₀ for chain D (°) | 103.584 | 104.022 | 102.380 |
| starting heptad position | b | b | b |
| for chain A | | | |
| starting heptad position | b | b | b |
| for chain B | | | |
| starting heptad position | b | e | b |
| for chain C | | | |
| starting heptad position | b | b | b |
| for chain D | | | |
| pitch (Å) | 215.636 | 179.146 | 166.188 |
| rise per residue (Å) | 1.595 | 1.515 | 1.501 |
| ΔZ_{off} for chain B (Å) | −1.686 | −1.305 | −0.912 |
| ΔZ_{aa′} for chain B (Å) | 3.592 | 3.738 | 4.045 |
| ΔZ_{off} for chain C (Å) | −0.011 | 0.566 | 0.702 |
| ΔZ_{aa′} for chain C (Å) | −0.012 | 0.547 | 0.673 |
| ΔZ_{off} for chain D (Å) | −1.693 | −0.930 | −0.938 |
| ΔZ_{aa′} for chain D (Å) | 3.586 | 4.101 | 4.027 |
| absolute Z_{off} for chain B | 1.175 | 1.701 | 1.922 |
| (Å) | | | |
| absolute Z_{off} for chain C | −0.013 | 0.498 | 0.564 |
| (Å) | | | |
| absolute Z_{off} for chain D | 1.171 | 2.020 | 1.937 |
| (Å) | | | |
| RMSD (Å) | | 0.698 | 0.686 |
| fit CA-CA distance (Å) | 3.78 | 3.78 | 3.75 |

*Parameters were fit using the CCCP server¹⁷: http://arteni.cs.dartmouth.edu/cccp/index.fit.php

TABLE S2
Best-fit Crick parameters* at various stages
of PS1 design (D₂-symmetry constraint)

| Best-fit Crick | Starting | PS1 design | Holo-PS1 centroid |
|---|---|---|---|
| parameters | backbone | backbone | backbone |
| R0 (Å) | 8.020 | 7.748 | 7.903 |
| R1 (Å) | 2.210 | 2.233 | 2.230 |
| ω0 (°/res) | −2.594 | −2.941 | −3.114 |
| ω1 (°/res) | 102.800 | 102.787 | 102.340 |
| α (°) | −13.161 | −15.216 | −16.636 |
| φ1 (°) | −64.125 | −67.241 | −58.392 |
| Δφ0 of Dₙ symmetry (°) | −76.396 | −75.375 | −76.283 |
| starting heptad | B | b | b |
| position | | | |
| pitch (Å) | 215.498 | 178.981 | 166.181 |
| rise per residue (Å) | 1.595 | 1.515 | 1.500 |
| ΔZoff for chain B (Å) | −1.686 | −1.403 | −1.276 |
| ΔZaa′ for chain B (Å) | 3.593 | 3.644 | 3.698 |
| ΔZoff for chain C (Å) | 0.000 | 0.000 | 0.000 |
| ΔZaa′ for chain C (Å) | 0.000 | 0.000 | 0.000 |
| ΔZoff for chain D (Å) | −1.686 | −1.403 | −1.276 |
| ΔZaa′ for chain D (Å) | 3.593 | 3.644 | 3.698 |
| absolute ap zoff (Å) | 1.180 | 1.618 | 1.658 |
| RMSD (Å) | | 0.789 | 0.773 |
| fit CA-CA distance (Å) | 3.75 | 3.75 | 3.73 |

*Parameters were fit using the CCCP server¹⁷: http://arteni.cs.dartmouth.edu/cccp/index.fit.php

TABLE S3
Designed residues of PS1.

| Residue | SCRPZ-2 AA | PS1 AA | Residue | SCRPZ-2 AA | PS1 AA |
|---|---|---|---|---|---|
| number^{a} | identity^{b} | identity | number | identity | identity |
| 3 | L | F | 61 | I | A |
| 6 | L* | L* | 62 | M | A |
| 10 | G* | G* | 65 | G* | G* |
| 13 | I | L | 68 | I | W |
| 14 | L | V | 69 | L | V |
| 16 | I | A | 71 | I | L |
| 17 | A | F | 72 | A | F |
| 20 | V | L | 75 | V | F |
| 23 | I* | I* | 78 | V | A |
| 24 | M | F | 79 | L | I |
| 32 | L* | L* | 87 | L* | L* |
| 35 | L | V | 90 | L* | L* |
| 36 | L* | L* | 91 | I | L |
| 39 | A | I | 94 | A | L |
| 40 | Y | E | 95 | Y | E |
| 42 | L* | L* | 97 | L | A |
| 43 | I* | I* | 98 | I | L |
| 49 | L* | L* | 101 | L | I |
| 50 | A | F | 104 | L* | L* |
| 51 | Y | D | 105 | F | A |

^{a}Residues are numbered according to the expressed 109-residue PS1 protein.
All non ‘*’ residues denotes a mutated residue, and *denotes a retained residue, as shown in FIG. S1.

TABLE S4
Statistics of holo- and apo-PS1 NMR structures

| | Holo-PS1 | Apo-PS1 |
|---|---|---|
| Conformationally-restricting distance constraints | | |
| Intraresidue [i = j] | 451 | 295 |
| Sequential [\|i − j\| = 1] | 499 | 204 |
| Medium Range [1 < \|i − j\| < 5] | 613 | 149 |
| Long Range [\|i − j\| > 5] | 536 | 113 |
| Protein-porphyrin | 26 | — |
| Total | 2125 | 761 |
| Dihedral angle constraints (φ°ψ°) | (96/96) | (96/96) |
| Number of constraints per residue | 19.4 | 7.0 |
| Number of long-range distance constraints per residue | 4.92 | 1.03 |
| CYANA target function [Å²] | 2.67 ± 0.03 | 1.65 ± 0.09 |
| RDC | 89 | — |
| Average number of distance constraints violations per | | |
| CYANA conformer | | |
| 0.2-0.5 Å | 0.0 | 0.0 |
| >0.5 Å | 0.0 | 0.0 |
| Average number of dihedral-angle constraint violations | | |
| per CYANA conformer | | |
| >5° | 0.0 | 0.0 |
| Average RMSD to the mean coordinates [Å] | | |
| Regular secondary structure elements^{a}, backbone heavy | 0.34 ± 0.06 | 1.59 ± 0.43 |
| atoms | | |
| Regular secondary structure elements^{a}, all heavy atoms | 1.16 ± 0.14 | 2.32 ± 0.38 |
| All backbone heavy atoms | 0.78 ± 0.17 | 1.98 ± 0.50 |
| All heavy atoms | 1.62 ± 0.24 | 2.81 ± 0.47 |
| Average RMSD to the model [Å] | 1.05 ± 0.09 | — |
| Ramachandran plot summary [%] | | |
| most favored regions | 99.4 | 98.9 |
| Additionally allowed regions | 0.6 | 1.1 |
| generously allowed regions | 0.0 | 0.0 |
| disallowed regions | 0.0 | 0.0 |
| Overall backbone assignments^{b} | 98.1% | 95.9% |
| Overall side chain chemical shift assignments^{c} | 97.0% | 94.6% |

^{a}Residues 5-26, 29-52, 58-81, 84-106.
^{b}excluding the N-terminal NH₃⁺
^{c}excluding Lys NH₃⁺, Arg NH₂, OH, side chain ¹³CO and aromatic ¹³C^{γ}

TABLE S5
H-D exchange rates and protection factors for
apo and holo-PS1, recorded at pH 6.5 and 298 K.

| | | APO | | | | HOLO | log(PF)ₕₒₗₒ − |
|---|---|---|---|---|---|---|---|
| | | kₑₓ(min⁻¹)* | kᵢₙₜ(min⁻¹)** | log(PF)*** | kₑₓ(min⁻¹) | log(PF) | log(PF)ₐₚₒ |
| S | 1 | N.O. | 2.20E+01 | — | N.O. | — | |
| E | 2 | N.O. | 1.45E+01 | — | N.O. | — | |
| F | 3 | N.O. | 3.91E+00 | — | N.O. | — | |
| E | 4 | >0.3 | 8.35E+00 | — | >0.3 | — | |
| K | 5 | >0.3 | 6.19E+00 | — | N.D. | — | |
| L | 6 | N.D. | 3.25E+00 | — | >0.3 | — | |
| R | 7 | >0.3 | 6.95E+00 | — | >0.3 | — | |
| Q | 8 | >0.3 | 1.79E+01 | — | >0.3 | — | |
| T | 9 | >0.3 | 1.26E+01 | — | >0.3 | — | |
| G | 10 | >0.3 | 2.77E+01 | — | >0.3 | — | |
| D | 11 | >0.3 | 1.75E+01 | — | 4.00E−02 | 6.08 | |
| E | 12 | >0.3 | 4.92E+00 | — | >0.3 | — | |
| L | 13 | 2.25E−03 | 1.79E+00 | 6.68 | 2.07E−02 | 4.46 | −2.22 |
| V | 14 | 1.71E−02 | 1.15E+00 | 4.21 | 3.26E−04 | 8.17 | |
| Q | 15 | N.D. | 7.80E+00 | — | 3.97E−03 | 7.58 | |
| A | 16 | 4.37E−02 | 1.49E+01 | 5.83 | 8.44E−04 | 9.78 | 3.95 |
| F | 17 | 7.98E−03 | 5.39E+00 | 6.52 | >0.3 | — | |
| Q | 18 | 1.85E−02 | 1.24E+01 | 6.51 | 2.78E−03 | 8.40 | 1.89 |
| R | 19 | 2.01E−02 | 1.79E+01 | 6.79 | 9.47E−03 | 7.54 | 0.75 |
| L | 20 | 2.39E−04 | 4.09E+00 | 9.75 | >0.3 | — | |
| R | 21 | 9.90E−03 | 6.95E+00 | 6.55 | 1.34E−02 | 6.25 | −0.30 |
| E | 22 | N.D. | 1.21E+01 | — | 1.63E−02 | 6.61 | |
| I | 23 | 1.43E−02 | 1.26E+00 | 4.48 | 4.49E−04 | 7.94 | 3.46 |
| F | 24 | 1.57E−02 | 3.18E+00 | 5.31 | 5.00E−03 | 6.46 | 1.15 |
| D | 25 | >0.3 | 1.35E+01 | — | >0.3 | — | |
| K | 26 | >0.3 | 5.78E+00 | — | >0.3 | — | |
| G | 27 | >0.3 | 2.30E+01 | — | >0.3 | — | |
| D | 28 | >0.3 | 1.75E+01 | — | >0.3 | — | |
| D | 29 | >0.3 | 7.98E+00 | — | >0.3 | — | |
| D | 30 | >0.3 | 7.98E+00 | — | >0.3 | — | |
| S | 31 | >0.3 | 1.49E+01 | — | >0.3 | — | |
| L | 32 | >0.3 | 4.92E+00 | — | >0.3 | — | |
| E | 33 | >0.3 | 4.49E+00 | — | >0.3 | — | |
| Q | 34 | >0.3 | 7.80E+00 | — | >0.3 | — | |
| V | 35 | >0.3 | 2.96E+00 | — | N.D. | — | |
| L | 36 | >0.3 | 1.79E+00 | — | 2.53E−04 | 8.87 | |
| E | 37 | N.D. | 4.49E+00 | — | 1.91E−03 | 7.76 | |
| E | 38 | 1.89E−03 | 5.27E+00 | 7.93 | 1.03E−03 | 8.55 | 0.62 |
| I | 39 | >0.3 | 1.26E+00 | — | >0.3 | — | |
| E | 40 | >0.3 | 4.28E+00 | — | >0.3 | — | |
| E | 41 | 2.08E−03 | 5.27E+00 | 7.84 | 8.89E−04 | 8.69 | 0.85 |
| L | 42 | >0.3 | 1.79E+00 | — | N.D. | — | |
| I | 43 | >0.3 | 1.08E+00 | — | >0.3 | — | |
| Q | 44 | 2.04E−02 | 6.34E+00 | 5.74 | 1.36E−03 | 8.45 | 2.71 |
| K | 45 | >0.3 | 1.35E+01 | — | 1.45E−02 | 6.84 | |
| H | 46 | >0.3 | 5.91E+01 | — | 1.46E−02 | 8.30 | |
| R | 47 | >0.3 | 5.91E+01 | — | 1.55E−02 | 8.24 | |
| Q | 48 | >0.3 | 1.79E+01 | — | 3.50E−02 | 6.24 | |
| L | 49 | >0.3 | 3.91E+00 | — | >0.3 | — | |
| F | 50 | >0.3 | 3.33E+00 | — | >0.3 | — | |
| D | 51 | >0.3 | 1.35E+01 | — | >0.3 | — | |
| N | 52 | >0.3 | 1.96E+01 | — | >0.3 | — | |
| R | 53 | >0.3 | 2.35E+01 | — | >0.3 | — | |
| Q | 54 | N.D. | 1.79E+01 | — | >0.3 | — | |
| E | 55 | >0.3 | 1.15E+01 | — | >0.3 | — | |
| A | 56 | >0.3 | 6.79E+00 | — | >0.3 | — | |
| A | 57 | >0.3 | 9.37E+00 | — | N.D. | — | |
| D | 58 | >0.3 | 1.18E+01 | — | >0.3 | — | |
| T | 59 | >0.3 | 5.39E+00 | — | N.D. | — | |
| E | 60 | >0.3 | 1.15E+01 | — | >0.3 | — | |
| A | 61 | N.D. | 6.79E+00 | — | N.D. | — | |
| A | 62 | >0.3 | 9.37E+00 | — | >0.3 | — | |
| K | 63 | >0.3 | 8.55E+00 | — | >0.3 | — | |
| Q | 64 | >0.3 | 1.42E+01 | — | >0.3 | — | |
| G | 65 | >0.3 | 2.77E+01 | — | >0.3 | — | |
| D | 66 | N.D. | 1.75E+01 | — | >0.3 | — | |
| Q | 67 | N.O. | 7.28E+00 | — | >0.3 | — | |
| W | 68 | >0.3 | 5.78E+00 | — | 1.94E−02 | 5.70 | |
| V | 69 | N.D. | 1.45E+00 | — | 5.84E−03 | 5.51 | |
| Q | 70 | 3.30E−03 | 7.80E+00 | 7.77 | 1.67E−02 | 6.15 | −1.62 |
| L | 71 | >0.3 | 3.91E+00 | — | 3.00E−03 | 7.17 | |
| F | 72 | 2.67E−02 | 3.33E+00 | 4.83 | 5.01E−04 | 8.80 | 3.97 |
| Q | 73 | >0.3 | 1.24E+01 | — | >0.3 | 8.47 | |
| R | 74 | 1.49E−03 | 1.79E+01 | 9.40 | 1.81E−03 | 9.20 | −0.20 |
| F | 75 | 1.00E−03 | 8.95E+00 | 9.10 | 9.44E−03 | 6.85 | −2.25 |
| R | 76 | 7.88E−03 | 1.29E+01 | 7.40 | 3.42E−03 | 8.24 | 0.84 |
| E | 77 | N.D. | 1.21E+01 | — | 2.96E−03 | 8.32 | |
| A | 78 | 3.80E−03 | 6.79E+00 | 7.49 | 3.23E−03 | 7.65 | 0.16 |
| I | 79 | N.D. | 1.75E+00 | — | 1.64E−02 | 4.67 | |
| D | 80 | N.D. | 6.95E+00 | — | >0.3 | — | |
| K | 81 | >0.3 | 5.78E+00 | — | >0.3 | — | |
| G | 82 | >0.3 | 2.30E+01 | — | >0.3 | — | |
| D | 83 | N.D. | 1.75E+01 | — | >0.3 | — | |
| K | 84 | >0.3 | 5.78E+00 | — | >0.3 | — | |
| D | 85 | N.D. | 1.56E+01 | — | >0.3 | — | |
| S | 86 | >0.3 | 1.49E+01 | — | >0.3 | — | |
| L | 87 | >0.3 | 4.92E+00 | — | >0.3 | — | |
| E | 88 | >0.3 | 4.49E+00 | — | >0.3 | — | |
| Q | 89 | >0.3 | 7.80E+00 | — | >0.3 | — | |
| L | 90 | 8.33E−03 | 3.91E+00 | 6.15 | >0.3 | — | |
| L | 91 | >0.3 | 1.52E+00 | — | 3.22E−04 | 8.46 | |
| E | 92 | 9.96E−03 | 4.49E+00 | 6.11 | >0.3 | — | |
| E | 93 | 5.03E−03 | 5.27E+00 | 6.95 | 7.50E−04 | 8.86 | 1.91 |
| L | 94 | >0.3 | 1.79E+00 | — | 3.28E−03 | 6.30 | |
| E | 95 | >0.3 | 4.49E+00 | — | 1.21E−03 | 8.22 | |
| Q | 96 | 2.67E−02 | 7.80E+00 | 5.68 | 2.00E−03 | 8.27 | 2.59 |
| A | 97 | 3.31E−03 | 1.49E+01 | 8.41 | 5.77E−04 | 10.16 | 1.75 |
| L | 98 | 2.06E−03 | 2.47E+00 | 7.09 | **** | — | |
| Q | 99 | >0.3 | 6.64E+00 | — | 3.23E−02 | 5.33 | |
| K | 100 | >0.3 | 1.35E+01 | — | >0.3 | — | |
| I | 101 | >0.3 | 2.30E+00 | — | 2.48E−03 | 6.83 | |
| R | 102 | >0.3 | 6.64E+00 | — | 1.86E−02 | 5.88 | |
| E | 103 | >0.3 | 1.21E+01 | — | N.D. | — | |
| L | 104 | >0.3 | 1.79E+00 | — | 4.22E−02 | 3.75 | |
| A | 105 | >0.3 | 5.78E+00 | — | >0.3 | — | |
| E | 106 | >0.3 | 7.28E+00 | — | >0.3 | — | |
| K | 107 | >0.3 | 6.19E+00 | — | >0.3 | — | |
| K | 108 | >0.3 | 1.13E+01 | — | >0.3 | — | |
| N | 109 | >0.3 | 3.82E+01 | — | >0.3 | — | |
| W | 68 | >0.3 | 1.40E+01 | — | 1.56E−02 | 2.96 | ***** |

*Observed pseudo-first order rate constant.
**Calculated according to the paper titled Primary structure effects on peptide group hydrogen exchange (Bai et al, proteins, 1993, v17, p75).
***PF = kᵢₙₜ/kₑₓ.
**** The kₑₓ of the L98 can not be obtained from the data recorded in the two-day period, and a comparison to the peak intensity of the same residue in the HSQC spectra recorded in 95% H2O shows that kₑₓ is very slow.
***** W 68 indole HN peak.
N.O.: not observed
N.D.: not determined due to overlap.

TABLE S6
SOCKET knobs into holes packing information: PS1 Design Model

| | knobs in helix 0: | |
|---|---|---|
| 1) | 10 (LEU 12, helix 0) | (hole: ILE 38, LEU 41, ILE 42, HIS 45 helix 1) packing |
| | type 4 | angle 150.536 |
| 2) | 13 (ALA 15, helix 0) | (hole: VAL 34, GLU 37, ILE 38, LEU 41 helix 1) packing |
| | type 4 | angle 69.734 |
| 3) | 17 (LEU 19, helix 0) | (hole: LEU 31, VAL 34, LEU 35, ILE 38 helix 1) packing |
| | type 3 | angle 144.324 |
| 7) | 14 (PHE 16, helix 0) | (hole: LEU 90, LEU 93, GLU 94, LEU 97 helix 3) packing |
| | type 4 | angle 81.812 |
| | knobs in helix 1: | |
| 4) | 30 (VAL 34, helix 1) | (hole: ALA 15, ARG 18, LEU 19, ILE 22 helix 0) packing |
| | type 4 | angle 70.636 |
| 5) | 34 (ILE 38, helix 1) | (hole: LEU 12, ALA 15, PHE 16, LEU 19 helix 0) packing |
| | type 4 | angle 144.625 |
| 6) | 37 (LEU 41, helix 1) | (hole: THR 8, GLU 11, LEU 12, ALA 15 helix 0) packing |
| | type 4 | angle 74.129 |
| 9) | 31 (LEU 35, helix 1) | (hole: PHE 71, PHE 74, ARG 75, ILE 78 helix 2) packing |
| | type 4 | angle 86.327 |
| | knobs in helix 2: | |
| 10) | 61 (PHE 71, helix 2) | (hole: LEU 35, ILE 38, GLU 39, ILE 42 helix 1) packing |
| | type 4 | angle 74.315 |
| 11) | 65 (ARG 75, helix 2) | (hole: GLU 32, LEU 35, GLU 36, GLU 39 helix 1) packing |
| | type 4 | angle 150.436 |
| 15) | 57 (TRP 67, helix 2) | (hole: LEU 93, ALA 96, LEU 97, ILE 100 helix 3) packing |
| | type 3 | angle 139.812 |
| 16) | 60 (LEU 70, helix 2) | (hole: LEU 89, GLU 92, LEU 93, ALA 96 helix 3) packing |
| | type 4 | angle 71.204 |
| 17) | 64 (PHE 74, helix 2) | (hole: LEU 86, LEU 89, LEU 90, LEU 93 helix 3) packing |
| | type 4 | angle 153.293 |
| | knobs in helix 3: | |
| 8) | 74 (LEU 90, helix 3) | (hole: PHE 16, LEU 19, ARG 20, PHE 23 helix 0) packing |
| | type 4 | angle 84.633 |
| 18) | 77 (LEU 93, helix 3) | (hole: TRP 67, LEU 70, PHE 71, PHE 74 helix 2) packing |
| | type 4 | angle 141.983 |
| 19) | 80 (ALA 96, helix 3) | (hole: GLN 63, GLN 66, TRP 67, LEU 70 helix 2) packing |
| | type 4 | angle 70.023 |

holes in helix 0:

ALA 15, ARG 18, LEU 19, ILE 22 (knob: 30 (VAL 34, helix 1))
LEU 12, ALA 15, PHE 16, LEU 19 (knob: 34 (ILE 38, helix 1))
THR 8, GLU 11, LEU 12, ALA 15 (knob: 37 (LEU 41, helix 1))
PHE 16, LEU 19, ARG 20, PHE 23 (knob: 74 (LEU 90, helix 3))

holes in helix 1:

ILE 38, LEU 41, ILE 42, HIS 45 (knob: 10 (LEU 12, helix 0))
VAL 34, GLU 37, ILE 38, LEU 41 (knob: 13 (ALA 15, helix 0))
LEU 31, VAL 34, LEU 35, ILE 38 (knob: 17 (LEU 19, helix 0))
LEU 35, ILE 38, GLU 39, ILE 42 (knob: 61 (PHE 71, helix 2))
GLU 32, LEU 35, GLU 36, GLU 39 (knob: 65 (ARG 75, helix 2))

holes in helix 2:

PHE 71, PHE 74, ARG 75, ILE 78 (knob: 31 (LEU 35, helix 1))
TRP 67, LEU 70, PHE 71, PHE 74 (knob: 77 (LEU 93, helix 3))
GLN 63, GLN 66, TRP 67, LEU 70 (knob: 80 (ALA 96, helix 3))

holes in helix 3:

LEU 90, LEU 93, GLU 94, LEU 97 (knob: 14 (PHE 16, helix 0))
LEU 93, ALA 96, LEU 97, ILE 100 (knob: 57 (TRP 67, helix 2))
LEU 89, GLU 92, LEU 93, ALA 96 (knob: 60 (LEU 70, helix 2))
LEU 86, LEU 89, LEU 90, LEU 93 (knob: 64 (PHE 74, helix 2))

TABLE S7
SOCKET knobs into holes packing information: Apo-PS1 open centroid
knobs in helix 0:

| | |
|---|---|
| 2) 9 (LEU 13, helix 0) | (hole: ILE 39, LEU 42, ILE 43, HIS 46 helix 1) packing |
| type 4 | angle 143.769 |
| 3) 12 (ALA 16, helix 0) | (hole: VAL 35, GLU 38, ILE 39, LEU 42 helix 1) packing |
| type 4 | angle 89.672 |
| 4) 16 (LEU 20, helix 0) | (hole: LEU 32, VAL 35, LEU 36, ILE 39 helix 1) packing |
| type 4 | angle 150.117 |

knobs in helix 1:

| | |
|---|---|
| 5) 29 (VAL 35, helix 1) | (hole: ALA 16, ARG 19, LEU 20, ILE 23 helix 0) packing |
| type 4 | angle 94.618 |
| 6) 33 (ILE 39, helix 1) | (hole: LEU 13, ALA 16, PHE 17, LEU 20 helix 0) packing |
| type 4 | angle 153.438 |
| 7) 36 (LEU 42, helix 1) | (hole: THR 9, GLU 12, LEU 13, ALA 16 helix 0) packing |
| type 4 | angle 78.953 |

knobs in helix 2:

| | |
|---|---|
| 10) 61 (LEU 71, helix 2) | (hole: LEU 90, GLU 93, LEU 94, ALA 97 helix 3) packing |
| type 4 | angle 91.681 |
| 11) 65 (PHE 75, helix 2) | (hole: LEU 87, LEU 90, LEU 91, LEU 94 helix 3) packing |
| type 4 | angle 138.155 |

knobs in helix 3:

| | |
|---|---|
| 12) 78 (LEU 90, helix 3) | (hole: LEU 71, ARG 74, PHE 75, ALA 78 helix 2) packing |
| type 4 | angle 70.633 |
| 13) 82 (LEU 94, helix 3) | (hole: TRP 68, LEU 71, PHE 72, PHE 75 helix 2) packing |
| type 3 | angle 153.471 |

holes in helix 0:

ALA 16, ARG 19, LEU 20, ILE 23 (knob: 29 (VAL 35, helix 1))
LEU 13, ALA 16, PHE 17, LEU 20 (knob: 33 (ILE 39, helix 1))
THR 9, GLU 12, LEU 13, ALA 16 (knob: 36 (LEU 42, helix 1))

holes in helix 1:

ILE 39, LEU 42, ILE 43, HIS 46 (knob: 9 (LEU 13, helix 0))
VAL 35, GLU 38, ILE 39, LEU 42 (knob: 12 (ALA 16, helix 0))
LEU 32, VAL 35, LEU 36, ILE 39 (knob: 16 (LEU 20, helix 0))

holes in helix 2:

LEU 71, ARG 74, PHE 75, ALA 78 (knob: 78 (LEU 90, helix 3))
TRP 68, LEU 71, PHE 72, PHE 75 (knob: 82 (LEU 94, helix 3))

holes in helix 3:

LEU 90, GLU 93, LEU 94, ALA 97 (knob: 61 (LEU 71, helix 2))
LEU 87, LEU 90, LEU 91, LEU 94 (knob: 65 (PHE 75, helix 2))

TABLE S8
SOCKET knobs into holes packing information: Holo-PS1 centroid
knobs in helix 0:

| | |
|---|---|
| 1) 11 (LEU 13, helix 0) | (hole: ILE 39, LEU 42, ILE 43, HIS 46 helix 1) packing |
| type 4 | angle 151.598 |
| 2) 14 (ALA 16, helix 0) | (hole: VAL 35, GLU 38, ILE 39, LEU 42 helix 1) packing |
| type 4 | angle 68.932 |
| 3) 18 (LEU 20, helix 0) | (hole: LEU 32, VAL 35, LEU 36, ILE 39 helix 1) packing |
| type 3 | angle 153.543 |
| 8) 15 (PHE 17, helix 0) | (hole: LEU 91, LEU 94, GLU 95, LEU 98 helix 3) packing |
| type 4 | angle 81.896 |

knobs in helix 1:

| | |
|---|---|
| 4) 30 (VAL 35, helix 1) | (hole: ALA 16, ARG 19, LEU 20, ILE 23 helix 0) packing |
| type 4 | angle 75.308 |
| 5) 34 (ILE 39, helix 1) type | (hole: LEU 13, ALA 16, PHE 17, LEU 20 helix 0) packing |
| 4 | angle 150.056 |
| 6) 37 (LEU 42, helix 1) | (hole: THR 9, GLU 12, LEU 13, ALA 16 helix 0) packing |
| type 4 | angle 75.008 |
| 11) 31 (LEU 36, helix 1) | (hole: PHE 72, PHE 75, ARG 76, ILE 79 helix 2) packing |
| type 3 | angle 92.611 |

knobs in helix 2:

| | |
|---|---|
| 12) 61 (PHE 72, helix 2) | (hole: LEU 36, ILE 39, GLU 40, ILE 43 helix 1) packing |
| type 4 | angle 74.018 |
| 13)65 (ARG 76, helix 2) | (hole: GLU 33, LEU 36, GLU 37, GLU 40 helix 1) packing |
| type 3 | angle 160.877 |
| 15) 57 (TRP 68, helix 2) | (hole: LEU 94, ALA 97, LEU 98, ILE 101 helix 3) packing |
| type 3 | angle 139.833 |
| 16) 60 (LEU 71, helix 2) | (hole: LEU 90, GLU 93, LEU 94, ALA 97 helix 3) packing |
| type 4 | angle 62.249 |
| 17) 64 (PHE 75, helix 2) | (hole: LEU 87, LEU 90, LEU 91, LEU 94 helix 3) packing |
| type 4 | angle 146.054 |

knobs in helix 3:

| | |
|---|---|
| 10) 77 (LEU 91, helix 3) | (hole: PHE 17, LEU 20, ARG 21, PHE 24 helix 0) packing |
| type 4 | angle 104.380 |
| 18) 76 (LEU 90, helix 3) | (hole: LEU 71, ARG 74, PHE 75, ALA 78 helix 2) packing |
| type 4 | angle 60.542 |
| 19) 80 (LEU 94, helix 3) | (hole: TRP 68, LEU 71, PHE 72, PHE 75 helix 2) packing |
| type 4 | angle 142.763 |
| 20) 83 (ALA 97, helix 3) | (hole: GLN 64, GLN 67, TRP 68, LEU 71 helix 2) packing |
| type 4 | angle 63.787 |

holes in helix 0:

ALA 16, ARG 19, LEU 20, ILE 23 (knob: 30 (VAL 35, helix 1))
LEU 13, ALA 16, PHE 17, LEU 20 (knob: 34 (ILE 39, helix 1))
THR 9, GLU 12, LEU 13, ALA 16 (knob: 37 (LEU 42, helix 1))
PHE 17, LEU 20, ARG 21, PHE 24 (knob: 77 (LEU 91, helix 3))

holes in helix 1:

ILE 39, LEU 42, ILE 43, HIS 46 (knob: 11 (LEU 13, helix 0))
VAL 35, GLU 38, ILE 39, LEU 42 (knob: 14 (ALA 16, helix 0))
LEU 32, VAL 35, LEU 36, ILE 39 (knob: 18 (LEU 20, helix 0))
LEU 36, ILE 39, GLU 40, ILE 43 (knob: 61 (PHE 72, helix 2))
GLU 33, LEU 36, GLU 37, GLU 40 (knob: 65 (ARG 76, helix 2))

holes in helix 2:

PHE 72, PHE 75, ARG 76, ILE 79 (knob: 31 (LEU 36, helix 1))
LEU 71, ARG 74, PHE 75, ALA 78 (knob: 76 (LEU 90, helix 3))
TRP 68, LEU 71, PHE 72, PHE 75 (knob: 80 (LEU 94, helix 3))
GLN 64, GLN 67, TRP 68, LEU 71 (knob: 83 (ALA 97, helix 3))

holes in helix 3:

LEU 91, LEU 94, GLU 95, LEU 98 (knob: 15 (PHE 17, helix 0))
LEU 94, ALA 97, LEU 98, ILE 101 (knob: 57 (TRP 68, helix 2))
LEU 90, GLU 93, LEU 94, ALA 97 (knob: 60 (LEU 71, helix 2))
LEU 87, LEU 90, LEU 91, LEU 94 (knob: 64 (PHE 75, helix 2))

### Command Line and Input Files

Input files and command lines for design calculations.

Command lines and flags for generating the backbone ensemble via Rosetta backrub

Flags
-nstruct 200
-constraints:cst_fa_file my_atomic.cst
-constraints:cst_fa_weight 1
-extrachi_cutoff 0
-ex 1
-ex2
-backrub:mc_kt 0.8
-backrub:ntrials 10000
-backrub:sc_prob_withinrot 0.1
-backrub:initial_pack
-backrub:mm_bend_weight 2
-backrub:pivot_residues 1-108

Command Line
˜/rosetta/rosetta-3.5/rosetta_source/bin/backrub.default.linuxgccrelease-database
˜/rosetta/rosetta-3.5/rosetta_database/-s holo_input_model.pdb@flags.txt-extra_res_fa PZNF.params

Command lines, RosettaScript, and flags for the flexible backbone sequence design protocol.

RosettaScript

<dock_design>

<SCOREFXNS>

<scorewts weights=score13>

<Reweight scoretype = atom_pair_constraint weight = 1/>
<Reweight scoretype = angle_constraint weight = 1/>
<Reweight scoretype = hack_aro weight = 1/>
<Reweight scoretype = fa_pair weight = 0/>
<Reweight scoretype = hack_elec weight = 0.55/>
<Reweight scoretype = rg weight = 2/>

</scorewts>
<scorewts_backrub weights=score13>

<Reweight scoretype = atom_pair_constraint weight = 1/>
<Reweight scoretype = angle_constraint weight = 1/>
<Reweight scoretype = rg weight = 2/>
<Reweight scoretype = hack_aro weight = 1/>

</scorewts_backrub>
<softwts weights =soft_rep_design>

<Reweight scoretype = atom_pair_constraint weight = 1/>
<Reweight scoretype = angle_constraint weight = 1/>
<Reweight scoretype = hack_aro weight = 1/>
<Reweight scoretype = rg weight = 2/>

</softwts>

</SCOREFXNS>
<FILTERS>

<PackStat name = pstat threshold = 0.58 repeats = 3/>

</FILTERS>
<TASKOPERATIONS>

<ReadResfile name = rr filename = resfile.txt/>
<InitializeFromCommandline name = ifcl/>
<IncludeCurrent name = input_sc/>
<RestrictToRepacking name = no_mutations/>
<ExtraRotamersGeneric name = extra_rot1 ex1 = 1 ex2 = 1
ex1_sample_level = 3
ex2_sample_level = 3 extrachi_cutoff = 0/>
<OperateOnCertainResidues name = fixpolars>

<PreventRepackingRLT/>
<ResidueHasProperty property = POLAR/>

</OperateOnCertainResidues>
<OperateOnCertainResidues name = fixcharged>

<PreventRepackingRLT/>
<ResidueHasProperty property = CHARGED/>

</OperateOnCertainResidues>

</TASKOPERATIONS>
<MOVERS>

<ConstraintSetMover name = atomic cst_file =
my_atomic.cst/>
<PackRotamersMover name = repack scorefxn = scorewts
task_operations =

ifcl,no_mutations/>

<PackRotamersMover name = pr1 scorefxn = softwts
task_operations = rr,ifcl/>
<PackRotamersMover name = pr2 scorefxn = scorewts
task_operations =

rr,ifcl,extra_rot1/>

<MinMover name=minmovsc scorefxn = softwts tolerance =
0.005 chi=1 bb=0/>
<MinMover name=minmovbb scorefxn = scorewts tolerance =
0.005 chi=0

bb=1/>

<Backrub name = backrub pivot_residues=1-108
require_mm_bend =1/>
<Sidechain name = sidechain task_operations =

ifcl,no_mutations,fixpolars,fixcharged/>

<ParsedProtocol name = backrub_protocol mode =
single_random>

<Add mover_name = backrub apply_probability = 0.75/>
<Add mover_name = sidechain apply_probability =
0.25/>

</ParsedProtocol>
<GenericMonteCarlo name = backrub_mc mover_name =
backrub_protocol
scorefxn_name = scorewts_backrub trials = 200 temperature =
1.2 preapply = 0/>
<ParsedProtocol name=flexdes>

<Add mover_name=pr1/>
<Add mover_name=minmovsc/>
<Add mover_name=pr2/>
<Add mover_name=minmovbb/>
<Add mover_name=pr2 filter_name=pstat/>

</ParsedProtocol>
<GenericMonteCarlo name=iterate mover_name=flexdes
scorefxn_name=scorewts trials=3 preapply=0
temperature =0.4/>

</MOVERS>
<OUTPUT scorefxn=scorewts/>
<APPLY_TO_POSE>
</APPLY_TO_POSE>
<PROTOCOLS>

<Add mover_name=atomic/>
<Add mover_name=repack/>
<Add mover_name=backrub_mc/>
<Add mover_name = iterate/>
<Add filter name = pstat/>

</PROTOCOLS>

</dock_design>

Contents of Constraint File (my_atomic.cst):
AtomPair NE2 45 Å ZN1 1X HARMONIC 2.0 0.1
Angle ZN1 1X NE2 45 Å ND1 45 Å CIRCULARHARMONIC 2.806 0.2
Angle ZN1 1X NE2 45 Å CG 45 Å CIRCULARHARMONIC 2.845 0.2

Flags
-parser:protocol RosettaScript.xml
-nstruct 500
-out:file:fullatom
-out:pdb
-packing:multi_cool_annealer 10
-packing:linmem_ig 20

Command Line Input
/rosetta/rosetta-3.5/rosetta_source/bin/rosetta scripts. default.linuxgccrelease-database
/rosetta/rosetta-3.5/rosetta_database/-s . . . /holo_input_model.pdb-extra_res_fa PZNF.params@flags.txt

Contents of the Residue File (resfile.txt):

| NATRO | | | | |
|---|---|---|---|---|
| USE_INPUT_SC | | | | |
| start | | | | |
| 2 | A | APOLAR | NOTAA | WYCMH |
| 5 | A | APOLAR | NOTAA | WYCMH |
| 6 | A | NATAA | | |
| 8 | A | NATAA | | |
| 9 | A | APOLAR | NOTAA | WYCMH |
| 10 | A | NATAA | | |
| 12 | A | APOLAR | NOTAA | WYCMH |
| 13 | A | APOLAR | NOTAA | WYCMH |
| 15 | A | APOLAR | NOTAA | WYCMH |
| 16 | A | APOLAR | NOTAA | WYCMH |
| 19 | A | APOLAR | NOTAA | WYCMH |
| 22 | A | APOLAR | NOTAA | WYCMH |
| 23 | A | APOLAR | NOTAA | WYCMH |
| 31 | A | APOLAR | NOTAA | WYCMH |
| 34 | A | APOLAR | NOTAA | WYCMH |
| 35 | A | APOLAR | NOTAA | WYCMH |
| 38 | A | APOLAR | NOTAA | WYCMH |
| 39 | A | ALLAAxc | NOTAA | WYCMH |
| 41 | A | APOLAR | NOTAA | WYCMH |
| 42 | A | APOLAR | NOTAA | WYCMH |
| 45 | A | NATRO | | |
| 46 | A | NATAA | | |
| 48 | A | APOLAR | NOTAA | WYCMH |
| 49 | A | APOLAR | NOTAA | WYCMH |
| 50 | A | ALLAAxc | NOTAA | WYCMH |
| 60 | A | APOLAR | NOTAA | WYCMH |
| 61 | A | APOLAR | NOTAA | WYCMH |
| 64 | A | APOLAR | NOTAA | WYCMH |
| 65 | A | NATAA | | |
| 67 | A | PIKAA | W | |
| 68 | A | APOLAR | NOTAA | WYCMH |
| 70 | A | APOLAR | NOTAA | WYCMH |
| 71 | A | APOLAR | NOTAA | WYCMH |
| 74 | A | APOLAR | NOTAA | WYCMH |
| 77 | A | APOLAR | NOTAA | WYCMH |
| 78 | A | APOLAR | NOTAA | WYCMH |
| 86 | A | APOLAR | NOTAA | WYCMH |
| 89 | A | APOLAR | NOTAA | WYCMH |
| 90 | A | APOLAR | NOTAA | WYCMH |
| 93 | A | APOLAR | NOTAA | WYCMH |
| 94 | A | ALLAAxc | NOTAA | WYCMH |
| 96 | A | APOLAR | NOTAA | WYCMH |
| 97 | A | APOLAR | NOTAA | WYCMH |
| 100 | A | APOLAR | NOTAA | WYCMH |
| 101 | A | NATAA | | |
| 103 | A | APOLAR | NOTAA | WYCMH |
| 104 | A | APOLAR | NOTAA | WYCMH |
| 105 | A | NATAA | | |

Contents of (CF₃)₄PZn parameters file (PZNF.params):

| NAMEPZF | | | | |
|---|---|---|---|---|
| IO_STRING PZF Z | | | | |
| TYPE LIGAND | | | | |
| AA UNK | | | | |
| ATOM | ZN1 | Zn2p | X | 1.01 |
| ATOM | N1 | Npro | X | −0.65 |
| ATOM | C18 | aroC | X | 0.42 |
| ATOM | C17 | aroC | X | −0.37 |
| ATOM | C16 | aroC | X | 0.46 |
| ATOM | N4 | Npro | X | −0.66 |
| ATOM | C13 | aroC | X | 0.51 |
| ATOM | C12 | aroC | X | −0.44 |
| ATOM | C11 | aroC | X | 0.46 |
| ATOM | N3 | Npro | X | −0.65 |
| ATOM | C8 | aroC | X | 0.43 |
| ATOM | C7 | aroC | X | −0.39 |
| ATOM | C6 | aroC | X | 0.47 |
| ATOM | N2 | Npro | X | −0.67 |
| ATOM | C3 | aroC | X | 0.53 |
| ATOM | C2 | aroC | X | −0.44 |
| ATOM | C1 | aroC | X | 0.47 |
| ATOM | C20 | aroC | X | −0.27 |
| ATOM | C19 | aroC | X | −0.27 |
| ATOM | H6 | Haro | X | 0.19 |
| ATOM | H7 | Haro | X | 0.19 |
| ATOM | C21 | CH1 | X | 0.50 |
| ATOM | F1 | F | X | −0.16 |
| ATOM | F2 | F | X | −0.17 |
| ATOM | F10 | F | X | −0.17 |
| ATOM | C4 | aroC | X | −0.30 |
| ATOM | C5 | aroC | X | −0.29 |
| ATOM | H2 | Haro | X | 0.20 |
| ATOM | H1 | Haro | X | 0.20 |
| ATOM | C22 | CH1 | X | 0.52 |
| ATOM | F7 | F | X | −0.17 |
| ATOM | F8 | F | X | −0.17 |
| ATOM | F9 | F | X | −0.18 |
| ATOM | C9 | aroC | X | −0.28 |
| ATOM | C10 | aroC | X | −0.26 |
| ATOM | H8 | Haro | X | 0.19 |
| ATOM | H3 | Haro | X | 0.19 |
| ATOM | C23 | CH1 | X | 0.50 |
| ATOM | F5 | F | X | −0.16 |
| ATOM | F6 | F | X | −0.17 |
| ATOM | F12 | F | X | −0.17 |
| ATOM | C14 | aroC | X | −0.29 |
| ATOM | C15 | aroC | X | −0.29 |
| ATOM | H5 | Haro | X | 0.20 |
| ATOM | H4 | Haro | X | 0.19 |
| ATOM | C24 | CH1 | X | 0.53 |
| ATOM | F11 | F | X | −0.19 |
| ATOM | F3 | F | X | −0.17 |
| ATOM | F4 | F | X | −0.17 |
| BOND | ZN1 | N1 | | |
| BOND | ZN1 | N3 | | |
| BOND | ZN1 | N4 | | |
| BOND | ZN1 | N2 | | |
| BOND | C24 | F11 | | |
| BOND | F1 | C21 | | |
| BOND | F2 | C21 | | |
| BOND | F3 | C24 | | |
| BOND | F4 | C24 | | |
| BOND | F5 | C23 | | |
| BOND | F6 | C23 | | |
| BOND | F7 | C22 | | |
| BOND | F8 | C22 | | |
| BOND | N1 | C18 | | |
| BOND | N1 | C1 | | |
| BOND | N2 | C6 | | |
| BOND | N2 | C3 | | |
| BOND | N3 | C8 | | |
| BOND | N3 | C11 | | |
| BOND | N4 | C16 | | |
| BOND | N4 | C13 | | |
| BOND | C1 | C20 | | |
| BOND | C1 | C2 | | |
| BOND | C2 | C3 | | |
| BOND | C2 | C21 | | |
| BOND | C3 | C4 | | |
| BOND | C4 | C5 | | |
| BOND | C4 | H1 | | |
| BOND | C5 | H2 | | |
| BOND | C5 | C6 | | |
| BOND | C6 | C7 | | |
| BOND | C7 | C22 | | |
| BOND | C7 | C8 | | |
| BOND | C8 | C9 | | |
| BOND | C9 | H3 | | |
| BOND | C9 | C10 | | |
| BOND | C10 | H8 | | |
| BOND | C10 | C11 | | |
| BOND | C11 | C12 | | |
| BOND | C12 | C13 | | |
| BOND | C12 | C23 | | |
| BOND | C13 | C14 | | |
| BOND | C14 | C15 | | |
| BOND | C14 | H4 | | |
| BOND | C15 | H5 | | |
| BOND | C15 | C16 | | |
| BOND | C16 | C17 | | |
| BOND | C17 | C24 | | |
| BOND | C17 | C18 | | |
| BOND | C18 | C19 | | |
| BOND | C19 | H6 | | |
| BOND | C19 | C20 | | |
| BOND | C20 | H7 | | |
| BOND | C21 | F10 | | |
| BOND | C22 | F9 | | |
| BOND | C23 | F12 | | |
| CHI 1 | C3 | C2 | C21 | F1 |
| CHI 2 | C8 | C7 | C22 | F7 |
| CHI 3 | C13 | C12 | C23 | F5 |
| CHI 4 | C18 | C17 | C24 | F11 |

NBR_ATOM ZN1
NBR_RADIUS 6.387332

| | | | | | | | |
|---|---|---|---|---|---|---|---|
| ICOOR_INTERNAL | ZN1 | 0.000000 | 0.000000 | 0.000000 | ZN1 | N1 | C18 |
| ICOOR_INTERNAL | N1 | 0.000000 | 180.000000 | 2.064355 | ZN1 | N1 | C18 |
| ICOOR_INTERNAL | C18 | 0.000001 | 51.447825 | 1.369076 | N1 | ZN1 | C18 |
| ICOOR_INTERNAL | C17 | 6.625837 | 54.661320 | 1.412896 | C18 | N1 | ZN1 |
| ICOOR_INTERNAL | C16 | −8.838188 | 54.513694 | 1.411530 | C17 | C18 | N1 |
| ICOOR_INTERNAL | N4 | 8.385056 | 56.245312 | 1.374326 | C16 | C17 | C18 |
| ICOOR_INTERNAL | C13 | −177.426793 | 72.629665 | 1.370374 | N4 | C16 | C17 |
| ICOOR_INTERNAL | C12 | −172.597100 | 55.764058 | 1.412488 | C13 | N4 | C16 |
| ICOOR_INTERNAL | C11 | 15.118869 | 55.057460 | 1.410234 | C12 | C13 | N4 |
| ICOOR_INTERNAL | N3 | −15.071141 | 55.251686 | 1.368644 | C11 | C12 | C13 |
| ICOOR_INTERNAL | C8 | 174.454044 | 72.748278 | 1.368355 | N3 | C11 | C12 |
| ICOOR_INTERNAL | C7 | 175.549092 | 54.651816 | 1.412704 | C8 | N3 | C11 |
| ICOOR_INTERNAL | C6 | −8.949997 | 54.548075 | 1.411877 | C7 | C8 | N3 |
| ICOOR_INTERNAL | N2 | 8.726143 | 56.164530 | 1.373619 | C6 | C7 | C8 |
| ICOOR_INTERNAL | C3 | −177.455606 | 72.629643 | 1.370275 | N2 | C6 | C7 |
| ICOOR_INTERNAL | C2 | −172.440358 | 55.835300 | 1.411183 | C3 | N2 | C6 |
| ICOOR_INTERNAL | C1 | 14.746094 | 55.058826 | 1.411413 | C2 | C3 | N2 |
| ICOOR_INTERNAL | C20 | 159.351269 | 54.572579 | 1.451463 | C1 | C2 | C3 |
| ICOOR_INTERNAL | C19 | −173.134442 | 73.124901 | 1.356577 | C20 | C1 | C2 |
| ICOOR_INTERNAL | H6 | 178.675786 | 53.211637 | 1.077487 | C19 | C20 | C1 |
| ICOOR_INTERNAL | H7 | 177.242610 | 53.864408 | 1.078340 | C20 | C1 | C19 |
| ICOOR_INTERNAL | C21 | 172.161545 | 61.209306 | 1.518010 | C2 | C3 | C1 |
| ICOOR_INTERNAL | F1 | 26.588574 | 66.722458 | 1.351871 | C21 | C2 | C3 |
| ICOOR_INTERNAL | F2 | 118.742085 | 68.391753 | 1.355075 | C21 | C2 | F1 |
| ICOOR_INTERNAL | F10 | 119.896471 | 67.475489 | 1.355382 | C21 | C2 | F2 |
| ICOOR_INTERNAL | C4 | 174.106305 | 70.610058 | 1.451470 | C3 | N2 | C2 |
| ICOOR_INTERNAL | C5 | −2.613059 | 73.025475 | 1.356111 | C4 | C3 | N2 |
| ICOOR_INTERNAL | H2 | −178.723869 | 53.755333 | 1.075472 | C5 | C4 | C3 |
| ICOOR_INTERNAL | H1 | −177.048251 | 53.662204 | 1.077885 | C4 | C3 | C5 |
| ICOOR_INTERNAL | C22 | −176.097888 | 65.736086 | 1.522118 | C7 | C8 | C6 |
| ICOOR_INTERNAL | F7 | −48.708509 | 68.833518 | 1.355195 | C22 | C7 | C8 |
| ICOOR_INTERNAL | F8 | −119.427444 | 65.177131 | 1.348034 | C22 | C7 | F7 |
| ICOOR_INTERNAL | F9 | −121.529899 | 68.269409 | 1.357705 | C22 | C7 | F8 |
| ICOOR_INTERNAL | C9 | −176.602426 | 70.718433 | 1.455487 | C8 | N3 | C7 |
| ICOOR_INTERNAL | C10 | 2.316401 | 72.970357 | 1.356477 | C9 | C8 | N3 |
| ICOOR_INTERNAL | H8 | −179.770544 | 53.066131 | 1.077420 | C10 | C9 | C8 |
| ICOOR_INTERNAL | H3 | 178.815847 | 53.777613 | 1.077761 | C9 | C8 | C10 |
| ICOOR_INTERNAL | C23 | 171.964149 | 61.628846 | 1.518169 | C12 | C13 | C11 |
| ICOOR_INTERNAL | F5 | 28.592915 | 66.966988 | 1.353389 | C23 | C12 | C13 |
| ICOOR_INTERNAL | F6 | 118.547014 | 68.227339 | 1.355152 | C23 | C12 | F5 |
| ICOOR_INTERNAL | F12 | 120.209500 | 67.387458 | 1.354153 | C23 | C12 | F6 |
| ICOOR_INTERNAL | C14 | 174.179256 | 70.623080 | 1.451196 | C13 | N4 | C12 |
| ICOOR_INTERNAL | C15 | −2.538511 | 73.008118 | 1.356972 | C14 | C13 | N4 |
| ICOOR_INTERNAL | H5 | −178.625691 | 53.835483 | 1.074947 | C15 | C14 | C13 |
| ICOOR_INTERNAL | H4 | −176.963403 | 53.769411 | 1.077427 | C14 | C13 | C15 |
| ICOOR_INTERNAL | C24 | −176.025368 | 65.801932 | 1.521861 | C17 | C18 | C16 |
| ICOOR_INTERNAL | F11 | 70.140257 | 68.312105 | 1.358482 | C24 | C17 | C18 |
| ICOOR_INTERNAL | F3 | −119.046543 | 68.720707 | 1.354553 | C24 | C17 | F11 |
| ICOOR_INTERNAL | F4 | −119.508856 | 65.139031 | 1.348886 | C24 | C17 | F3 |

### REFERENCES FOR EXAMPLE 2

1. North, B., Summa, C. M., Ghirlanda, G. & DeGrado, W. F. Dₙ-symmetrical tertiary templates for the design of tubular proteins. J. Mol. Biol. 311, 1081-1090 (2001). 2. Ghirlanda, G. et al. De novo design of a D₂-symmetrical protein that reproduces the diheme four-helix bundle in cytochrome bc₁. J. Am. Chem. Soc. 126, 8141-8147 (2004). 3. Lahr, S. J. et al. Analysis and design of turns in α-helical hairpins. J. Mol. Biol. 346, 1441-1454 (2005). 4. Bender, G. M. et al. De novo design of a single-chain diphenylporphyrin metalloprotein. J. Am. Chem. Soc. 129, 10732-10740 (2007). 5. Fry, H. C. et al. Computational de novo design and characterization of a protein that selectively binds a highly hyperpolarizable abiological chromophore. J. Am. Chem. Soc. 135, 13914-13926 (2013). 6. Davis, I. W., Arendall Iii, W. B., Richardson, D. C. & Richardson, J. S. The backrub motion: How protein backbone shrugs when a sidechain dances. Structure 14, 265-274 (2006). 7. Friedland, G. D., Lakomek, N.-A., Griesinger, C., Meiler, J. & Kortemme, T. A correspondence between solution-state dynamics of an individual protein and the sequence and conformational diversity of its family. PLoS Comput Biol 5, e1000393 (2009). 8. Bradley, P., Misura, K. M. S. & Baker, D. Toward high-resolution de novo structure prediction for small proteins. Science 309, 1868 (2005). 9. Polizzi, N. F. et al. Photoinduced Electron Transfer Elicits a Change in the Static Dielectric Constant of a de Novo Designed Protein. J. Am. Chem. Soc. 138, 2130-2133 (2016). 10. Goll, J. G., Moore, K. T., Ghosh, A. & Therien, M. J. Synthesis, structure, electronic spectroscopy, photophysics, electrochemistry, and x-ray photoelectron spectroscopy of highly-electron-deficient [5,10,15,20-tetrakis(perfluoroalkyl)porphinato]zinc(II) complexes and their free base derivatives. J. Am. Chem. Soc. 118, 8344-8354 (1996). 11. Schrodinger, LLC. The PyMOL Molecular Graphics System, Version 1.8. (2015). 12. Jorgensen, W. L., Chandrasekhar, J., Madura, J. D., Impey, R. W. & Klein, M. L. Comparison of simple potential functions for simulating liquid water. J. Chem. Phys. 79, 926-935 (1983). 13. Harvey, M. J., Giupponi, G. & Fabritiis, G. D. ACEMD: Accelerating Biomolecular Dynamics in the Microsecond Time Scale. J. Chem. Theory Comput. 5, 1632-1639 (2009). 14. Walshaw, J. & Woolfson, D. N. SOCKET: a program for identifying and analysing coiled-coil motifs within protein structures. J. Mol. Biol. 307, 1427-1450 (2001). 15. Hayes, D., Laue, T. & Philo, J. Program Sednterp: sedimentation interpretation program. Durham, N.H.: University of New Hampshire (1995). 16. Moore, K. T., Fletcher, J. T. & Therien, M. J. Syntheses, NMR and EPR Spectroscopy, Electrochemical Properties, and Structural Studies of [5,10,15,20-Tetrakis(perfluoroalkyl)porphinato]iron(II) and -iron(III) Complexes. J. Am. Chem. Soc. 121, 5196-5209 (1999). 17. Grigoryan, G. & DeGrado, W. F. Probing designability via a generalized model of helical bundle geometry. J. Mol. Biol. 405, 1079-1100 (2011).

1. A computer-implemented method, comprising:
(a) identifying a set of ligand binding amino acid residues within a protein for binding to a ligand, wherein each ligand binding amino acid residue within said protein is associated with a set of ligand binding amino acid residue atomic coordinates and each atom of said ligand is associated with a set of ligand atomic coordinates;
(b) identifying a set of core amino acid residues within said protein that do not bind to said ligand, each core amino acid residue within said protein is associated with a set of core amino acid residue atomic coordinates; and
(c) optimizing
said set of ligand binding amino acid residues;
said set of ligand binding amino acid residue atomic coordinates;
said set of core amino acid residues; and
said set of core amino acid residue atomic coordinates;
wherein the optimization is performed using at least an energy minimization calculation, and wherein the optimization is performed to energetically stabilize said protein.

2. The method of claim 1, wherein step c) comprises simultaneously optimizing
said set of ligand binding amino acid residues;
said set of ligand binding amino acid residue atomic coordinates;
said set of core amino acid residues; and
said set of core amino acid residue atomic coordinates.

3. The method of claim 1, wherein the energy minimization calculation comprises a molecular mechanics function, a structural bioinformatics function, an amino acid sidechain packing function, a protein radius of gyration function, or a combination thereof.

4. The method of claim 1, wherein the core amino acids are at least 75% inaccessible to a 1.8 Å spherical probe.

5. The method of claim 1, wherein said set of core amino acids comprises at least six amino acid residues.

6. The method of claim 1, wherein the optimizing comprises fixing an atomic coordinate of at least one ligand binding amino acid residue atomic coordinate; fixing an atomic coordinate of at least one ligand atomic coordinate; prohibiting introduction of an additional amino acid residue into the set of ligand binding amino acid residues; or prohibiting the deletion of an amino acid residue from the set of ligand binding amino acid residues.

7. The method of claim 1, wherein the optimizing comprises fixing at least one atomic coordinate of the ligand atomic coordinates.

8. The method of claim 1, wherein the energy minimization calculation comprises a penalty function.

9. The method of claim 1, wherein the optimizing does not comprise fixing at least one atomic coordinate of at least one core amino acid residue atomic coordinates.

10. The method of claim 1, wherein the optimizing comprises introducing an additional ligand binding amino acid residue into the set of ligand binding amino acid residues, deleting a ligand binding amino acid residue from the set of ligand binding amino acid residues, a geometric transformation of at least one atomic coordinate of the ligand binding amino acid residue atomic coordinates.

11. The method of claim 10, wherein the geometric transformation comprises a translation or a rotation of at least one atomic coordinate of the ligand binding amino acid residue atomic coordinates.

12. The method of claim 1, wherein the optimizing comprises a geometric transformation of at least one atomic coordinate of the core amino acid residue atomic coordinates.

13. The method of claim 12, wherein the geometric transformation of at least one atomic coordinate comprises no greater than a 6 Å displacement of any atomic coordinate.

14. (canceled)

15. The method of claim 1, wherein the optimizing comprises:
(i) an iterative or heuristic algorithm;
(ii) a simplex algorithm, memetic algorithm, differential evolution algorithm, evolutionary algorithm, genetic algorithm, tabu algorithm, particle swarm algorithm, or stimulated annealing algorithm; or
(iii) a Monte Carlo sampling algorithm, dead-end elimination algorithm, branch and bound algorithm, or a pruning algorithm.

16-17. (canceled)

18. The method of claim 1, wherein the ligand is a porphyrin, porphycene, rubyrin, rosarin, hexaphyrin, sapphyrin, chlorophyll, chlorin, phthalocyanine, porphyrazine, corrole, N-confused porphyrin, bacteriochlorophyll, pheophytin, texaphyrin, a detectable agent, a catalyst, a therapeutic agent, biological agent, cytotoxic agent, magnetic resonance imaging agent, positron emission tomography agent, radiological imaging agent, diagnostic agent, theranostic, or a photodynamic therapy agent.

19-23. (canceled)

24. A system, comprising:
at least one data processor; and
at least one memory storing instructions which, when executed by the at least one data processor, result in operations comprising:
(a) identifying a set of ligand binding amino acid residues within a protein for binding to a ligand, wherein each ligand binding amino acid residue within said protein is associated with a set of ligand binding amino acid residue atomic coordinates and each atom of said ligand is associated with a set of ligand atomic coordinates;
(b) identifying a set of core amino acid residues within said protein that do not bind to said ligand, each core amino acid residue within said protein is associated with a set of core amino acid residue atomic coordinates; and
(c) optimizing
said set of ligand binding amino acid residues;
said set of ligand binding amino acid residue atomic coordinates;
said set of core amino acid residues; and
said set of core amino acid residue atomic coordinates;
wherein the optimization is performed using at least an energy minimization calculation, and wherein the optimization is performed to energetically stabilize said protein.

25-39. (canceled)

40. A non-transitory computer-readable storage medium including program code, which when executed by at least one data processor, causes operations comprising:
(a) identifying a set of ligand binding amino acid residues within a protein for binding to a ligand, wherein each ligand binding amino acid residue within said protein is associated with a set of ligand binding amino acid residue atomic coordinates and each atom of said ligand is associated with a set of ligand atomic coordinates;
(b) identifying a set of core amino acid residues within said protein that do not bind to said ligand, each core amino acid residue within said protein is associated with a set of core amino acid residue atomic coordinates; and
(c) optimizing
said set of ligand binding amino acid residues;
said set of ligand binding amino acid residue atomic coordinates;
said set of core amino acid residues; and
said set of core amino acid residue atomic coordinates;
wherein the optimization is performed using at least an energy minimization calculation, and wherein the optimization is performed to energetically stabilize said protein.

41. A protein sequence obtainable based on the energy minimization calculation using the method of claim 1.

42. A protein having an amino acid sequence that is at least 90% identical to SEQ ID NO:1.

43-45. (canceled)

46. The protein of claim 42, wherein the protein is bound to a ligand selected from the group consisting of a porphyrin, porphycene, rubyrin, rosarin, hexaphyrin, sapphyrin, chlorophyll, chlorin, phthalocyanine, porphyrazine, corrole, N-confused porphyrin, bacteriochlorophyll, pheophytin, texaphyrin, a detectable agent, a catalyst, a therapeutic agent, biological agent, cytotoxic agent, magnetic resonance imaging agent, positron emission tomography agent, radiological imaging agent, diagnostic agent, theranostic, and a photodynamic therapy agent.

47-51. (canceled)


SEQUENCE LISTING (8 sequences)

SEQ ID NO: 1 - protein, 108 aa, Artificial sequence
  Synthetic polypeptide
EFEKLRQTGDELVQAFQRLREIFDKGDDDSLEQVLEEIEELIQKHRQLFDNRQEAADTEAAKQGDQWVQLFQRFREAIDKGDKDSLEQLLEELEQALQKIRELAEKKN

SEQ ID NO: 2 - protein, 109 aa, Artificial sequence
  Synthetic polypeptide
SEFEKLRQTGDELVQAFQRLREIFDKGDDDSLEQVLEEIEELIQKHRQLFDNRQEAADTEAAKQGDQWVQLFQRFREAIDKGDKDSLEQLLEELEQALQKIRELAEKKN

SEQ ID NO: 3 - DNA, 378 bp, Artificial sequence
  Synthetic polynucleotide
catatgcatcaccatcaccatcacgaaaacctgtattttcagagcgaattcgaaaaactgcgtcaaaccggcgacgaactggtgcaggcatttcaacgtctgcgcgaaattttcgataaaggtgatgacgatagtctggaacaggttctggaagaaattgaagaactgatccagaaacatcgtcaactgtttgacaatcgccaggaagcggccgatacggaagcagctaaacagggcgaccaatgggtccagctgtttcaacgtttccgcgaagccattgataaaggtgacaaagatagcctggaacagctgctggaagaactggaacaggcgctgcaaaaaatccgcgaactggccgaaaagaaaaactaaggatcc

SEQ ID NO: 4 - protein, 122 aa, Artificial sequence
  Synthetic polypeptide
MHHHHHHENLYFQSEFEKLRQTGDELVQAFQRLREIFDKGDDDSLEQVLEEIEELIQKHRQLFDNRQEAADTEAAKQGDQWVQLFQRFREAIDKGDKDSLEQLLEELEQALQKIRELAEKKN

SEQ ID NO: 5 - protein, 37 aa, Artificial sequence
  Synthetic polypeptide
LGLVAFLIFGLVLILIHLFAAGWVFFAILLLLALILA

SEQ ID NO: 6 - protein, 37 aa, Artificial sequence
  Synthetic polypeptide
LGIILLLAIGLILLAFHLFFAGWLFIAILLFSGIILA

SEQ ID NO: 7 - protein, 109 aa, Artificial sequence
  Synthetic polypeptide
SEFEKLRQTGDEIIQLLQRLREAIDKGDDDSLEQILEELEEAFQKHRQLFENRQEAADTEFAKQGDQWLQLFQRIREAIDKGDKDSLEQLFEESEQGIQKIRELAEKKN

SEQ ID NO: 8 - protein, 101 aa, Artificial sequence
  Synthetic polypeptide
EFEKLRQTGDELVQAFQRLREIFDKGDDDSLEQVLEEIEELIQKHRQLFDNRQEAADTEAAKQGDQWVQLFQRFREAIDKGDKDSLEQLLEELEQALQKIR